(19) United States
(12) Patent Application Publication
(10) Pub. No.: US 2010/0151556 A1
(43) Pub. Date: Jun. 17, 2010

(54) HYBRID AND SINGLE CHAIN MEGANUCLEASES AND USE THEREOF

(75) Inventors: Sylvain Arnould, Saint-Denis (FR); Patrick Chames, Paris (FR); Andre Choulika, Paris (FR); Jean-Charles Epinat, Paris (FR); Sylvestre Grizot, La Garenne Colombes (FR); Emmanuel Lacroix, Bruxelles (BE)

Correspondence Address: OBLON, SPIVAK, MCCLELLAND MAIER & NEUSTADT, L.L.P., 1940 DUKE STREET, ALEXANDRIA, VA, 22314, US

(73) Assignee: CELLECTIS, Romainville (FR)

(21) Appl. No.: 12/482,124

(22) Filed: Jun. 10, 2009

Related U.S. Application Data
(63) Continuation-in-part of application No. PCT/IB2009/000439, filed on Feb. 2, 2009.
Application No. PCT/IB2009/000439 is a continuation-in-part of application No. PCT/IB2008/001331, filed on Jan. 31, 2008.
Application No. PCT/IB2008/001331 is a continuation-in-part of application No. 10/507,736, filed on Jul. 27, 2005.
(60) Provisional application No. 60/364,113, filed on Mar. 15, 2002.

(51) Int. Cl. C12N 7/00; C12N 9/16; C07H 21/04; C12N 15/63

(52) U.S. Cl. 435/235.1; 435/196; 536/23.2; 435/320.1

(57) ABSTRACT

This patent application relates to hybrid and/or single-chain rare-cutting endonucleases, called meganucleases, which recognize and cleave a specific nucleotide sequence, to polynucleotide sequences encoding for said rare-cutting endonucleases, to a vector comprising one of said polynucleotide sequences, to a cell or animal comprising one of said polynucleotide sequences or said rare-cutting endonucleases, to a process for producing one of said rare-cutting endonucleases and any use of the disclosed products and methods. More particularly, this invention contemplates any use of such rare-cutting endonuclease for genetic engineering and gene therapy.

### BACKGROUND

1. Field of the Invention

This patent application relates to hybrid and/or single-chain rare-cutting endonucleases, called meganucleases, which recognize and cleave a specific nucleotide sequence, to polynucleotide sequences encoding for said rare-cutting endonucleases, to a vector comprising one of said polynucleotide sequences, to a cell or animal comprising one of said polynucleotide sequences or said rare-cutting endonucleases, to a process for producing one of said rare-cutting endonucleases and any use of the disclosed products and methods. More particularly, this invention contemplates any use of such rare-cutting endonuclease for genetic engineering and gene therapy.

2. Brief Description of the Prior Art

Meganucleases constitute a family of very rare-cutting endonucleases. It was first characterised at the beginning of the Nineties by the use (in vivo) of the protein I-Sce I (Omega nuclease, originally encoded by a mitochondrial group I intron of the yeast Saccharomyces cerevisiæ). Homing endonucleases encoded by introns ORF, independent genes or intervening sequences (inteins) are defined now as “meganucleases”, with striking structural and functional properties that distinguish them from “classical” restriction enzymes (generally from bacterial system R/MII). They have recognition sequences that span 12-40 by of DNA, whereas “classical” restriction enzymes recognise much shorter stretches of DNA, in the 3-8 by range (up to 12 by for rare-cutter). Therefore, the meganucleases present a very low frequency of cleavage, even in the human genome.

Furthermore, general asymmetry of Meganucleases target sequences contrasts with the characteristic dyad symmetry of most restriction enzyme recognition sites. Several Meganucleases encoded by introns ORF or inteins have been shown to promote the homing of their respective genetic elements into allelic intronless or inteinless sites. By making a site-specific double-strand break in the intronless or inteinless alleles, these nucleases create recombinogenic ends, which engage in a gene conversion process that duplicates the coding sequence and leads to the insertion of an intron or an intervening sequence at the DNA level.

Meganucleases fall into 4 separated families on the basis of pretty well conserved amino acids motifs. One of them is the dodecapeptide family (dodecamer, DOD, D1-D2, LAGLI-DADG, P1-P2). This is the largest family of proteins clustered by their most general conserved sequence motif: one or two copies (vast majority) of a twelve-residue sequence: the di-dodecapeptide. Meganucleases with one dodecapetide (D) are around 20 kDa in molecular mass and act as homodimer. Those with two copies (DD) range from 25 kDa (230 AA) to 50 kDa (HO, 545 AA) with 70 to 150 residues between each motif and act as monomer. Cleavage is inside the recognition site, leaving 4 nt staggered cut with 3′OH overhangs. I-Ceu I, and I-Cre I illustrate the meganucleases with one Dodecapeptide motif (mono-dodecapeptide). I-Dmo I, I-Sce I, PI-Pfu I and PI-Sce I illustrate meganucleases with two Dodecapeptide motifs.

Goguel et al (Mol. Cell. Biol., 1992, 12, 696-705) shows by switching experiments of RNA maturase and meganuclease of yeast mitochondria that the meganuclease badly tolerates sequence switching and loses its endonuclease activity.

Endonucleases are requisite enzymes for today's advanced gene engineering techniques, notably for cloning and analyzing genes. Meganucleases are very interesting as rare-cutter endonucleases because they have a very low recognition and cleavage frequency in large genome due to the size of their recognition site. Therefore, the meganucleases are used for molecular biology and for genetic engineering, more particularly according to the methods described in WO 96/14408, U.S. Pat. No. 5,830,729, WO 00/46385, and WO 00/46386.

Up to now, in a first approach for generating new endonuclease, some chimeric restriction enzymes have been prepared through hybrids between a zinc finger DNA-binding domain and the non-specific DNA-cleavage domain from the natural restriction enzyme Fok I (Smith et al, 2000, Nucleic Acids Res, 28, 3361-9; Kim et al, 1996, Proc Natl Acad Sci USA, 93, 1156-60; Kim & Chandrasegaran, 1994, Proc Natl Acad Sci USA, 91, 883-7; WO 95/09233; WO 94/18313).

An additional approach consisted of an alteration of the recognition domain of EcoRV restriction enzyme in order to change its specificity by site-specific mutagenesis (Wenz et al, 1994, Biochim Biophys Acta, 1219, 73-80).

Despite these efforts, there is still a strong need for new rare-cutting endonucleases with new sequence specificity for the recognition and cleavage.

### SUMMARY

The invention concerns a hybrid meganuclease comprising a first domain and a second domain, said first and second domains being derived from two different initial LAGLIDADG meganucleases, said initial meganucleases being either mono- or di-LAGLIDADG meganucleases. The invention also contemplates a hybrid meganuclease comprising two domains, each domain being derived from the same meganuclease and said two domains having a different arrangement than the initial meganuclease (i.e. the second domain is derived from the N-terminal domain of the initial meganuclease and/or the first domain is derived from the C-terminal of the initial meganuclease).

The invention also concerns a single; chain meganuclease comprising a first domain and a second domain, said first and second domains being derived from an initial mono-LAGLIDADG meganuclease.

The invention further concerns any polynucleotide encoding a hybrid or single-chain meganuclease according to the present invention and vectors, cells or non-human animals comprising such a polynucleotide.

The invention concerns any use of a hybrid or single-chain meganuclease according to the present invention or a polynucleotide encoding it for molecular biology, genetic engineering and gene therapy.

### BRIEF DESCRIPTION OF THE DRAWINGS

FIG. 1 is a ribbon representation of I-DmoI (pdb code 1b24). The discontinuous line represents the two-fold pseudo-symmetry axis between the two domains. The N-terminal domain of I-DmoI is left of that axis, and the C-terminal domain is on the right side. DNA (not present in structure 1b24) should bind perpendicular to the symmetry axis below the two β-sheets (arrows). α^{D} and α′^{D} refer to helices comprising the dodecapeptide motif, DBM to DNA binding moiety, and V to variable sequence.

FIG. 2 is a ribbon representation of dimeric I-CreI with bound DNA in stick representation (pdb code 1g9y). The discontinuous line represents the two-fold symmetry axis between the two domains or monomers. The bound DNA lies perpendicular to the symmetry axis below the two β-sheets (arrows). α^{D} refers to helix comprising the dodecapeptide motif, DBM to DNA binding moiety, and V to variable sequence.

FIGS. 3A and 3B respectively are ribbon representations of I-DmoI and I-CreI in the region of the dodecapeptide motifs. For both proteins, the main chain atoms of residues corresponding to the dodecapeptide motifs are shown in stick representation, together with the superimposed atoms from the other protein. The discontinuous line represents the two-fold symmetry axis between the two protein domains. Stars represent, in FIG. 3A, the limits of the linkers between the I-DmoI domains and in FIG. 3B the corresponding positions in I-CreI where that linker is to be engineered. Orientations of the proteins are as in FIGS. 1 and 2.

FIG. 4 is a ribbon representation of the I-DmoI/I-CreI hybrid protein (model structure built by juxtaposition of the two domains taken from their respective X-ray structures). The linker joining both domains, which is the end of the I-DmoI part, is between the two stars. The discontinuous line represents the two-fold symmetry axis between the two protein domains. The N-term domain of I-DmoI is left and the I-CreI domain right of the axis. α^{D} and α′^{D} refer to helices comprising the dodecapeptide motif, DBM to DNA binding moiety, and V to variable sequence.

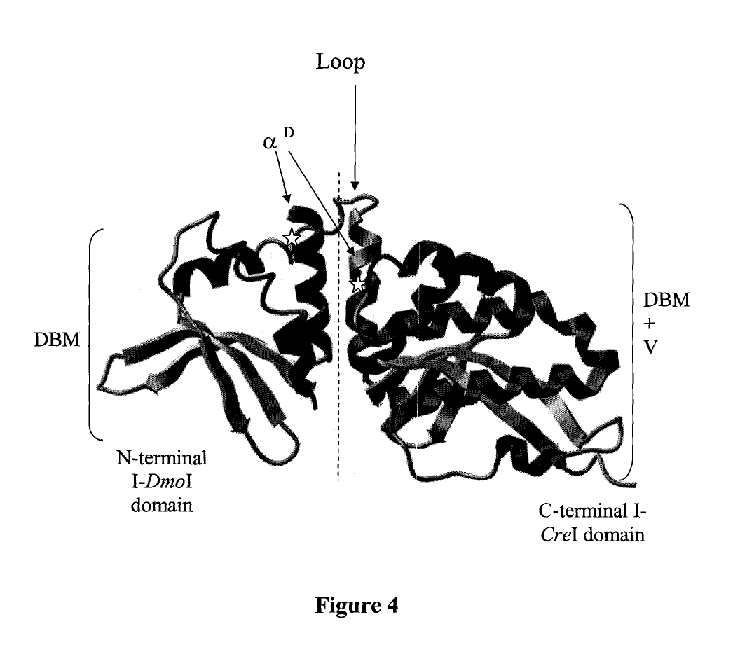

FIGS. 5A and 5B respectively are ribbon representations of the I-CreI dimer (1g9y) and the single chain I-CreI (modelled structure). (FIG. 5A) The stars indicate where the linker is to be introduced; three α-helices in the first monomer (following the left-most star) are removed in the single chain, together with the N-terminal residues of the second monomer (prior to the other star). (FIG. 5B) The loop joining both domains, comprised between the two stars, is taken from I-DmoI (structure 1b24). The grey disk represent the symmetry axis (orientation is from the top of the structures, e.g. compared to the previous figures, it was rotated by 90° around an horizontal axis).

FIGS. 6A and 6B disclose the amino acid sequence of two alternatives of hybrid meganuclease I-Dmo I/I-Cre I and a polynucleotide sequence encoding each alternative. Underlined residues are the LAGLI-DADG motifs. In bold, residues within the I-DmoI domain that are mutated are shown. The “ ” indicates the swap point, at the boundary between the I-DmoI and I-CreI domains. In all protein sequences, the two first N-terminal residues are methionine and alanine (MA), and the three C-terminal residues alanine, alanine and aspartic acid (AAD). These sequences allow having DNA coding sequences comprising the NcoI (CCATGG) and EagI (CGGCCG) restriction sites, which are used for cloning into various vectors. The alternative A just presents a swapping point whereas the alternative B has three additional mutations avoiding potential hindrance.

FIG. 7 discloses the amino acid sequence of an alternative of a single chain I-Cre I and one polynucleotide encoding said single chain meganuclease. In the protein sequence, the two first N-terminal residues are methionine and alanine (MA), and the three C-terminal residues alanine, alanine and aspartic acid (AAD). These sequences allow having DNA coding sequences comprising the NcoI (CCATGG) and EagI (CGGCCG) restriction sites, which are used for cloning into various vectors.

FIG. 8 shows the In vitro cleavage assay for the hybrid meganuclease I-Dmo I/I-Cre I (FIG. 8B) and the SDS-PAGE gel with such hybrid (FIG. 8A). FIG. 8A: Line A: molecular weight markers; Line B: Hybrid meganuclease I-Dmo I/I-Cre I; Line C: Wild type meganuclease I-Dmo I. FIG. 8B: Agarose gel activity of hybrid I-Dmo I/I-Cre; lines A, B, C, D: target I-Dmo I/I-Cre I; lines E, F, G, H: target I-Cre I/I-Dmo I; lines D, G: size markers; lines C, H: linear plasmid; lines B, F: assay at 37° C.; lines A, E: assay at 65° C.

FIG. 9 shows the In vitro cleavage assay for the single chain meganuclease 1-Cre I (FIG. 9B) and the SDS-PAGE gel of the gel filtration with such single chain meganuclease (FIG. 9A). FIG. 9A: Line A: molecular weight markers; Lines B and C: dead colume of gel filtration. Line D: single chain meganuclease I-Cre I. FIG. 9B: Agarose gel activity of single chain meganuclease; Line A: markers; Line B target site of wild type I-Cre I meganuclease; Line C: target site of wild type I-Cre I+wild type I Cre I meganuclease (positive control); Line D: target site of wild type I-Sce I+single chain I-Cre I meganuclease (negative control); Lines E and F: target site of wild type I-Cre I+single chain I-Cre I meganuclease, respectively, at 37 and 65° C. for lines E and F during 1 h.

FIG. 10A is a schematic representation of the LACURAZ reporter construct. LACZ represents the elements of the Lac Z gene. The sections A of each side of the intervening sequence comprise the internal duplication of the Lac Z gene. pADH1 is a yeast constitutive promoter. tADH1 is a yeast terminator. Ura3 represents the Ura3 gene. The arrows represent the transcription beginning. The tag “Cleavage site” refers to the recognition and cleavage site of the assayed meganuclease. “Trp 1” refers to a Trp 1 selectable marker and <<cen>> to an ARS-CEN origin of replication.

FIG. 10B is a schematic representation of the meganuclease inducible expression vector. pGAL10 is a yeast promoter which is inducible in presence of galactose. tADH1 is a yeast terminator. “Leu2” and “2μ” respectively refer to a Leu2 selectable marker and a 2μ origin of replication. The arrow represents the transcription beginning.

FIG. 11 shows the combinations of co-transformations and subsequent modifications of the reporter gene. <<# X>> refers to the combination disclosed in Table A. The light grey boxes refer to Ura3 gene. The dark grey boxes refer to the Lac Z gene. The white boxes refer to the gene encoding the meganuclease. The white tags refer to the recognition and cleavage site of the assayed meganuclease.

FIG. 12 is a Cα ribbon representation of I-CreI.

FIG. 13 is a schematic representation of the human XPC gene (GenBank accession number NC_{—}000003). The XPC exons are boxed. The XPC4.1 (or C1: SEQ ID NO: 42) sequence (position 20438) is situated in Exon 9.

FIG. 14 represents 22 by DNA targets cleaved by I-CreI or some of its derived variants (SEQ ID NO: 37, 43 to 45, 42, 46 and 47 respectively). C1221 is the I-CreI target. 10GAG_P, 10GTA_P and 5TCT_P are palindromic targets, which differ from C1221 by the boxed motifs. C1 is the XPC target; C3 and C4 are palindromic targets, which are derived respectively from the left and the right part of C1. As shown in the Figure, the boxed motifs from 10GAG_P, 10GTA_P and 5TCT_P are found in the C1 target.

FIG. 15 illustrates the C1 target cleavage by heterodimeric combinatorial mutants. The figure displays secondary screening of combinations of C3 and C4 cutters with the C1 target. The H33 mutant is among seven C3 cutters, X2 is among eight C4 cutters and the cleavage of the C1 target by the X2/H33 heterodimer is circled in black.

FIG. 16 represents the pCLS0542 meganuclease expression vector map. pCLS0542 is a 2 micron-based replicative vector marked with a LEU2 auxotrophic gene, and an inducible Gal10 promoter for driving the expression of the meganuclease.

FIG. 17 represents the map of pCLS1088, a plasmid for expression of meganucleases in mammalian cells.

FIG. 18 represents the pCLS1058 reporter vector map. The reporter vector is marked with blasticidine and ampicilline resistance genes. The LacZ tandem repeats share 800 bp of homology, and are separated by 1.3 kb of DNA. They are surrounded by EF1-alpha promoter and terminator sequences. Target sites are cloned using the Gateway protocol (INVITROGEN), resulting in the replacement of the CmR and ccdB genes with the chosen target site

FIG. 19 illustrates the yeast screening of the eighteen single chain constructs against the three XPC targets C1, C3 and C4. Each single chain molecule is referred by its number described in Table 1. For each four dots yeast cluster, the two left dots are the result of the experiment, while the two right dots are various internal controls to assess the experiment quality and validity.

FIG. 20 illustrates the cleavage of the C1, C3 and C4 XPC targets by the two X2-L1-H33 and X2-RM2-H33 single chain constructs in an extrachromosomal assay in CHO cells. Background corresponds to the transfection of the cells with an empty expression vector. Cleavage of the S1234 I-SceI target by I-SceI in the same experiment is shown as a positive control.

FIG. 21 illustrates the cleavage of the C1, C3 and C4 XPC targets by the X2/H33 heterodimer and the two X2-L1-H33_{G19S} and X2-RM2-H33_{G19S} single chain constructs in an extrachromosomal assay in CHO cells. Background corresponds to the transfection of the cells with an empty expression vector. Cleavage of the S1234 I-SceI target by I-SceI in the same experiment is shown as a positive control.

FIG. 22 illustrates the yeast screening of three XPC single chain molecules X2-L1-H33, SCX1 and SCX2 against the three XPC targets (C1, C3 and C4). SCX1 is the X2(K7E)-L1-H33(E8K,G19S) molecule and SCX2 stands for the X2(E8K)-L1-H33(K7E,G19S) molecule. For each four dots yeast cluster, the two left dots are the result of the experiment, while the two right dots are various internal controls to assess the experiment quality and validity.

FIG. 23 is a schematic representation of the Cricetulus griseus Hypoxanthine-Guanine Phosphoribosyl Transferase (HPRT) mRNA (GenBank accession number J00060.1). The ORF is indicated as a grey box. The HprCH3 target site is indicated with its sequence (SEQ ID NO: 50) and position.

FIG. 24 represents 22 by DNA targets cleaved by I-CreI or some of its derived variants (SEQ ID NO: 37, 43 and 48 to 52, respectively). C1221 is the I-CreI target. 10GAG_P, 10CAT_P and 5CTT_P are palindromic targets, which differ from C1221 by the boxed motifs. HprCH3 is the HPRT target, HprCH3.3 and HprCH3.4 are palindromic targets, which are derived respectively from the left and the right part of HprCH3. As shown in the Figure, the boxed motifs from 10 GAG_P, 10CAT_P and 5CTT_P are found in the HprCH3 target.

FIG. 25 illustrates the yeast screening of the MA17 and H33 homodimer, of the HPRT heterodimer and of three HPRT single chain molecules against the three HPRT targets HprCH3, HprCH3.3 and HprCH3.4. H is the MA17/H33 heterodimer. Since it results from co-expression of MA17 and H33, there are actually three molecular species in the yeast: the two MA17 and 1133 homodimers, together with the MA17/H33 heterodimer. Homodimer formation accounts for cleavage of the HprCH3.3 and HprCH3.4 targets. SC1 to SC3 are MA17-L1-H33, MA17-L1-H33_{G19S} and MA17-RM2-H33, respectively. For each four dots yeast cluster, the two left dots are the result of the experiment, while the two right dots are various internal controls to assess the experiment quality and validity.

FIG. 26 illustrates the cleavage of the HprCH3, HprCH3.3 and HprCH3.4 HPRT targets by the MA17/H33 heterodimer and the four HPRT single chain constructs (MA17-L1-H33, MA17-L1-H33_{G19S}, MA17-RM2-H33 and MA17-RM2-H33_{G19S}) in an extrachromosomal assay in CHO cells. Background corresponds to the transfection of the cells with an empty expression vector. Cleavage of the S1234 I-SceI target by I-SceI in the same experiment is shown as a positive control.

FIG. 27 is a schematic representation of the human RAG1 gene (GenBank accession number NC_{—}000011). Exonic sequences are boxed, and the Exon-Intron junctions are indicated. ORF is indicated as a grey box. The RAG1.10 sequence is indicated with its sequence (SEQ ID NO: 57) and position.

FIG. 28 represents 22 by DNA targets cleaved by I-CreI or some of its derived variants (SEQ ID NO: 37 and 53 to 59, respectively). C1221 is the I-CreI target. 10GTT_P, 5CAG_P, 10TGG_P and 5GAG_P are palindromic targets, which differ from C1221 by the boxed motifs. RAG1.10 is the RAG1 target, RAG1.10.2 and RAG1.10.3 are palindromic targets, which are derived from the left and the right part of RAG1.10, respectively. As shown in the Figure, the boxed motifs from 10GTT_P, 5CAG_P, 10TGG_P and 5GAG_P are found in the RAG1.10 target.

FIG. 29 illustrates the yeast screening of the four RAG1 single chain molecules against the three RAG1 targets RAG1.10, RAG1.10.2 and RAG1.10.3. SC1 to SC4 represent M2-L1-M3, M2_{G19S}-L1-H33, M2-RM2-H33 and M2_{G19S}-RM2-M3, respectively. Activity of the M2 and M3I-CreI mutants and the M2/M3 heterodimer against the three RAG1 targets is also shown. For each four dots yeast cluster, the two left dots are the result of the experiment, while the two right dots are various internal controls to assess the experiment quality and validity.

FIG. 30 illustrates the yeast screening of two single chain molecules SC1 and SC2 against the three RAG1.10 targets. SC1 is the M3-RM2-M2 molecule and SC2 stands for the M3(K7E K96E)-RM2-M2(E8K E61R) molecule. For each 4 dots yeast cluster, the two left dots are the result of the experiment, while the two right dots are various internal controls to assess the experiment quality and validity.

FIG. 31 illustrates the yeast screen of six RAG1 single chain molecules against the three RAG1 targets RAG1.10, RAG1.10.2 and RAG1.10.3. Activity of the M2/M3 heterodimer against the three RAG1 targets is also shown. For each 4 dots yeast cluster, the two left dots are the result of the experiment, while the two right dots are various internal controls to assess the experiment quality and validity.

FIG. 32 illustrates the cleavage of the RAG1.10, RAG1.10.2 and RAG1.10.3 targets by four single chain constructs in an extrachromosomal assay in CHO cells. Background corresponds to the transfection of the cells with an empty expression vector. Results are expressed in percentage of the activity of the M2/M3 heterodimer against the same three targets.

FIG. 33 illustrates: A. Principle of the chromosomal assay in CHO cells. B. Gene correction activity of the M2/M3 heterodimer and the two single chain molecules M3-RM2-M2_{G19S} and M3⁻-RM2-M2⁺_{G19S}. The frequency of LacZ positive cells is represented in function of the amount of transfected expression plasmid.

FIG. 34 illustrates the activity of five single chain molecules against the three RAG1.10, RAG1.10.2 and RAG1.10.3 targets. In each yeast cluster, the two left dots are a single chain molecule, while the two right dots are experiment internal controls.

FIG. 35 is a diagram of the gene targeting strategy used at the endogenous RAG1 locus. The RAG1 target sequence (RAG1.10: SEQ ID NO: 57) is located just upstream of exon 2 coding for the Rag1 protein. Exon 2 is boxed, with the open reading frame in white. Cleavage of the native RAG1 gene by the meganuclease yields a substrate for homologous recombination, which may use the repair matrix containing 1.7 kb of exogenous DNA flanked by homology arms as a repair matrix.

FIG. 36 represents pCLS1969 vector map.

FIG. 37 illustrates the PCR analysis of gene targeting events. Clones wild type for the RAG1 locus and clones having a random insertion of the donor repair plasmid will not result in PCR amplification. Clones having a gene targeting event at the RAG1 locus result in a 2588 bp PCR product.

FIG. 38 illustrates the Southern blot analysis of cell clones. Genomic DNA preparations were digested with HindIII and Southern blotting was performed with a fragment of the RAG1 gene lying outside the right homology arm. The locus maps indicate the restriction pattern of the wild-type locus (5.3 kb) and the targeted locus (3.4 kb). The probe is indicated by a solid black box. Five clones (1-5) samples derived from single transfected cells are analyzed, together with DNA from non transfected cells (293). In three samples, one of the alleles has been targeted. H, HindIII site.

FIG. 39 illustrates the biophysical analysis of the heterodimers and the single chain molecule. A; Circular dichroism spectrum of the single-chain meganuclease and the corresponding heterodimeric protein. B; Thermal denaturation of all the heterodimers and the single-chain variant. C; Calibration graph of partition coefficient (KAV) versus the logarithm of the molecular mass of four protein standards (open circles) for an analytical Superdex-200 10-300GL column. The value of the single-chain meganuclease is indicated with a filled circle (measured MW=34 kDa, theoretical MW=41 kDa)

FIG. 40 illustrates toxicity study. A. Dose response study. CHO cell lines were transfected with various amounts of expression vector for various meganucleases and a fixed quantity of the repair plasmid. ♦, M2/M3 heterodimer; ▪, M2_{G19S}/M3 heterodimer; ▴, M2⁺/M3⁻ heterodimer; X, M2⁺_{G19S}/M3⁻; *, M3-RM2-M2_{G19S}; , M2⁺_{G19S}-RM2-M3⁻; □, I-SceI. B. Toxicity of the engineered meganucleases, as monitored by a cell survival assay. Various amounts of meganuclease expression vector and a constant amount of plasmid encoding GFP were used to cotransfect CHO-K1 cells. Cell survival is expressed as the percentage of cells expressing GFP six days after transfection, as described in the Materials and Methods. The totally inactive M2_{G19S}/M3_{G19S} heterodimer is shown as a control for non toxicity (⋄). C. DNA damage was also visualized by the formation of γH2AX foci at DNA double-strand breaks. Representative images of cells treated with 10 times the active dose of meganuclease.

### DETAILED DESCRIPTION

### Definitions

In the present application, by “meganuclease” is intended a rare-cutting endonuclease, typically having a polynucleotide recognition site of about 12-40 bp in length, more preferably of 14-40 bp. Typical meganucleases cause cleavage inside their recognition site, leaving 4 nt staggered cut with 3′OH overhangs. The meganuclease are also commonly called homing endonuclease. Preferably, “meganucleases” according to the present invention belong to the dodecapeptide family (LAGLIDADG homing endonuclease family). For more information on meganucleases, see Dalgaard et al (1997, Nucleic Acids Research, 25, 4626-4638) and Chevalier and Stoddard (2001, Nucleic Acids Research, 29, 3757-3774). Specific examples of LAGLIDADG meganucleases are listed in Table 4.

By “helix” or “helices” is intended in the present invention α-helix or α-helices. α^{D} and α^{LAGLIDADG} in the present invention refers to the helix comprising the LAGLIDADG, DOD or dodecapeptide motif.

By “derived” is intended that the domain comprises the sequence of a domain of the meganuclease from which the domain is derived. Said sequence of a domain can comprise some modifications or substitutions.

By “meganuclease domain” or “domain” is intended the region which interacts with one half of the DNA target of a meganuclease and is able to associate with the other domain of the same meganuclease which interacts with the other half of the DNA target to form a functional meganuclease able to cleave said DNA target.

By “single-chain meganuclease” is intended a meganuclease comprising two LAGLIDADG homing endonuclease domains linked by a peptidic spacer. The single-chain meganuclease is able to cleave a chimeric DNA target sequence comprising one different half of the two parent meganucleases target sequences. The single-chain meganuclease is also named single-chain derivative, single-chain meganuclease, single-chain meganuclease derivative or chimeric meganuclease.

By “core domain” is intended the “LAGLIDADG homing endonuclease core domain” which is the characteristic α₁β₁β₂α₂β₃β₄α₃ fold of the homing endonucleases of the LAGLIDADG family, corresponding to a sequence of about one hundred amino acid residues. Said core domain comprises four beta-strands (β₁β₂β₃β₄) folded in an antiparallel beta-sheet which interacts with one half of the DNA target. This core domain is able to associate with another LAGLIDADG homing endonuclease core domain which interacts with the other half of the DNA target to form a functional endonuclease able to cleave said DNA target. For example, in the case of the dimeric homing endonuclease I-CreI (163 amino acids), the core domain comprises the residues 6 to 94 of I-CreI.

By “beta-hairpin” is intended two consecutive beta-strands of the antiparallel beta-sheet of a LAGLIDADG homing endonuclease core domain (β₁β₂ or β₃β₄) which are connected by a loop or a turn.

By “subdomain” is intended the region of a LAGLIDADG homing endonuclease core domain which interacts with a distinct part of a homing endonuclease DNA target half-site.

By “meganuclease variant” or “variant” is intended a meganuclease obtained by replacement of at least one residue in the amino acid sequence of the wild-type meganuclease (natural meganuclease) with a different amino acid.

By “functional variant” is intended a variant which is able to cleave a DNA target sequence, preferably said target is a new target which is not cleaved by the parent meganuclease. For example, such variants have amino acid variation at positions contacting the DNA target sequence or interacting directly or indirectly with said DNA target.

By “meganuclease variant with novel specificity” is intended a variant having a pattern of cleaved targets different from that of the parent meganuclease. The terms “novel specificity”, “modified specificity”, “novel cleavage specificity”, “novel substrate specificity” which are equivalent and used indifferently, refer to the specificity of the variant towards the nucleotides of the DNA target sequence.

By “I-CreI” is intended the wild-type I-CreI (Protein Data Bank accession number 1g9y), corresponding to the sequence SEQ ID NO: 36 in the sequence listing.

By “parent I-CreI monomer” or “I-CreI monomer” is intended the full-length wild-type I-CreI amino acid sequence SEQ ID NO: 36 (163 amino acids) or a functional variant thereof comprising amino acid substitutions in SEQ ID NO: 36. I-CreI functions as a dimer, which is made of two I-CreI monomers.

By “portion of said parent I-CreI monomer which extends at least from the beginning of the first alpha helix to the end of the C-terminal loop of I-CreI and includes successively: the α₁β₁β₂α₂β₃β₄α₃ core domain, the α₄ and α₅ helices and the C-terminal loop” is intended the amino acid sequence corresponding to at least positions 8 to 143 of I-CreI.

A “target site” or recognition and/or cleavage site, DNA target”, “DNA target sequence”, “target sequence”, “target-site”, “target”, “site”; “site of interest”, “recognition sequence”, “homing recognition site”, “homing site” as used herein, refers to a polynucleotide sequence bound and cleaved by a meganuclease. These terms refer to a distinct DNA location, preferably a genomic location, at which a double stranded break (cleavage) is to be induced by the meganuclease. Preferably, said DNA target is a 20 to 24 by double-stranded palindromic, partially palindromic (pseudo-palindromic) or non-palindromic polynucleotide sequence that is recognized and cleaved by a LAGLIDADG homing endonuclease, or a variant, or a single-chain chimeric meganuclease. The DNA target is defined by the 5′ to 3′ sequence of one strand of the double-stranded polynucleotide. Cleavage of the DNA target usually occurs at the nucleotide positions +2 and −2, respectively for the sense and the antisense strand. Unless otherwise indicated, the position at which cleavage of the DNA target by a meganuclease occurs, corresponds to the cleavage site on the sense strand of the DNA target.

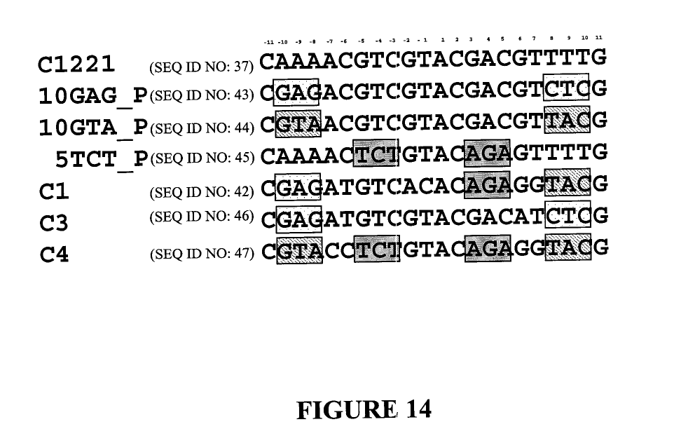

By “I-CreI site” is intended a 22 to 24 by double-stranded DNA sequence which is cleaved by I-CreI. I-CreI sites include the wild-type (natural) non-palindromic I-CreI homing site and the derived palindromic sequences such as the sequence 5′-c₋₁₁a₋₁₀a₋₉a₋₈a₋₇c₋₆g₋₅t₋₄c₋₃g⁻²t₋₁a₊₁c₊₂g₊₃a₊₄c₊₅g₊₆t₊₇t₊₈t₊₉t₊₁₀g₊₁₁ also called C1221 (SEQ ID NO:37; FIG. 14).

By “DNA target half-site”, “half cleavage site” or half-site” is intended the portion of the DNA target which is bound by each LAGLIDADG homing endonuclease core domain.

By “chimeric DNA target” or “hybrid DNA target” is intended the fusion of a different half of two parent meganuclease target sequences. In addition at least one half of said target may comprise the combination of nucleotides which are bound by at least two separate subdomains (combined DNA target).

By “chimeric DNA target comprising one different half of each parent homodimeric I-CreI meganuclease target sequence” is intended the target sequence comprising the left part of the palindromic target sequence cleaved by the homodimeric meganuclease made of two identical monomers of one parent monomer and the right part of the palindromic target sequence cleaved by the homodimeric meganuclease made of two identical monomers of the other parent monomer.

The term “recombinant polypeptide” is used herein to refer to polypeptides that have been artificially designed and which comprise at least two polypeptide sequences that are not found as contiguous polypeptide sequences in their initial natural environment, or to polypeptides which have been expressed from a recombinant polynucleotide.

As used herein, the term “individual” includes mammals, as well as other animals (e.g., birds, fish, reptiles, insects). The terms “mammal” and “mammalian”, as used herein, refer to any vertebrate animal, including monotremes, marsupials and placental, that suckle their young and either give birth to living young (eutharian or placental mammals) or are egg-laying (metatharian or nonplacental mammals).

Examples of mammalian species include humans and other primates (e.g., monkeys, chimpanzees), rodents (e.g., rats, mice, guinea pigs) and ruminents (e.g., cows, pigs, horses).

The term “reporter gene”, as used herein, refers to a nucleic acid sequence whose product can be easily assayed, for example, colorimetrically as an enzymatic reaction product, such as the lacZ gene which encodes for -galactosidase. Examples of widely-used reporter molecules include enzymes such as β-galactosidase, β-glucoronidase, β-glucosidase; luminescent molecules such as green flourescent protein and firefly luciferase; and auxotrophic markers such as His3p and Ura3p. (See, e.g., Chapter 9 in Ausubel, F. M., et al. Current Protocols in Molecular Biology, John Wiley & Sons, Inc. (1998)).

Amino acid residues in a polypeptide sequence are designated herein according to the one-letter code, in which, for example, Q means Gln or Glutamine residue, R means Arg or Arginine residue and D means Asp or Aspartic acid residue.

Nucleotides are designated as follows: one-letter code is used for designating the base of a nucleoside: a is adenine, t is thymine, c is cytosine, and g is guanine. For the degenerated nucleotides, r represents g or a (purine nucleotides), k represents g or t, s represents g or c, w represents a or t, m represents a or c, y represents t or c (pyrimidine nucleotides), d represents g, a or t, v represents g, a or c, b represents g, t or c, h represents a, t or c, and n represents g, a, t or c.

As used interchangeably herein, the terms “nucleic acid” “oligonucleotide”, and “polynucleotide” include RNA, DNA, or RNA/DNA hybrid sequences of more than one nucleotide in either single chain or duplex form. The term “polynucleotide” refers to a polymer of units comprising a purine or pyrimidine, a ribose or deoxyribose sugar moiety, and a phosphate group, or phosphodiester linkage. “polynucleotides” also refers to polynucleotide comprising “modified nucleotides” which comprise at least one of the following modifications (a) an alternative linking group, (b) an analogous form of purine, (c) an analogous form of pyrimidine, or (d) an analogous sugar.

Endonuclease: By “endonuclease” is intended an enzyme capable of causing a double-stranded break in a DNA molecule at highly specific locations.

“Cells,” or “host cells”, are terms used interchangeably herein. It is understood that such terms refer not only to the particular subject cell but to the progeny or potential progeny of such a cell. Because certain modifications may occur in succeeding generations due to either mutation or environmental influences, such progeny may not be identical to the parent cell, but are still included within the scope of the term as used herein. As used herein, a cell refers to a prokaryotic cell, such as a bacterial cell, or eukaryotic cell, such as an animal, plant or yeast cell.

“Identity” refers to sequence identity between two nucleic acid molecules or polypeptides. Identity can be determined by comparing a position in each sequence which may be aligned for purposes of comparison. When a position in the compared sequence is occupied by the same base, then the molecules are identical at that position. A degree of similarity or identity between nucleic acid or amino acid sequences is a function of the number of identical or matching nucleotides at positions shared by the nucleic acid sequences. Various alignment algorithms and/or programs may be used to calculate the identity between two sequences, including FAST A, or BLAST which are available as a part of the GCG sequence analysis package (University of Wisconsin, Madison, Wis.), and can be used with, e.g., default settings.

By “homologous” is intended a sequence with enough identity to another one to lead to a homologous recombination between sequences, more particularly having at least 95% identity, preferably 97% identity and more preferably 99%.

By “mutation” is intended the substitution, deletion, insertion of one or more nucleotides/amino acids in a polynucleotide (cDNA, gene) or a polypeptide sequence. Said mutation can affect the coding sequence of a gene or its regulatory sequence. It may also affect the structure of the genomic sequence or the structure/stability of the encoded mRNA.

By “site-specific mutation” is intended the mutation of a specific nucleotide/codon in a nucleotidic sequence as opposed to random mutation.

The “non-human animals” of the invention include mammalians such as rodents, non-human primates, sheep, dog, cow, chickens, amphibians, reptiles, etc. Preferred non-human animals are selected from the rodent family including rat and mouse, most preferably mouse, though transgenic amphibians, such as members of the Xenopus genus, and transgenic chickens, cow, sheep can also provide important tools.

The term “vector” refers to a nucleic acid molecule capable of transporting another nucleic acid to which it has been linked. One type of preferred vector is an episome, i.e., a nucleic acid capable of extra-chromosomal replication. Preferred vectors are those capable of autonomous replication and/or expression of nucleic acids to which they are linked. Vectors capable of directing the expression of genes to which they are operatively linked are referred to herein as “expression vectors”. A vector according to the present invention comprises, but is not limited to, a YAC (yeast artificial chromosome), a BAC (bacterial artificial), a baculovirus vector, a phage, a phagemid, a cosmid, a viral vector, a plasmid, a RNA vector or a linear or circular DNA or RNA molecule which may consist of a chromosomal, non chromosomal, semi-synthetic or synthetic DNA. In general, expression vectors of utility in recombinant DNA techniques are often in the form of “plasmids” which refer generally to circular double stranded DNA loops which, in their vector form are not bound to the chromosome. Large numbers of suitable vectors are known to those of skill in the art and commercially available, such as the following bacterial vectors: pQE70, pQE60. pQE-9 (Qiagen), pbs, pD1O, phagescript, psiX174. pbluescript SK. pbsks. pNH8A. pNH16A, pNH18A, pNH46A (Stratagene); ptrc99a, pKK223-3, pKK233-3, pDR540, pRIT5 (Pharmacia); pWLNEO. pSV2CAT, pOG44, pXT1, pSG (Stratagene); pSVK3, pBPV, pMSG, pSVL (Pharmacia); pQE-30 (QIAexpress).

Viral vectors include retrovirus, adenovirus, parvovirus (e.g., adenoassociated viruses), coronavirus, negative strand RNA viruses such as orthomyxovirus (e.g., influenza virus), rhabdovirus (e.g., rabies and vesicular stomatitis virus), paramyxovirus (e.g. measles and Sendai), positive strand RNA viruses such as picornavirus and alphavirus, and double stranded DNA viruses including adenovirus, herpesvirus (e.g., Herpes Simplex virus types 1 and 2, Epstein-Barr virus, cytomegalovirus), and poxvirus (e.g., vaccinia, fowlpox and canarypox). Other viruses include Norwalk virus, togavirus, flavivirus, reoviruses, papovavirus, hepadnavirus, and hepatitis virus, for example. Examples of retroviruses include: avian leukosis-sarcoma, mammalian C-type, B-type viruses, Dtype viruses, HTLV-BLV group, lentivirus, spumavirus (Coffin, J. M., Retroviridae: The viruses and their replication, In Fundamental Virology, Third Edition, B. N. Fields, et al., Eds., Lippincott-Raven Publishers, Philadelphia, 1996). Other examples include murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency virus, simian sarcoma virus, Rous sarcoma virus and lentiviruses. Other examples of vectors are described, for example, in McVey et al., U.S. Pat. No. 5,801,030, the teachings of which are incorporated herein by reference.

Vectors can comprise selectable markers (for example, neomycin phosphotransferase, histidinol dehydrogenase, dihydrofolate reductase, hygromycin phosphotransferase, herpes simplex virus thymidine kinase, adenosine deaminase, glutamine synthetase, and hypoxanthine-guanine phosphoribosyl transferase for eukaryotic cell culture; TRP1 for S. cerevisiae; tetracycline, rifampicin or ampicillin resistance in E. coli; etc. . . . ). However, the invention is intended to include such other forms of expression vectors which serve equivalent functions and which become known in the art subsequently hereto.

Flanked: A polynucleotide to be linearized or excised is flanked by a cleavage site if such a site is present at or near either or both ends of the polynucleotide. There can be one cleavage site present or near one end of the polynucleotide to be linearized or excised or there can be two cleavage sites, one at or near each end of the polynucleotide to be linearized or excised. By “near” is preferably intended in the present invention that the cleavage site is located at less than 1 kb, preferably less than 500 bp, more preferably less than 200, or 100 bp, of the end of the polynucleotide to be integrated.

The present invention relates to new designed rare-cutting endonucleases and use thereof. These new designed rare-cutting endonucleases are preferably derived from the meganuclease family of “dodecapeptide” LAGLIDADG.

### Hybrid Meganucleases

Meganucleases form a class of over 200 rare-cutting double-stranded DNA endonucleases (group I intron homing endonucleases and inteins) (Belfort and Roberts, 1997, Nucleic Acids Res, 25, 3379-3388; Jurica and Stoddard, 1999, Cell Mol Life Sci, 55, 1304-1326). They recognize asymmetrical DNA sequences that are between 14 and 40 base pairs in length, producing double-strand breaks at about the center of their target sequence. Said target site is defined herein as the sum of two different half-sites. In complex DNA, 16 (and over) nucleotides-long DNA sequences can be expected to be unique, even in a genome the size of the human genome (3×10⁹ base pairs). Meganucleases will thus cut cellular genomes only once, at the target locus.

The LAGLIDADG protein family is characterized by the presence of one or two copies of a well-conserved sequence motif, termed dodecapeptide, or P1 and P2, LAGLI and DADG or LAGLIDADG. Outside of these motifs, there is no relevant sequence homology (overall pairwise sequence homologies are below 25%). The smaller examples, i.e. I-CreI (Durrenberger and Rochaix, 1991, Embo J, 10, 3495-3501), have only one dodecapeptide motif and function as homodimers of two 15-20 kDa subunits or domains. These proteins having only one dodecapeptide motif are called in the present application as “mono-dodecapeptide” proteins. Larger proteins, i.e. I-DmoI (Dalgaard et al., 1993, Proc Natl Acad Sci USA, 90, 5414-5417), I-SceI (Jacquier et Dujon, 1985, Cell, 41, 383-394) and PI-SceI (Gimble and Wang, 1996, J Mol Biol, 263, 163-180), on the other hand, are single-chain proteins (20-30 kDa) bearing two (non-identical) dodecapeptide motifs. These proteins having two dodecapeptide motifs are called in the present application as “di-dodecapeptide” proteins.

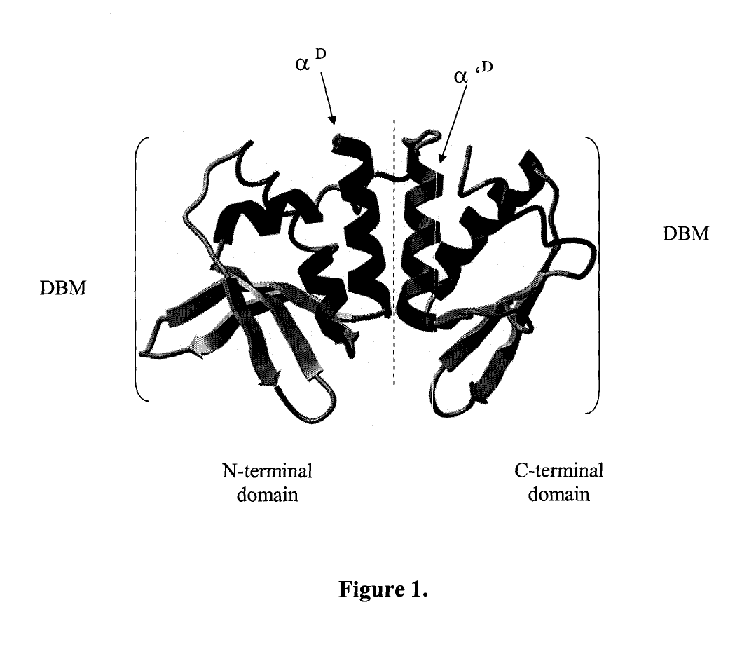

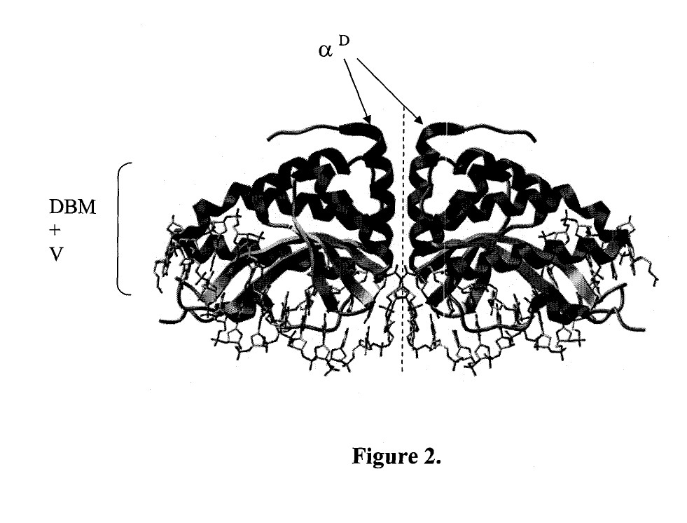

Detailed three-dimensional structures (Chevalier et al., 2001, Nat Struct Biol, 8, 312-316; Duan et al., 1997, Cell, 89, 555-564; Heath et al., 1997, Nat Struct Biol, 4, 468-476; Hu et al., 2000, J Biol Chem, 275, 2705-2712; Ichiyanagi et al., 2000, J Mol Biol, 300, 889-901; Jurica et al., 1998, Mol Cell, 2, 469-476; Poland et al., 2000, J Biol Chem, 275, 16408-16413; Silva et al., 1999, J Mol Biol, 286, 1123-1136), have been solved for four LAGLIDADG proteins: I-CreI (FIG. 1), I-DmoI (FIG. 2), PI-SceI and PI-PfuI. These structures illustrate that the dodecapeptide motifs are part of a two-helix bundle. The two α-helices form most of the central interface, where a two-fold (pseudo-) symmetry axis separates two structural domains. In addition to the dodecapeptide motif, each domain presents a DNA binding interface that drives the protein towards interacting with one of the two half sites of the target DNA sequence.

A unique catalytic, active site comprises amino acid residues from both structural domains, whose specific nature and spatial distribution is required for DNA cleavage. In the LAGLIDADG protein family, the residues in the active sites are divergent. The only residues that display persistent conservation are the last acidic amino acids (D or E) from both LAGLIDADG motifs (underlined residue). Therefore, it is difficult to assign functional roles to residues in the active site, except for those acidic amino acids. Mutations of those residues abolish catalysis, but not DNA binding (Lykke-Andersen et al., 1997, Embo J, 16, 3272-3281; Gimble & Stephens, 1995, J. Biol. Chem., 270, 5849-5856). Besides, a hydration shell, consisting of several water molecules structurally organized by the amino acid side chains of acidic and basic residues, together with divalent cations, has probably an essential role in conducting the cleavage of DNA phosphodiester bonds (Chevalier et al., 2001, Nat Struct Biol, 8, 312-316).

Engineering known meganucleases, in order to modify their specificity towards DNA sequences could allow targeting of new DNA sequences, and to produce double-strand breaks in chosen genes.

However, residues related to the inter-domain packing interface and the catalytic site are very constrained. It is known that the catalytic domains of enzymes are often complex and highly reactive to modifications. In the case of the meganucleases, this sensibility to modification is increased, as the catalytic site is constituted by the interface of two domains. Consequently, it is not known whether domain swapping of meganucleases, which have distinct catalytic site residues, would restore functional, active proteins. Moreover, despite the domain structure of known meganucleases, particularly those of the LAGLIDADG protein family, nothing is known about the modular behaviour of such domain structure.

For the first time, the present invention shows that LAGLIDADG endonucleases are modular and that domain swapping of natural homing endonucleases or meganucleases is both possible and fruitful: novel, artificial combinations of two domains taken from different LAGLIDADG meganucleases recognize, bind and cut DNA sequences made of the corresponding two half-sites. Engineering such artificial combinations (hybrid or chimerical homing endonucleases or meganucleases) is primarily useful in order to generate meganucleases with new specificity.

The LAGLIDADG protein family essentially shows a sequence conservation in the dodecapeptide motifs. The 3D structure are similar: they have the same set of secondary structure elements organized with a unique topology. Conservation of the dodecapeptide motif and protein size (in particular, the separation distance in sequence length between two dodecapeptide motifs in di-dodecameganuclease together with the biological relationships, i.e. same “function” and conserved 3D architecture) are thought sufficient to propose that the secondary structure is conserved.

The present invention concerns the novel endonucleases, more particularly hybrid meganucleases, preferably derived from at least two different LAGLIDADG meganucleases. The initial meganucleases can be “mono-dodecapeptide” or “mono-LAGLIDADG”, such as I-Cre I meganuclease, or “di-dodecapeptide” or “di-LAGLIDADG” meganucleases such as I-Dmo I. These new designed endonucleases or meganucleases are hybrid of LAGLIDADG meganucleases. The invention concerns a hybrid meganuclease comprising two domains, each domain being derived from a different LAGLIDADG meganuclease. See Table 4 (Motif “D” refers to mono-dodecapeptide meganucleases and motif “dd” or “DD” to di-dodecapeptide meganucleases). The invention also contemplates a hybrid meganuclease comprising two domains, each domain being derived from the same meganuclease but in a different arrangement (e.g., location, organization, position, etc.) as compared to the initial meganuclease (e.g., the second domain is derived from the N-terminal domain of the initial meganuclease and/or the first domain is derived from the C-terminal of the initial meganuclease).

By “domain” of LAGLIDADG meganucleases is intended in the present invention a polypeptide fragment comprising or consisting of a dodecapeptide motif and a DNA binding moiety. Optionally, the domain can also comprise additional polypeptide sequences not involved in the DNA binding nor in the domain interface. However, those additional sequences have variable size and are generally not critical for the DNA recognition and binding nor the endonuclease activity. The dodecapeptide motif is involved in an α-helix, herein schematically called α^{LAGLIDADG} or α^{D}. In more detail, the last D(E) residue is generally capping the α-helix and the following Gly residue initiates a main chain re-direction into a β-strand perpendicular to the α-helix. The DNA binding moiety, herein schematically called DBM, generally comprises α-helices and β-strands. The minimal DNA binding moiety in a meganuclease is a β-hairpin (2 β-strands connected by a loop or turn). Natural meganucleases comprise two such β-hairpins in each DNA binding moiety, connecting into a 4-stranded β-sheet. The connecting between the two β-hairpins comprises an α-helix. The DNA binding moiety generally comprises a further α-helix downstream of the 4-stranded β-sheet. The additional polypeptide sequences could be found at each side of the group consisting of the dodecapeptide motif and the DNA binding moiety. Therefore, a meganuclease domain according to the present invention comprises the helix comprising the dodecapeptide motif, α^{D}, and a DNA binding moiety, DBM. Optionally, an additional sequence can be further comprised in said domain. Said additional sequence is possible at the N-terminal side of a first domain of the hybrid meganuclease or at the C-terminal side of a second domain of the hybrid meganuclease.

The LAGLIDADG meganucleases comprising two dodecapeptide motifs, herein called di-LAGLIDADG meganuclease, comprise two domains, one domain called N-terminal domain and the other C-terminal domain. The N-terminal domain consecutively comprises an additional optional sequence, the dodecapeptide motif and the DNA binding moiety. The C-terminal domain consecutively comprises the dodecapeptide motif, the DNA binding moiety and an additional optional sequence. The two dodecapeptide α-helices of each domain form a tightly packed domain interface. The loop connecting the two domains is between the DNA binding moiety of the N-terminal domain and the helix comprising the second dodecapeptide motif of the C-terminal domain. The di-dodecapeptide meganucleases could schematically be represented by the following structure from the N-terminal end to the C-terminal end: V α^{D} DBM (L) α′^{D} DBM′ V′ (V referring to additional optional sequence, α^{D} to the helix comprising the dodecapeptide motif, DBM to the DNA binding moiety, L to the connecting loop; the refers to the elements of the C-terminal domain). The helices α^{D} and α′^{D} correspond to the helices comprising the dodecapeptide motifs. The domains of a meganuclease comprising two dodecapeptide motifs are asymmetric (similar but generally not identical). Number of di-dodecapeptide meganucleases are known. See Table 4, Motif “dd” or “DD”. (Also see, Dalgaard et al., 1993, Proc Natl Acad Sci USA, 90, 5414-5417, Table 4 and FIG. 1)

The dimeric LAGLIDADG meganucleases comprising one dodecapeptide motif, herein called mono-dodecapeptide meganucleases, consecutively comprises an additional optional polypeptide sequence, the dodecapeptide motif, the DNA binding moiety, and an additional optional polypeptide sequence. The two dodecapeptide helices (one in each monomer) form a tightly packed dimer interface. The mono-dodecapeptide meganucleases could schematically be represented by the following structure comprising from the N-terminal end to the C-terminal end: V α^{D} DBM V′ (V and V′ referring to additional optional sequences, α^{D} to the helix comprising the dodecapeptide motif, DBM to the DNA binding moiety). Number of mono-dodecapeptide meganucleases are known. See Table 4, Motif D (Also see Lucas et al., 2001, Nucleic Acids Res., 29, 960-9, Table 4 and FIG. 1).

Therefore, the invention concerns a hybrid meganuclease comprising or consisting of a first domain and a second domain in the orientation N-terminal toward C-terminal, said first and second domains being derived from two different initial LAGLIDADG meganucleases, said initial meganucleases being either mono- or di-dodecapeptide meganucleases and said first and second domains being bound by a convenient linker and wherein said hybrid meganuclease is capable of causing DNA cleavage. The invention also contemplates a hybrid meganuclease comprising or consisting of two domains, each domain being derived from the same meganuclease, said two domains having a different arrangement than the initial meganuclease (i.e. the second domain is derived from the N-terminal domain of the initial meganuclease and/or the first domain is derived from the C-terminal of the initial meganuclease) and said first and second domains being bound by a convenient linker.

The initial mono- and di-dodecapeptide meganucleases according to the present invention for the generation of hybrid meganucleases are preferably selected from the group consisting of the meganucleases listed in the Table 4, notably I-Sce I, I-Chu I, I-Dmo I, I-Cre I, I-Csm I, PI-Sce I, PI-Tli I, PI-Mtu I, I-Ceu I, I-Sce II, I-Sce III, HO, PI-Civ I, PI-Ctr I, PI-Aae I, PI-Bsu I, PI-Dha I, PI-Dra I, PI-Mav I, PI-Mch I, PI-Mfu I, PI-Mfl I, PI-Mga I, PI-Mgo I, PI-Min I, PI-Mka I, PI-Mle I, PI-Mma I, PI-Msh I, PI-Msm I, PI-Mth I, PI-Mtu I, PI-Mxe I, PI-Npu I, PI-Pfu I, PI-Rma I, PI-Spb I, PI-Ssp I, PI-Fac I, PI-Mja I, PI-Pho I, PI-Tag I, PI-Thy I, PI-Tko I, and PI-Tsp I; preferably, I-Sce I, I-Chu I, I-Dmo I, I-Cre I, I-Csm I, PI-Pfu I, PI-Sce I, PI-Tli I, PI-Mtu I, I-Ceu I, I-Sce II, I-Sce III, and HO; more preferably, I-Sce I, I-Chu I, I-Dmo I, I-Cre I, I-Csm I, PI-Sce I, PI-Pfu I, PI-Tli I, PI-Mtu I, and I-Ceu I; still more preferably I-Dmo I, I-Cre I, I-Sce I, and I-Chu I; or, even more preferably I-Dmo I, and I-Cre I.

The initial di-dodecapeptide meganucleases according to the present invention for the generation of hybrid meganuclease are preferably selected from the group consisting of the meganucleases comprising a “DD” or “dd” motif listed in the Table 4, notably: I-Sce I, I-Chu I, I-Dmo I, I-Csm I, PI-Sce I, PI-Tli I, PI-Mtu I, I-Sce II, I-Sce III, HO, PI-Civ I, PI-Ctr I, PI-Aae I, PI-Bsu I, PI-Dha I, PI-Dra I, PI-Mav I, PI-Mch I, PI-Mfu I, PI-Mfl I, PI-Mga I, PI-Mgo I, PI-Min I, PI-Mka I, PI-Mle I, PI-Mma I, PI-Msh I, PI-Msm I, PI-Mth I, PI-Mtu I, PI-Mxe I, PI-Npu I, PI-Pfu I, PI-Rma I, PI-Spb I, PI-Ssp I, PI-Fac I, PI-Mja I, PI-Pho I, PI-Tag I, PI-Thy I, PI-Tko I, and PI-Tsp I; preferably, I-Sce I, I-Chu I, I-Dmo I, I-Csm I, PI-Pfu I, PI-Sce I, PI-Tli I, PI-Mtu I, I-Sce II, I-Sce III, and HO; more preferably, I-Sce I, I-Chu I, I-Dmo I, I-Csm I, PI-Sce I, PI-Tli I, and PI-Mtu I; still more preferably I-Dmo I, I-Sce I, and I-Chu I; or even more preferably I-Dmo I.

The initial mono-dodecapeptide meganucleases according to the present invention for the generation of hybrid meganucleases are preferably selected from the group consisting of the meganucleases comprising a “D” motif listed in the Table 4, notably: I-Cre I, I-Ceu I; preferably, I-Cre I.

More particularly, the present invention concerns the hybrid meganuclease comprising or consisting of a first domain from a mono- or di-dodecapeptide meganuclease and a second domain from another mono- or di-dodecapeptide meganuclease, said first and second domains being bound by a convenient linker. In a preferred embodiment, the invention concerns a hybrid meganuclease selected from the group consisting of the following hybrid meganucleases:

I-Sce I/I-Chu I, I-Sce I/PI-Pfu I, I-Chu I/I-Sce I, I-Chu I/PI-Pfu I, I-Sce I/I-Dmo I, I-Dmo I/I-Sce I, I-Dmo I/PI-Pfu I, I-Dmo I/I-Cre I, I-Cre I/I-Dmo I, I-Cre I/PI-Pfu I, I-Sce I/I-Csm I, I-Sce I/I-Cre I, I-Sce I/PI-Sce I, I-Sce I/PI-Tli I, I-Sce I/PI-Mtu I, I-Sce I/I-Ceu I, I-Cre I/I-Ceu I, I-Chu I/I-Cre I, I-Chu I/I-Dmo I, I-Chu I/I-Csm I, I-Chu I/PI-Sce I, I-Chu I/PI-Tli I, I-Chu I/PI-Mtu I, I-Cre I/I-Chu I, I-Cre I/I-Csm I, I-Cre I/PI-Sce I, I-Cre I/PI-Tli I, I-Cre I/PI-Mtu I, I-Cre I/I-SceI, I-Dmo I/I-Chu I, I-Dmo I/I-Csm I, I-Dmo I/PI-Sce I, I-Dmo I/PI-Tli I, I-Dmo I/PI-Mtu I, I-Csm I/I-Chu I, I-Csm I/PI-Pfu I, I-Csm I/I-Cre I, I-Csm I/I-Dmo I, I-Csm I/PI-Sce I, I-Csm I/PI-Tli I, I-Csm I/PI-Mtu I, I-Csm I/I-Sce I, PI-Sce I/I-Chu I, PI-Sce I/I-Pfu I, PI-Sce I/I-Cre I, PI-Sce I/I-Dmo I, PI-Sce I/I-Csm I, PI-Sce I/PI-Tli I, PI-Sce I/PI-Mtu I, PI-Sce I/I-Sce I, PI-Tli I/I-Chu I, PI-Tli I/PI-Pfu I, PI-Tli I/I-Cre I, PI-Tli I/I-Dmo I, PI-Tli I/I-Csm I, PI-Tli I/PI-Sce I, PI-Tli I/PI-Mtu I, PI-Tli I/I-Sce I, PI-Mtu I/I-Chu I, PI-Mtu I/PI-Pfu I, PI-Mtu I/I-Cre I, PI-Mtu I/I-Dmo I, PI-Mtu I/I-Csm I, PI-Mtu I/PI-Sce I, PI-Mtu I/PI-Tli I, and PI-Mtu I/I-SceI;

Preferably, I-Sce I/I-Chu I, I-Sce I/PI-Pfu I, I-Chu I/I-Sce I, I-Chu I/PI-Pfu I, I-Sce I/I-Dmo I, I-Dmo I/I-Sce I, I-Dmo I/PI-Pfu I, I-Dmo I/I-Cre I, I-Cre I/I-Dmo I, I-Cre I/PI-Pfu I, I-Sce I/I-Csm I, I-Sce I/I-Cre I, I-Sce I/PI-Sce I, I-Sce I/PI-Tli I, I-Sce I/PI-Mtu I, I-Sce I/I-Ceu I, I-Chu I/I-Cre I, I-Chu/I-Dmo I, I-Chu I/I-Csm I, I-Chu I/PI-Sce I, I-Chu I/PI-Tli I, I-Chu I/PI-Mtu I, I-Cre I/I-Chu I, I-Cre I/I-Csm I, I-Cre I/PI-Sce I, I-Cre I/PI-Tli I, I-Cre I/PI-Mtu I, I-Cre, I/I-SceI, I-Dmo I/I-Chu I, I-Dmo I/I-Csm I, I-Dmo I/PI-Sce I, I-Dmo I/PI-Tli I, I-Dmo I/PI-Mtu I, I-Csm I/I-Chu I, I-Csm I/PI-Pfu I, I-Csm I/I-Cre I, I-Csm I/I-Dmo I, I-Csm I/PI-Sce I, I-Csm I/PI-Tli I, I-Csm I/PI-Mtu I, I-Csm I/I-Sce I, PI-Sce I/I-Chu I, PI-Sce I/I-Pfu I, PI-Sce I/I-Cre I, PI-Sce I/I-Dmo I, PI-Sce I/I-Csm I, PI-Sce I/PI-Tli I, PI-Sce I/PI-Mtu I, PI-Sce I/I-Sce I, PI-Tli I/I-Chu I, PI-Tli I/PI-Pfu I, PI-Tli I/I-Cre I, PI-Tli I/I-Dmo I, PI-Tli I/I-Csm I, PI-Tli I/PI-Sce I, PI-Tli I/PI-Mtu I, PI-Tli I/I-Sce I, PI-Mtu I/I-Chu I, PI-Mtu I/PI-Pfu I, PI-Mtu I/I-Cre I, PI-Mtu I/I-Dmo I, PI-Mtu I/I-Csm I, PI-Mtu I/PI-Sce I, PI-Mtu I/PI-Tli I, and PI-Mtu I/I-SceI;

More preferably I-Sce I/I-Chu I, I-Chu I/I-Sce I, I-Sce I/I-Dmo I, I-Dmo I/I-Sce I, I-Dmo I/I-Cre I, and I-Cre I/I-Dmo I;

Still more preferably I-Dmo I/I-Cre I, and I-Cre I/I-Dmo I; or,

Even more preferably I-Dmo I/I-Cre I; more particularly the hybrid meganuclease of SEQ ID No 2 or 4.

For example, for “I-Sce I/I-Ceu I”, the first indicated meganuclease corresponds to the origin of the first domain of the hybrid meganuclease and the second indicated meganuclease to the origin of the second domain of the hybrid meganuclease.

Optionally, said hybrid meganuclease comprises or consists of:

1) a first domain from a di-dodecapeptide meganuclease and a second domain from another di-dodecapeptide meganuclease, said first and second domains being bound by a convenient linker;

2) a first domain from a mono-dodecapeptide meganuclease and a second domain from another di-dodecapeptide meganuclease, said first and second domains being bound by a convenient linker;

3) a first domain from a di-dodecapeptide meganuclease and a second domain from another mono-dodecapeptide meganuclease, said first and second domains being bound by a convenient linker; or

4) a first domain from a mono-dodecapeptide meganuclease and a second domain from the same or another mono-dodecapeptide meganuclease, preferably from another mono-dodecapeptide meganuclease, said first and second domains being bound by a convenient linker.

Preferably, said hybrid meganuclease comprises or consists of:

1) a first domain from a di-dodecapeptide meganuclease and a second domain from another di-dodecapeptide meganuclease, said first and second domains being bound by a convenient linker; or,

2) a first domain from a di-dodecapeptide meganuclease and a second domain from another mono-dodecapeptide meganuclease, said first and second domains being bound by a convenient linker.

More preferably, said hybrid meganucleases comprise or consists of a first domain from a di-dodecapeptide meganuclease and a second domain from another di-dodecapeptide meganuclease, said first and second domains being bound by a convenient linker.

Optionally, said hybrid meganuclease comprises or consists of:

1) a first domain derived from the N-terminal domain of a di-dodecapeptide meganuclease and a second domain derived from the C-terminal domain of another di-dodecapeptide meganuclease, said first and second domains being bound by a convenient linker;

2) a first domain derived from the N-terminal domain of a di-dodecapeptide meganuclease and a second domain derived from the N-terminal domain of the same or another di-dodecapeptide meganuclease, said first and second domains being bound by a convenient linker;

3) a first domain derived from the N-terminal domain of a di-dodecapeptide meganuclease and a second domain derived from another mono-dodecapeptide meganuclease, said first and second domains being bound by a convenient linker;

4) a first domain derived from the C-terminal domain of a di-dodecapeptide meganuclease and a second domain derived from the C-terminal domain of the same or another di-dodecapeptide meganuclease, said first and second domains being bound by a convenient linker;

5) a first domain derived from a mono-dodecapeptide meganuclease and a second domain derived from the C-terminal domain of another di-dodecapeptide meganuclease, said first and second domains being bound by a convenient linker;

6) a first domain derived from the C-terminal domain of a di-dodecapeptide meganuclease and a second domain derived from the N-terminal domain of the same or another di-dodecapeptide meganuclease, said first and second domains being bound by a convenient linker;

7) a first domain derived from a mono-dodecapeptide meganuclease and a second domain derived from the N-terminal domain of another di-dodecapeptide meganuclease, said first and second domains being bound by a convenient linker;

8) a first domain derived from the C-terminal domain of a di-dodecapeptide meganuclease and a second domain derived from another mono-dodecapeptide meganuclease, said first and second domains being bound by a convenient linker; or,

9) a first domain derived from a mono-dodecapeptide meganuclease and a second domain derived from the same or another mono-dodecapeptide meganuclease, said first and second domains being bound by a convenient linker.

Preferably, said hybrid meganuclease comprises or consists of:

1) a first domain derived from the N-terminal domain of a di-dodecapeptide meganuclease and a second domain derived from the C-terminal domain of another di-dodecapeptide meganuclease, said first and second domains being bound by a convenient linker;

2) a first domain derived from the N-terminal domain of a di-dodecapeptide meganuclease and a second domain derived from the N-terminal domain of the same or another di-dodecapeptide meganuclease, said first and second domains being bound by a convenient linker;

3) a first domain derived from the N-terminal domain of a di-dodecapeptide meganuclease and a second domain derived from another mono-dodecapeptide meganuclease;

4) a first domain derived from the C-terminal domain of a di-dodecapeptide meganuclease and a second domain derived from the C-terminal domain of the same or another di-dodecapeptide meganuclease, said first and second domains being bound by a convenient linker; or,

5) a first domain derived from a mono-dodecapeptide meganuclease and a second domain derived from the C-terminal domain of another di-dodecapeptide meganuclease, said first and second domains being bound by a convenient linker.

More preferably, said hybrid meganuclease comprises or consists of:

1) a first domain derived from the N-terminal domain of a di-dodecapeptide meganuclease and a second domain derived from the C-terminal domain of another di-dodecapeptide meganuclease, said first and second domains being bound by a convenient linker;

2) a first domain derived from the N-terminal domain of a di-dodecapeptide meganuclease and a second domain derived from the N-terminal domain of the same or another di-dodecapeptide meganuclease, said first and second domains being bound by a convenient linker; or,

3) a first domain derived from the N-terminal domain of a di-dodecapeptide meganuclease and a second domain derived from another mono-dodecapeptide meganuclease, said first and second domains being bound by a convenient linker.

Are also contemplated in the present invention the hybrid meganucleases comprising or consisting of a first domain and a second domain from the same di-dodecapeptide meganuclease, said first and second domains being bound by a convenient linker, if the ordering of the domain is not the same of the initial meganuclease. More particularly, are contemplated in the present invention the hybrid meganuclease comprising a first and a second domains in the orientation N-terminal toward C-terminal, wherein each domain are derived from the same di-dodecapeptide meganuclease and said first domain is derived from the C-terminal domain and said second domain is derived from the N-terminal domain.

The means for introducing a link between the two domains of the hybrid meganuclease is well known by one man skilled in the art. In the present invention, the preferred means are either the use of a flexible polypeptide linker or the use of a loop from a di-dodecapeptide meganuclease. In the present invention, the loop is an embodiment of the linker. The flexible polypeptide linker essentially comprises glycine, serine and threonine residues. The loop can be either a loop present in one of the 2 initial di-dodecapeptide meganucleases used for design the hybrid meganuclease or a loop from any other di-dodecapeptide meganuclease, preferably the I-Dmo I loop, which is introduced between the two domains.

Our preferred approach for generating hybrid meganuclease is a domain swapping consistent with the various LAGLIDADG meganucleases.

N-Terminal Domain of Di-Dodecapeptide Meganuclease/C-Terminal Domain of Di-Dodecapeptide Meganuclease

A first preferred embodiment concerns a hybrid meganuclease comprising or consisting of a first domain derived from the N-terminal domain of a di-dodecapeptide meganuclease (A) and a second domain derived from the C-terminal domain of another di-dodecapeptide meganuclease (B), said first and second domains being bound by a convenient linker. The swapping point is positioned at any convenient place. The swapping point is the point at which the sequence of the first meganuclease (A) is substantially replaced by the sequence of the second meganuclease (B). This swapping point can be positioned from the last helix of the DNA binding moiety DBM to the end of the helix comprising the second dodecapeptide motif, α′^{D}. It is preferably positioned within the loop (L) preceding the second dodecapeptide motif (α′^{D}) or in the helix (α′^{D}) comprising the dodecapeptide motif. Generally, few amino acids, about 4 to 10 amino acids, upstream the dodecapeptide motif, also participate to the formation of the helix (α′^{D}). In one preferred embodiment, the swapping point is positioned within the helix (α′^{D}). In a particularly preferred embodiment, the swapping point is positioned in the helix (α′^{D}) before the dodecapeptide motif itself. The resulting hybrid meganuclease comprises the N-terminal domain of the meganuclease A and the C-terminal domain of the meganuclease B. Such hybrid meganuclease schematically comprises:

| | V | | | | | | V′ |
|---|---|---|---|---|---|---|---|
| Type | optional | α^{D} | DBM | L | α′^{D} | DBM′ | optional |
| 1 | A | αA | A(N) | A | α′A | B(C) | B |
| 2 | A | αA | A(N) | A | α′A/α′B | B(C) | B |
| 3 | A | αA | A(N) | A | α′B | B(C) | B |
| 4 | A | αA | A(N) | A/B | α′B | B(C) | B |
| 5 | A | αA | A(N) | B | α′B | B(C) | B |
| 6 | A | αA | A(N)/B(N) | B | α′B | B(C) | B |

A and B indicating the meganuclease at the origin of the segment V, α^{D}, DBM, L, α′^{D}, DBM′, V′. A/B indicates that the swapping point is into the segment, the origin of the first part of the element is A and that of the second part is B. αA and αB refer to the α^{D} of the N-terminal domain and α′A and α′B refer to the α′^{D} of the C-terminal domain. For “DBM” column, the letter (N) and (C), respectively, indicate the origin from the N-terminal domain and the C-terminal domain.

In an alternative embodiment, the swapping point is replaced by a “swapping domain”. Indeed, instead of abruptly changing the sequence, the helices α^{D} and α′^{D} of the hybrid meganuclease can be a mixture of the two initial meganucleases. The amino acid residues from the helices α^{D} and α′^{D} which are directed towards the helices interface are those of one meganuclease for both helices α^{D} and α′^{D} (either those of α^{D} and α′^{D} from the meganuclease A or those of α^{D} and α′^{D} from the meganuclease B). Optionally, the residues at the interface could be derived from another pair of dodecapeptide helices from a mono- or di-dodecapeptide meganuclease X. Within each domain, the amino acid residues from the helix comprising α^{D} and α′^{D} which are directed towards the inside of the domain are those corresponding to the residues found at that position in that domain of the meganuclease it comes from (those of α^{D} from the meganuclease A and those of α′^{D} from the meganuclease B). Such hybrid meganuclease schematically comprises:

| | | α^{D} | | | | α′^{D} | | | V′ |
|---|---|---|---|---|---|---|---|---|---|
| Type | V optional | intra | inter | DBM | L | inter | intra | DBM′ | optional |
| 1 | A | αA | αA | A(N) | A | α′A | α′B | B(C) | B |
| 2 | A | αA | αB | A(N) | A | α′B | α′B | B(C) | B |
| 3 | A | αA | αX | A(N) | A | α′X | α′B | B(C) | B |
| 4 | A | αA | αX | A(N) | A | αX | α′B | B(C) | B |
| 5 | A | αA | αA | A(N) | A/B | α′A | α′B | B(C) | B |
| 6 | A | αA | αB | A(N) | A/B | α′B | α′B | B(C) | B |
| 7 | A | αA | αX | A(N) | A/B | α′X | α′B | B(C) | B |
| 8 | A | αA | αX | A(N) | A/B | αX | α′B | B(C) | B |
| 9 | A | αA | αA | A(N) | B | α′A | α′B | B(C) | B |
| 10 | A | αA | αB | A(N) | B | α′B | α′B | B(C) | B |
| 11 | A | αA | αX | A(N) | B | α′X | α′B | B(C) | B |
| 12 | A | αA | αX | A(N) | B | αX | α′B | B(C) | B |
| 13 | A | αA | αA | A(N)/B(N) | B | α′A | α′B | B(C) | B |
| 14 | A | αA | αB | A(N)/B(N) | B | α′B | α′B | B(C) | B |
| 15 | A | αA | αX | A(N)/B(N) | B | α′X | α′B | B(C) | B |
| 16 | A | αA | αX | A(N)/B(N) | B | αX | α′B | B(C) | B |

A, B, and X indicating the meganuclease at the origin of the segment V, α^{D}, DBM, L, α′^{D}, DBM′, V′. A/B indicates that the swapping point is into the segment, the origin of the first part of the element is A and that of the second part is B. “inter” refers to the residues of α^{D} and α′^{D} towards the interface between the domains, and “intra” refers to the residues of α^{D} and α′^{D} towards the inside of each domain. αA and αB refer to the α^{D} of the N-terminal domain and α′A and α′B refer to the α′^{D} of the C-terminal domain. For “DBM” column, the letter (N) and (C), respectively, indicate the origin from the N-terminal domain and the C-terminal domain.

Optionally, some amino acid modifications can be further introduced in order to avoid the steric hindrance between amino acid side chains and/or to increase the stability. Optionally, some amino acid modifications can be further introduced in order to enhance the production and/or the solubility and to decrease the toxicity (Turmel et al, 1997, Nucleic Acid Research, 25, 2610-2619). Optionally, the loop can be completely or partially replaced by a convenient linker. A convenient linker is preferably flexible. Said flexible linker preferably comprises glycine, serine and threonine residues. Short flexible linker can also be introduced between the loop and the domains. Optionally, the loop can also be replaced by a loop from any other di-dodecapeptide meganuclease, preferably the I-Dmo I loop.

Optionally, such hybrid meganuclease comprising or consisting of a first domain from the N-terminal domain of a di-dodecapeptide meganuclease (A) and a second domain from the C-terminal domain of another di-dodecapeptide meganuclease (B), said first and second domains being bound by a convenient linker, can further comprise, at its N and/or C-terminal end, a loop or linker and any additional domain. Preferably, said additional domain is a DNA binding domain, a transcription activator or repressor domain, a nuclear localization signal, or a DNA cleavage domain. Optionally, the endonuclease activity of such hybrid can be abolished. Thus, the main function of hybrid is a specific DNA binding.

N-Terminal Domain of Di-Dodecapeptide Meganuclease/N-Terminal Domain of Di-Dodecapeptide Meganuclease

A second embodiment concerns a hybrid meganuclease comprising or consisting of a first domain derived from the N-terminal domain of a di-dodecapeptide meganuclease (A) and a second domain derived from the N-terminal domain of another di-dodecapeptide meganuclease (B), said first and second domains being bound by a convenient linker. The swapping point is positioned at any convenient place. It is preferably positioned at the end of the loop (LA) preceding the second dodecapeptide motif (α′^{D}) or in the helix (α′^{D}). In one preferred embodiment, the swapping point is positioned within the helix (α′^{D}). In a particularly preferred embodiment, the swapping point is positioned in the helix (α′^{D}) before the dodecapeptide motif itself. The resulting hybrid meganuclease comprises, from the N-terminal end to C-terminal end, the N-terminal domain of the meganuclease A and the N-terminal domain of the meganuclease B. Preferably, the resulting hybrid meganuclease does not comprise the DBM of the C-terminal domain of the meganuclease B, more preferably its C-terminal domain. Such hybrid meganuclease schematically comprises:

| Type | V optional | α^{D} | DBM | L | α′D | DBM′ | V′ optional |
|---|---|---|---|---|---|---|---|
| 1 | A | αA | A(N) | A | α′A | B(N) | B |
| 2 | A | αA | A(N) | A | α′A/αB | B(N) | B |
| 3 | A | αA | A(N) | A | αB | B(N) | B |

A and B indicating the meganuclease at the origin of the segment V, α^{D}, DBM, L, α′^{D}, DBM′, V′. A/B indicates that the swapping point is into the segment, the origin of the first part of the element is A and that of the second part is B. αA and αB refer to the α^{D} of the N-terminal domain and α′A and α′B refer to the α′^{D} of the C-terminal domain. For “DBM” column, the letter (N) and (C), respectively, indicate the origin from the N-terminal domain and the C-terminal domain.

In an alternative embodiment, the swapping point is replaced by a swapping domain. Indeed, instead of abruptly change the sequence, the helices α^{D} and α′^{D} of the hybrid meganuclease can be a mixture of the two initial meganucleases. The amino acid residues from the helices α^{D} and α′^{D} which are directed towards the helix interface are those of one meganuclease for both helices α^{D} and α′^{D} (those of α^{D} and α′^{D} from either the meganuclease A or B). Optionally, the residues at the interface could be derived from another pair of dodecapeptide helices from a mono- or di-dodecapeptide meganuclease X. Within each domain, the amino acid residues from the helices α^{D} and α′^{D} which are directed towards the inside of the domain are those corresponding to the residues found at that position in that domain of the meganuclease it comes from (those of α^{D} from the meganuclease A and those of α^{D} from the meganuclease B). Such hybrid meganuclease schematically comprises:

| | V op- | α^{D} | | | | α′^{D} | | | V′, |
|---|---|---|---|---|---|---|---|---|---|
| Type | tional | intra | inter | DBM | L | inter | intra | DBM′ | optional |
| 1 | A | αA | αA | A(N) | A | α′A | αB | B(N) | B |
| 2 | A | αA | αB | A(N) | A | α′B | αB | B(N) | B |
| 3 | A | αA | αX | A(N) | A | α′X | αB | B(N) | B |
| 4 | A | αA | αX | A(N) | A | αX | αB | B(N) | B |

A, B, and X indicating the meganuclease at the origin of the segment V, α^{D}, DBM, L, α′^{D}, DBM′, V′. “inter” refers to the residues of α^{D} and α′^{D} towards the interface between the domains, and “intra” refers to the residues of α^{D} and α′^{D} towards the inside of each domain. αA and αB refer to the α^{D} of the N-terminal domain and α′A and α′B refer to the α′^{D} of the C-terminal domain. For “DBM” column, the letter (N) and (C), respectively, indicate the origin from the N-terminal domain and the C-terminal domain.

Optionally, some amino acid modifications can be further introduced in order to avoid the steric hindrance between amino acid side chains and/or to increase the stability. Optionally, some amino acid modifications can be further introduced in order to enhance the production and/or the solubility and to decrease the toxicity (Turmel et al, 1997, Nucleic Acod Research, 25, 2610-2619). Optionally, the Loop can be completely or partially replaced by a convenient linker. A convenient linker is preferably flexible. Said flexible linker essentially comprises glycine, serine and threonine residues. Short flexible linker can also be introduced between the loop and the domains. Optionally, the loop can also be replaced by a loop from any other di-dodecapeptide meganuclease, preferably the I-Dmo I loop.

Optionally, such hybrid meganuclease comprising a first domain from the N-terminal domain of a di-dodecapeptide meganuclease (A) and a second domain from the N-terminal domain of another di-dodecapeptide meganuclease (B) can further comprise, at its N and/or C-terminal end, a loop or linker and any additional domain. Preferably, said additional domain is a DNA binding domain, a transcription activator or repressor domain, a nuclear localization signal, or a DNA cleavage domain. Optionally, the endonuclease activity of such hybrid can be abolished.

A hybrid meganuclease comprising or consisting of a first domain derived from the N-terminal domain of a di-dodecapeptide meganuclease (A) and a second domain derived from the N-terminal domain of the same di-dodecapeptide meganuclease (A), said first and second domains being bound by a convenient linker, is also contemplated in the present invention. The same rules of design are applied to this kind of meganuclease.

N-Terminal Domain of Di-Dodecapeptide Meganuclease/Domain of Mono-Dodecapeptide Meganuclease

A third embodiment concerns a hybrid meganuclease comprising or consisting of a first domain from the N-terminal domain of a di-dodecapeptide meganuclease (A) and a second domain from another mono-dodecapeptide meganuclease (B), said first and second domains being bound by a convenient linker. The swapping point is positioned at any convenient place. It is preferably positioned at the end of the loop (LA) preceding the second dodecapeptide motif (α′^{D}) of the meganuclease (A) or in the helix α^{D}. In one preferred embodiment, the swapping point is positioned within the helix α′^{D}. In a particularly preferred embodiment, the swapping point is positioned in the helix α′^{D} before the dodecapeptide motif itself. The resulting hybrid meganuclease comprises, from the N-terminal end to C-terminal end, the N-terminal domain of the meganuclease A and the domain of the meganuclease B. Such hybrid meganuclease schematically comprises:

| Type | V optional | α^{D} | DBM | L | α′^{D} | DBM′ | V′ optional |
|---|---|---|---|---|---|---|---|
| 1 | A | αA | A(N) | A | α′A | B | B |
| 2 | A | αA | A(N) | A | α′A/αB | B | B |
| 3 | A | αA | A(N) | A | αB | B | B |

A and B indicating the meganuclease at the origin of the segment V, α^{D}, DBM, L, α′^{D}, DBM′, V′. A/B indicates that the swapping point is into the segment, the origin of the first part of the element is A and that of the second part is B. αA and αB refer to the α^{D} of the N-terminal domain and α′A and α′B refer to the α′^{D} of the C-terminal domain. For “DBM” column, the letter (N) and (C), respectively, indicate the origin from the N-terminal domain and the C-terminal domain.

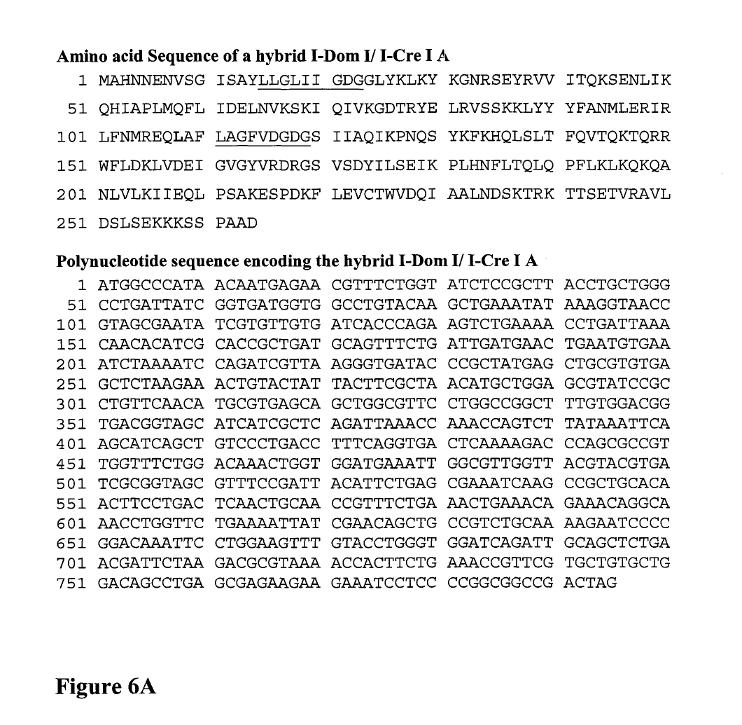

The invention concerns more particularly a hybrid meganuclease I-Dmo I/I-Cre I comprising or consisting of a first domain from the N-terminal domain of I-Dmo I meganuclease and a second domain from I-Cre I meganuclease, said first and second domains being bound by a convenient linker. Preferably, said convenient linker is the I-Dmo I meganuclease loop. Preferably, the swapping point is positioned in the helix α′^{D} before the dodecapeptide motif itself. In one embodiment, the invention concerns the hybrid meganucleases I-Dmo I/I-Cre I disclosed in example 1 and in FIG. 6 or a variant thereof.

In an alternative embodiment, the swapping point is replaced by a swapping domain. Indeed, instead of abruptly change the sequence, the helices α^{D} and α′^{D} of the hybrid meganuclease can be a mixture of the two initial meganucleases. The amino acid residues from the helices α^{D} and α′^{D} which are directed towards the helix interface are those of one meganuclease for both helices (those of α^{D} and α′^{D} from the meganuclease A, or those of α^{D} from the meganuclease B). Optionally, the residues at the interface could be derived from another pair of dodecapeptide helices from a mono- or di-dodecapeptide meganuclease X. Within each domain, the amino acid residues from the helices α^{D} and α′^{D} which are directed towards the inside of the domain are those corresponding to the residues found at that position in that domain of the meganuclease it comes from (those of α^{D} from the meganuclease A and those of α^{D} from the meganuclease B). Such hybrid meganuclease schematically comprises:

| | V op- | α^{D} | | | | α′^{D} | | | V′ |
|---|---|---|---|---|---|---|---|---|---|
| Type | tional | intra | inter | DBM | L | inter | intra | DBM′ | optional |
| 1 | A | αA | αA | A(N) | A | α′A | αB | B | B |
| 2 | A | αA | αB | A(N) | A | αB | αB | B | B |
| 3 | A | αA | αX | A(N) | A | α′X | αB | B | B |
| 4 | A | αA | αX | A(N) | A | αX | αB | B | B |

A, B, and X indicating the meganuclease at the origin of the segment V, α^{D}, DBM, L, α′^{D}, DBM′, V′. A/B indicates that the swapping point is into the segment, the origin of the first part of the element is A and that of the second part is B. “inter” refers to the residues of α^{D} and α′^{D} towards the interface between the domains, and “intra” refers to the residues of α^{D} and α′^{D} towards the inside of each domain. αA and αB refer to the α^{D} of the N-terminal domain and α′A and α′B refer to the α′^{D} of the C-terminal domain. For “DBM” column, the letter (N) and (C), respectively, indicate the origin from the N-terminal domain and the C-terminal domain.

Optionally, some amino acid modifications can be further introduced in order to avoid the steric hindrance between amino acid side chains and/or to increase the stability. Optionally, some amino acid modifications can be further introduced in order to enhance the production and/or the solubility and to decrease the toxicity (Turmel et al, 1997, Nucleic Acod Research, 25, 2610-2619). Optionally, the loop can be completely or partially replaced by a convenient linker. A convenient linker is preferably flexible. Said flexible linker preferably comprises glycine, serine and threonine residues. Short flexible linker can also be introduced between the loop and the domains. Optionally, the loop can also be replaced by a loop from any other di-dodecapeptide meganuclease, preferably the I-Dmo I loop.

Optionally, such hybrid meganuclease comprising a first domain from the N-terminal domain of a di-dodecapeptide meganuclease (A) and a second domain from the domain of another mono-dodecapeptide meganuclease (B) can further comprise, at its N-terminal and/or C-terminal end, a loop or linker and any additional domain. Preferably, said additional domain is a DNA binding domain, a transcription activator or repressor domain, a nuclear localization signal or a DNA cleavage domain. Optionally, the endonuclease activity of such hybrid can be abolished.

C-Terminal Domain of Di-Dodecapeptide Meganuclease/C-Terminal Domain of Di-Dodecapeptide Meganuclease

A forth preferred embodiment concerns a hybrid meganuclease comprising or consisting of a first domain derived from the C-terminal domain of a di-dodecapeptide meganuclease (A) and a second domain derived from the C-terminal domain of another di-dodecapeptide meganuclease (B), said first and second domains being bound by a convenient linker. The swapping point is positioned at any convenient place. This swapping point can be positioned from the last helix of the DNA binding moiety DBM to the beginning of the loop (LB) preceding the helix α′^{D}. It is preferably positioned at the beginning of the loop (LB) preceding the helix α′^{D}. The resulting hybrid meganuclease comprises the C-terminal domain of the meganucleases A and B. Preferably, the resulting hybrid meganuclease does not comprise the DBM of the N-terminal domain of the meganuclease A, more preferably its N-terminal domain. Such hybrid meganuclease schematically comprises:

| | V, | | | | | | |
|---|---|---|---|---|---|---|---|
| Type | optional | α^{D} | DBM | L | α′^{D} | DBM′ | V′ optional |
| 1 | A | α′A | A(C) | B | α′B | B(C) | B |
| 2 | A | α′A | A(C)/B(N) | B | α′B | B(C) | B |

A and B indicating the meganuclease at the origin of the segment V, α^{D}, DBM, L, α′^{D}, DBM′, V′. A/B indicates that the swapping point is into the segment, the origin of the first part of the element is A and that of the second part is B. αA and αB refer to the α^{D} of the N-terminal domain and α′A and α′B refer to the α′^{D} of the C-terminal domain. For “DBM” column, the letter (N) and (C), respectively, indicate the origin from the N-terminal domain and the C-terminal domain.

In an alternative embodiment, the swapping point is replaced by a “swapping domain”. Indeed, instead of abruptly change the sequence, the helices α^{D} and α′^{D} of the hybrid meganuclease can be a mixture of the two initial meganucleases. The amino acid residues from the helices α^{D} and α′^{D} which are directed towards the helices interface are those of one meganuclease for both helices α^{D} and α′^{D} (those of α^{D} and α′^{D} from the meganuclease A or those of α^{D} and α′^{D} from the meganuclease B). Optionally, the residues at the interface could be derived from another pair of dodecapeptide helices from a mono- or di-dodecapeptide meganuclease X. Within each domain, the amino acid residues from the helices α^{D} and α′^{D} which are directed towards the inside of the domain are those corresponding to the residues found at that position in that domain of the meganuclease it comes from (those of α′^{D} from the meganuclease A and those of α′^{D} from the meganuclease B). Such hybrid meganuclease schematically comprises:

| | | α^{D} | | | | α′^{D} | | | |
|---|---|---|---|---|---|---|---|---|---|
| Type | V optional | intra | inter | DBM′ | L | inter | intra | DBM′ | V′ optional |
| 1 | A | α′A | αA | A(C) | B | α′A | α′B | B(C) | B |
| 2 | A | α′A | αB | A(C) | B | α′B | α′B | B(C) | B |
| 3 | A | α′A | αX | A(C) | B | α′X | α′B | B(C) | B |
| 4 | A | α′A | αX | A(C) | B | αX | α′B | B(C) | B |
| 5 | A | α′A | αA | A(C)/B(N) | B | α′A | α′B | B(C) | B |
| 6 | A | α′A | αB | A(C)/B(N) | B | α′B | α′B | B(C) | B |
| 7 | A | α′A | αX | A(C)/B(N) | B | α′X | α′B | B(C) | B |
| 8 | A | α′A | αX | A(C)/B(N) | B | αX | α′B | B(C) | B |

A, B, and X indicating the meganuclease at the origin of the segment V, α^{D}, DBM, L, α′^{D}, DBM′, V′. A/B indicates that the swapping point is into the segment, the origin of the first part of the element is A and that of the second part is B. “inter” refers to the residues of α^{D} and α′^{D} towards the interface between the domains, and “intra” refers to the residues of α^{D} and α′^{D} towards the inside of each domain. αA and αB refer to the α^{D} of the N-terminal domain and α′A and α′B refer to the α′^{D} of the C-terminal domain. For “DBM” column, the letter (N) and (C), respectively, indicate the origin from the N-terminal domain and the C-terminal domain.

Optionally, some amino acid modifications can be further introduced in order to avoid the steric hindrance between amino acid side chains and/or to increase the stability. Optionally, some amino acid modifications can be further introduced in order to enhance the production and/or the solubility and to decrease the toxicity (Turmel et al, 1997, Nucleic Acod Research, 25, 2610-2619). Optionally, the loop can be completely or partially replaced by a convenient linker. A convenient linker is preferably flexible. Said flexible linker preferably comprises glycine, serine and threonine residues. Short flexible linker can also be introduced between the loop and the domains. Optionally, the loop can also be replaced by a loop from any other di-dodecapeptide meganuclease, preferably the I-Dmo I loop.

Optionally, such hybrid meganuclease comprising a first domain from the C-terminal domain of a di-dodecapeptide meganuclease (A) and a second domain from the C-terminal domain of another di-dodecapeptide meganuclease (B) can further comprise, at its N-terminal and/or C-terminal end, a loop or linker and any additional domain. Preferably, said additional domain is a DNA binding domain, a transcription activator or repressor domain, a nuclear localization signal or a DNA cleavage domain. Optionally, the endonuclease activity of such hybrid can be abolished.

A hybrid meganuclease comprising or consisting of a first domain derived from the C-terminal domain of a di-dodecapeptide meganuclease (A) and a second domain derived from the C-terminal domain of the same di-dodecapeptide meganuclease (A), said first and second domains being bound by a convenient linker, is also contemplated in the present invention. The same rules of design are applied to this kind of meganuclease.

Domain of Mono-Dodecapeptide Meganuclease/C-Terminal Domain of Di-Dodecapeptide Meganuclease

A fifth embodiment concerns a hybrid meganuclease comprising or consisting of a first domain from the domain of a mono-dodecapeptide meganuclease (A) and a second domain from the C-terminal domain of another di-dodecapeptide meganuclease (B), said first and second domains being bound by a convenient linker. The swapping point can be positioned from the last helix of the DNA binding moiety DBM to the beginning of the loop (LB) preceding the helix α′^{D}. It is preferably positioned at the beginning of the loop (LB) preceding the helix (α′^{D}). The resulting hybrid meganuclease comprises, from the N-terminal end to C-terminal end, the domain of the meganuclease A and the C-terminal domain of the meganuclease B. Such hybrid meganuclease schematically comprises:

| | V | | | | | | V′ |
|---|---|---|---|---|---|---|---|
| Type | optional | α^{D} | DBM | L | α′^{D} | DBM′ | optional |
| 1 | A | αA | A | B | α′B | B(C) | B |
| 2 | A | αA | A/B(N) | B | α′B | B(C) | B |

A and B indicating the meganuclease at the origin of the segment V, α^{D}, DBM, L, α′^{D}, DBM′, V′. A/B indicates that the swapping point is into the segment, the origin of the first part of the element is A and that of the second part is B. αA and αB refer to the α^{D} of the N-terminal domain and α′A and α′B refer to the α′^{D} of the C-terminal domain. For “DBM” column, the letter (N) and (C), respectively, indicate the origin from the N-terminal domain and the C-terminal domain.

In an alternative embodiment, the swapping point is replaced by a “swapping domain”. Indeed, instead of abruptly change the sequence, the helices α^{D} and α′^{D} of the hybrid meganuclease can be a mixture of the two initial meganucleases. The amino acid residues from the helices α^{D} and α′^{D} which are directed towards the helices interface are those of one meganuclease for both helices α^{D} and α′^{D} (those of α^{D} from the meganuclease A or those of α^{D} and α′^{D} from the meganuclease B). Optionally, the residues at the interface could be derived from another pair of dodecapeptide helices from a mono- or di-dodecapeptide meganuclease X. Within each domain, the amino acid residues from the helices α^{D} and α′^{D} which are directed towards the inside of the domain are those corresponding to the residues found at that position in that domain of the meganuclease it comes from (those of α^{D} from the meganuclease A and those of α′^{D} from the meganuclease B). Such hybrid meganuclease schematically comprises:

| | V op- | α^{D} | | | | α′^{D} | | | V′ |
|---|---|---|---|---|---|---|---|---|---|
| Type | tional | intra | inter | DBM | L | inter | intra | DBM′ | optional |
| 1 | A | αA | αA | A | B | αA | α′B | B(C) | B |
| 2 | A | αA | αB | A | B | α′B | α′B | B(C) | B |
| 3 | A | αA | αX | A | B | α′X | α′B | B(C) | B |
| 4 | A | αA | αX | A | B | αX | α′B | B(C) | B |
| 5 | A | αA | αA | A/ | B | αA | α′B | B(C) | B |
| | | | | B(N) | | | | | |
| 6 | A | αA | αB | A/ | B | α′B | α′B | B(C) | B |
| | | | | B(N) | | | | | |
| 7 | A | αA | αX | A/ | B | α′X | α′B | B(C) | B |
| | | | | B(N) | | | | | |
| 8 | A | αA | αX | A/ | B | αX | α′B | B(C) | B |
| | | | | B(N) | | | | | |

A, B, and X indicating the meganuclease at the origin of the segment V, α^{D}, DBM, L, α′^{D}, DBM′, V′. A/B indicates that the swapping point is into the segment, the origin of the first part of the element is A and that of the second part is B. “inter” refers to the residues of α^{D} and α′^{D} towards the interface between the domains, and “intra” refers to the residues of α^{D} and α′^{D} towards the inside of each domain. αA and αB refer to the α^{D} of the N-terminal domain and α′A and α′B refer to the α′^{D} of the C-terminal domain. For “DBM” column, the letter (N) and (C), respectively, indicate the origin from the N-terminal domain and the C-terminal domain.

Optionally, some amino acid modifications can be further introduced in order to avoid the steric hindrance between amino acid side chains and/or to increase the stability. Optionally, some amino acid modifications can be further introduced in order to enhance the production and/or the solubility and to decrease the toxicity (Turmel et al, 1997, Nucleic Acod Research, 25, 2610-2619). Optionally, the loop can be completely or partially replaced by a convenient linker A convenient linker is preferably flexible. Said flexible linker essentially comprises glycine, serine and threonine residues. Short flexible linker can also be introduced between the loop and the domains. Optionally, the loop can also be replaced by a loop from any other di-dodecapeptide meganuclease, preferably the I-Dmo I loop.

Optionally, such hybrid meganuclease comprising a first domain from the domain of a mono-dodecapeptide meganuclease (A) and a second domain from the C-terminal domain of another di-dodecapeptide meganuclease (B) can further comprise, at its N-terminal and/or C-terminal end, a loop or linker and any additional domain. Preferably, said additional domain is a DNA binding domain, a transcription activator or repressor domain, a nuclear localization signal or a DNA cleavage domain. Optionally, the endonuclease activity of such hybrid can be abolished.

C-Terminal Domain of Di-Dodecapeptide Meganuclease/N-Terminal Domain of Di-Dodecapeptide Meganuclease

A sixth embodiment concerns a hybrid meganuclease comprising or consisting of a first domain derived from the C-terminal domain of a di-dodecapeptide meganuclease (A) and a second domain derived from the N-terminal domain of another di-dodecapeptide meganuclease (B), said first and second domains being bound by a convenient linker. The first and the second domains are linked by either a convenient linker or a connecting loop from any di-dodecapeptide meganuclease Y, for example the loop of I-Dmo I meganuclease. A convenient linker is preferably flexible. Said flexible linker essentially comprises glycine, serine and threonine residues. Short flexible linkers can also be introduced between the loop and the domains. The linker is preferably attached at one end to the helix following the 4-stranded β-sheet of the DBM of the C-terminal domain of the meganuclease A and at the other end at the helix α^{D} of the N-terminal domain of the meganuclease B. The resulting hybrid meganuclease comprises, from the N-terminal end to C-terminal end, the C-terminal domain of the meganuclease A, a linker or a connecting loop and the N-terminal domain of the meganuclease B. Preferably, the resulting hybrid meganuclease does not comprise the DBM of the N-terminal domain of the meganuclease A, more preferably its N-terminal domain. Preferably, the resulting hybrid meganuclease does not comprise the DBM of the C-terminal domain of the meganuclease B, more preferably its C-terminal domain. Such hybrid meganuclease schematically comprises:

| V, optional | α^{D} | DBM | L | α′^{D} | DBM′ | V′, optional |
|---|---|---|---|---|---|---|
| A | α′A | A(C) | Y | αB | B(N) | B |

A, B and Y indicating the meganuclease at the origin of the segment V, α^{D}, DBM, L, α′^{D}, DBM′, V′. αA and αB refer to the α^{D} of the N-terminal domain and α′A and α′B refer to the α′^{D} of the C-terminal domain. For “DBM” column, the letter (N) and (C), respectively, indicate the origin from the N-terminal domain and the C-terminal domain.

In an alternative embodiment, the helices α^{D} and α′^{D} of the hybrid meganuclease can be a mixture of the two initial meganucleases. The amino acid residues from the helices α^{D} and α′^{D} which are directed Towards the helix interface are those of one meganuclease for both helices α^{D} and α′^{D} (those of α^{D} and α′^{D} from the meganuclease A or B).). Optionally, the residues at the interface could be derived from another pair of dodecapeptide helices from a mono- or di-dodecapeptide meganuclease X. Within each domain, the amino acid residues from the helices α^{D} and α′^{D} which are directed towards the inside of the domain are those corresponding to the residues found at that position in that domain of the meganuclease it comes from (those of α′^{D} from the meganuclease A and those of α^{D} from the meganuclease B). Such hybrid meganuclease schematically comprises

| | V, | | | | | | | | |
|---|---|---|---|---|---|---|---|---|---|
| | op- | α^{D} | | | | α′^{D} | | | V′, |
| Type | tional | intra | inter | DBM | L | inter | intra | DBM′ | optional |
| 1 | A | α′A | αA | A(C) | Y | α′A | αB | B(N) | B |
| 2 | A | α′A | αB | A(C) | Y | α′B | αB | B(N) | B |
| 3 | A | α′A | αX | A(C) | Y | α′X | αB | B(N) | B |
| 4 | A | α′A | αX | A(C) | Y | αX | αB | B(N) | B |

A, B, X and Y indicating the meganuclease at the origin of the segment V, α^{D}, DBM, L, α′^{D}, DBM′, V′. “inter” refers to the residues of α^{D} and α′^{D} towards the interface between the domains, and “intra” refers to the residues of α^{D} and α′^{D} towards the inside of each domain. αA and αB refer to the α^{D} of the N-terminal domain and α′A and α′B refer to the α′^{D} of the C-terminal domain. For “DBM” column, the letter (N) and (C), respectively, indicate the origin from the N-terminal domain and the C-terminal domain.

Optionally, some amino acid modifications can be further introduced in order to avoid the steric hindrance between amino acid side chains and/or to increase the stability. Optionally, some amino acid modifications can be further introduced in order to enhance the production and/or the solubility and to decrease the toxicity (Turmel et al, 1997, Nucleic Acod Research, 25, 2610-2619).

Optionally, such hybrid meganuclease comprising a first domain from the C-terminal domain of a di-dodecapeptide meganuclease (A) and a second domain from the N-terminal domain of another di-dodecapeptide meganuclease (B) can further comprise, at its N-terminal and/or C-terminal end, a loop or linker and any additional domain. Preferably, said additional domain is a DNA binding domain, a transcription activator or repressor domain, a nuclear localization signal or a DNA cleavage domain. Optionally, the endonuclease activity of such hybrid can be abolished.

A hybrid meganuclease comprising or consisting of a first domain derived from the C-terminal domain of a di-dodecapeptide meganuclease (A) and a second domain derived from the N-terminal domain of the same di-dodecapeptide meganuclease (A), said first and second domains being bound by a convenient linker, is also contemplated in the present invention. The same rules of design are applied to this kind of meganuclease.

Domain of Mono-Dodecapeptide Meganuclease/N-Terminal Domain of Di-Dodecapeptide Meganuclease

A seventh embodiment concerns a hybrid meganuclease comprising or consisting of a first domain derived from the domain of a mono-dodecapeptide meganuclease (A) and a second domain derived from the N-terminal domain of another di-dodecapeptide meganuclease (B), said first and second domains being bound by a convenient linker. The first and the second domains are linked by either a convenient linker or a connecting loop from any di-dodecapeptide meganuclease Y, for example the loop of I-Dmo I meganuclease. A convenient linker is preferably flexible. Said flexible linker essentially comprises glycine, serine and threonine residues. Short flexible linker can also be introduced between the loop and the domains. The linker is preferably attached at one end to the helix following the 4-stranded β-sheet of the DBM of the domain of the meganuclease A and at the other end at the helix α^{D} of the N-terminal domain of the meganuclease B. The resulting hybrid meganuclease comprises, from the N-terminal end to C-terminal end, the domain of the meganuclease A, a linker or a connecting loop and the N-terminal domain of the meganuclease B. Preferably, the resulting hybrid meganuclease does not comprise the DBM of the C-terminal domain of the meganuclease B, more preferably its C-terminal domain.

Such hybrid meganuclease schematically comprises:

| V, optional | α^{D} | DBM | L | α′^{D} | DBM′ | V′, optional |
|---|---|---|---|---|---|---|
| A | αA | A | Y | αB | B(N) | B |

A, B and Y indicating the meganuclease at the origin of the segment V, α^{D}, DBM, L, α′^{D}, DBM′, V′. αA and αB refer to the α^{D} of the N-terminal domain and α′A and α′B refer to the α′^{D} of the C-terminal domain. For “DBM” column, the letter (N) and (C), respectively, indicate the origin from the N-terminal domain and the C-terminal domain.

In an alternative embodiment, the helices α^{D} and α′^{D} of the hybrid meganuclease can be a mixture of the two initial meganucleases. The amino acid residues from the helices α^{D} and α′^{D} which are directed towards the helix interface are those of one meganuclease for both helices (those of α^{D} from the meganuclease A, or those of α^{D} and a′^{D} from the meganuclease B). Optionally, the residues at the interface could be derived from another pair of dodecapeptide helices from a mono- or di-dodecapeptide meganuclease X. Within each domain, the amino acid residues from the helices α^{D} and α′^{D} which are directed towards the inside of the domain are those corresponding to the residues found at that position in that domain of the meganuclease it comes from (those of α^{D} from the meganuclease A and those of α^{D} from the meganuclease B). Such hybrid meganuclease schematically comprises:

| | V, | | | | | | | | |
|---|---|---|---|---|---|---|---|---|---|
| | op- | α^{D} | | | | α′^{D} | | | V′, |
| Type | tional | intra | inter | DBM | L | inter | intra | DBM′ | optional |
| 1 | A | αA | αA | A | Y | αA | αB | B(N) | B |
| 2 | A | αA | αB | A | Y | α′B | αB | B(N) | B |
| 3 | A | αA | αX | A | Y | α′X | αB | B(N) | B |
| 4 | A | αA | αX | A | Y | αX | αB | B(N) | B |

A, B, X and Y indicating the meganuclease at the origin of the segment V, α^{D}, DBM, L, α′^{D}, DBM′, V′. “inter” refers to the residues of α^{D} and α′^{D} towards the interface between the domains, and “intra” refers to the residues of α^{D} and α′^{D} towards the inside of each domain. αA and αB refer to the α^{D} of the N-terminal domain and α′A and α′B refer to the α′^{D} of the C-terminal domain. For “DBM” column, the letter (N) and (C), respectively, indicate the origin from the N-terminal domain and the C-terminal domain.

Optionally, some amino acid modifications can be further introduced in order to avoid the steric hindrance between amino acid side chains and/or to increase the stability. Optionally, some amino acid modifications can be further introduced in order to enhance the production and/or the solubility and to decrease the toxicity (Turmel et al, 1997, Nucleic Acod Research, 25, 2610-2619).

Optionally, such hybrid meganuclease comprising a first domain from the domain of a mono-dodecapeptide meganuclease (A) and a second domain from the N-terminal domain of another di-dodecapeptide meganuclease (B) can further comprise, at its N-terminal and/or C-terminal end, a loop or linker and any additional domain. Preferably, said additional domain is a DNA binding domain, a transcription activator or repressor domain, a nuclear localization signal or a DNA cleavage domain. Optionally, the endonuclease activity of such hybrid can be abolished.

C-Terminal Domain of Di-Dodecapeptide Meganuclease/Domain of Mono-Dodecapeptide Meganuclease

A eighth embodiment concerns a hybrid meganuclease comprising or consisting of a first domain derived from the C-terminal domain of a di-dodecapeptide meganuclease (A) and a second domain derived from the domain of another mono-dodecapeptide meganuclease (B), said first and second domains being bound by a convenient linker The first and the second domains are linked by either a convenient linker or a connecting loop from any di-dodecapeptide meganuclease Y, for example the loop of I-Dmo I meganuclease. A convenient linker is preferably flexible. Said flexible linker essentially comprises glycine, serine and threonine residues. Short flexible linker can also be introduced between the loop and the domains. The linker is preferably attached at one end to the helix following the 4-stranded β-sheet of the DBM of the C-terminal domain of the meganuclease A and at the other end at the helix α^{D} of the domain of the meganuclease B. The resulting hybrid meganuclease comprises, from the N-terminal end to C-terminal end, the C-terminal domain of the meganuclease A, a linker or a connecting loop and the domain of the meganuclease B. Preferably, the resulting hybrid meganuclease does not comprise the DBM of the N-terminal domain of the meganuclease A, more preferably its N-terminal domain. Such hybrid meganuclease schematically comprises:

| V, optional | α^{D} | DBM | L | α′^{D} | DBM′ | V′, optional |
|---|---|---|---|---|---|---|
| A | α′A | A(C) | Y | αB | B | B |

A, B, and Y indicating the meganuclease at the origin of the segment V, α^{D}, DBM, L, α′^{D}, DBM′, V′. αA and αB refer to the α^{D} of the N-terminal domain and α′A and α′B refer to the α′^{D} of the C-terminal domain. For “DBM” column, the letter (N) and (C), respectively, indicate the origin from the N-terminal domain and the C-terminal domain.

In an alternative embodiment, the helices α^{D} and α′^{D} of the hybrid meganuclease can be a mixture of the two initial meganucleases. The amino acid residues from the helices α^{D} and α′^{D} which are directed towards the helix interface are those of one meganuclease for both helices (those of α^{D} and α′^{D} from the meganuclease A, or those of α^{D} from the meganuclease B). Optionally, the residues at the interface could be derived from another pair of dodecapeptide helices from a mono- or di-dodecapeptide meganuclease X. Within each domain, the amino acid residues from the helices α^{D} and α′^{D} which are directed towards the inside of the domain are those corresponding to the residues found at that position in that domain of the meganuclease it comes from (those of α′^{D} from the meganuclease A and those of α^{D} from the meganuclease B). Such hybrid meganuclease schematically comprises

| | V, | | | | | | | | |
|---|---|---|---|---|---|---|---|---|---|
| | op- | α^{D} | | | | α′^{D} | | | V′, |
| Type | tional | intra | inter | DBM | L | inter | intra | DBM′ | optional |
| 1 | A | α′A | αA | A(C) | Y | α′A | αB | B | B |
| 2 | A | α′A | αB | A(C) | Y | αB | αB | B | B |
| 3 | A | α′A | αX | A(C) | Y | αX | αB | B | B |
| 4 | A | α′A | αX | A(C) | Y | α′X | αB | B | B |

A, B, X and Y indicating the meganuclease at the origin of the segment V, α^{D}, DBM, L, α′^{D}, DBM′, V′. “inter” refers to the residues of α^{D} and α′^{D} towards the interface between the domains, and “intra” refers to the residues of α^{D} and α′^{D} towards the inside of each domain. αA and αB refer to the α^{D} of the N-terminal domain and α′A and α′B refer to the α′^{D} of the C-terminal domain. For “DBM” column, the letter (N) and (C), respectively, indicate the origin from the N-terminal domain and the C-terminal domain.

Optionally, some amino acid modifications can be further introduced in order to avoid the steric hindrance between amino acid side chains and/or to increase the stability. Optionally, some amino acid modifications can be further introduced in order to enhance the production and/or the solubility and to decrease the toxicity (Turmel et al, 1997, Nucleic Acod Research, 25, 2610-2619).

Optionally, such hybrid meganuclease comprising a first domain from the C-terminal domain of a di-dodecapeptide meganuclease (A) and a second domain from the domain of another mono-dodecapeptide meganuclease (B) can further comprise, at its N-terminal and/or C-terminal end, a loop or linker and any additional domain. Preferably, said additional domain is a DNA binding domain, a transcription activator or repressor domain, a nuclear localization signal or a DNA cleavage domain. Optionally, the endonuclease activity of such hybrid can be abolished.

Domain of Mono-Dodecapeptide Meganuclease/Domain of Mono-Dodecapeptide Meganuclease

A ninth embodiment concerns a hybrid meganuclease comprising or consisting of a first domain derived from the domain of a mono-dodecapeptide meganuclease (A) and a second domain derived from the domain of the same or another mono-dodecapeptide meganuclease (B), said first and second domains being bound by a convenient linker. The first and the second domains are linked by either a convenient linker or a connecting loop from any di-dodecapeptide meganuclease Y, for example the loop of I-Dmo I meganuclease. A convenient linker is preferably flexible. Said flexible linker essentially comprises glycine, serine and threonine residues. Short flexible linker can also be introduced between the loop and the domains. The linker is preferably attached at one end to the helix following the 4-stranded β-sheet of the DBM of the domain of the meganuclease A and at the other end at the helix α^{D} of the domain of the meganuclease B. The resulting hybrid meganuclease comprises, from the N-terminal end to C-terminal end, the domain of the meganuclease A deleted from the variable sequence VA located downstream of the DBM of B, a linker or a connecting loop and the domain of the meganuclease B. Such hybrid meganuclease schematically comprises:

| V, optional | α^{D} | DBM | L | α′^{D} | DBM′ | V′, optional |
|---|---|---|---|---|---|---|
| A | αA | A | Y | αB | B | B |

A, B and Y indicating the meganuclease at the origin of the segment V, α^{D}, DBM, L, α′^{D}, DBM′, V′. αA and αB refer to the α^{D} of the N-terminal domain and α′A and α′B refer to the α′^{D} of the C-terminal domain. For “DBM” column, the letter (N) and (C), respectively, indicate the origin from the N-terminal domain and the C-terminal domain.

In an alternative embodiment, the helices α^{D} and α′^{D} of the hybrid meganuclease can be a mixture of the two initial meganucleases. The amino acid residues from the helices α^{D} and α′^{D} which are directed towards the helix interface are those of one meganuclease for both helices (those of α^{D} from the meganuclease A or B). Optionally, the residues at the interface could be derived from another pair of dodecapeptide helices from a mono- or di-dodecapeptide meganuclease X. Within each domain, the amino acid residues from the helices α^{D} and α′^{D} which are directed towards the inside of the domain are those corresponding to the residues found at that position in that domain of the meganuclease it comes from (those of α^{D} from the meganuclease A and B). Such hybrid meganuclease schematically comprises:

| | V, | | | | | | | | |
|---|---|---|---|---|---|---|---|---|---|
| | op- | α^{D} | | | | α′^{D} | | | V′, |
| Type | tional | intra | inter | DBM | L | inter | intra | DBM′ | optional |
| 1 | A | αA | αA | A | Y | αA | αB | B | B |
| 2 | A | αA | αB | A | Y | αB | αB | B | B |
| 3 | A | αA | αX | A | Y | α′X | αB | B | B |
| 4 | A | αA | αX | A | Y | αX | αB | B | B |

A, B, Y and X indicating the meganuclease at the origin of the segment V, α^{D}, DBM, L, α′^{D}, DBM′, V′. “inter” refers to the residues of α^{D} and α′^{D} towards the interface between the domains, and “intra” refers to the residues of α^{D} and α′^{D} towards the inside of each domain. αA and αB refer to the α^{D} of the N-terminal domain and α′A and α′B refer to the α′^{D} of the C-terminal domain. For “DBM” column, the letter (N) and (C), respectively, indicate the origin from the N-terminal domain and the C-terminal domain.

Optionally, some amino acid modifications can be further introduced in order to avoid the steric hindrance between amino acid side chains and/or to increase the stability. Optionally, some amino acid modifications can be further introduced in order to enhance the production and/or the solubility and to decrease the toxicity (Turmel et al, 1997, Nucleic Acod Research, 25, 2610-2619).

Optionally, such hybrid meganuclease comprising a first domain from the domain of a mono-dodecapeptide meganuclease (A) and a second domain from the domain of another mono-dodecapeptide meganuclease (B) can further comprise, at its N-terminal and/or C-terminal end, a loop or linker and any additional domain. Preferably, said additional domain is a DNA binding domain, a transcription activator or repressor domain, a nuclear localization domain or a DNA cleavage domain. Optionally, the endonuclease activity of such hybrid can be abolished.

An example of hybrid meganuclease, more particularly a hybrid meganuclease comprising a first domain derived from the N-terminal domain of a di-dodecapeptide meganuclease (A) and a second domain derived from the C-terminal domain of another di-dodecapeptide meganuclease (B), is disclosed in example 1 for the I-Dmo I/I-Cre I hybrid meganuclease. An example of one way for introducing said linker between two domains is disclosed in example 2 for the single chain I-Cre I meganuclease.

Alternative engineering strategies are possible. For example, a flexible linker could be a sequence comprising a number of glycine, serine and threonine amino acids. A disadvantage, not present in our method, could be the need, in this case, to determine and eventually optimize for each domain combination the precise linker sequence and length, together with the connections to the protein domains.

Our preferred strategy is based on structural analyses and comparison of the parent proteins that are “domain swapped”. Another approach requires aligning the sequences of two proteins in the regions that most certainly correspond to motifs of conserved structures. For instance, sequence conservation of the dodecapeptide motifs is related to the persistent presence of an inter-domain two-helix bundle. Domain swapped endonucleases can be engineered by exchanging protein sequences somewhere within the second dodecapeptide motif, or directly prior to that motif where a linker region must be present.

The invention also contemplates the use of such hybrid meganuclease essentially as recognition domain. In this case, the endonuclease catalytic activity of the hybrid meganuclease can be abolished by some mutation, for example the acidic residues D/E which are necessary for the catalytic activity.

In one particular embodiment, the invention concerns a chimeric protein comprising one domain derived from a dodecapeptide meganuclease, a linker and an helix comprising the dodecapeptide motif. Optionally, said linker is a loop from a di-dodecapeptide meganuclease. Optionally, said chimeric protein further comprises an additional domain. Said additional domain is preferably a DNA binding domain, a transcription activator or repressor domain, a nuclear localization signal, or a DNA cleavage domain

### Single-Chain Meganucleases

In the present invention, we disclose a Single-chain meganuclease derived from “mono-dodecapeptide” meganucleases. The “mono-dodecapeptide” meganucleases are active as homodimer. Each monomer mainly dimerizes through their dodecapeptide motifs. This single-chain meganuclease covalently binds two monomers of a “mono-dodecapeptide” meganuclease modified such as to introduce a covalent link between the two sub-units of this enzyme. Preferably, the covalent link is introduced by creating a peptide bond between the two monomers. However, others convenient covalent link are also contemplated by the present invention. The invention concerns a single-chain meganuclease comprising a first and a second domain in the orientation N-terminal toward C-terminal, wherein said first and second domains are derived from the same mono-dodecapeptide meganuclease and wherein said single-chain meganuclease is capable of causing DNA cleavage. Same principles of hybrid meganucleases apply to the single chain meganuclease. More particularly, see hybrid meganuclease comprising a first domain derived from the domain of a mono-dodecapeptide meganuclease (A) and a second domain derived from the domain of the same or another mono-dodecapeptide meganuclease (B).

The single-chain meganuclease can comprise two sub-units from the same meganuclease such as single-chain I-Cre and single-chain I-Ceu I. The single-chain I-Ceu II is also contemplated by the invention. The invention concerns a single chain meganuclease of I-Cre comprising the sequence of SEQ ID N^{o} 6. A single-chain meganuclease has multiple advantages. For example, a single-chain meganuclease is easier to manipulate. The single-chain meganuclease is thermodynamically favored, for example for the recognition of the target sequence, compared to a dimer formation. The single-chain meganuclease allows the control of the oligomerisation.

The making of a single-chain version of I-CreI (scI-CreI) is described in example 2.

In this first version, the N-terminal domain of the single-chain meganuclease (positions 1 to 93 of I-CreI amino acid sequence) consisted essentially of the αββαββα a fold (core domain) of an I-CreI monomer whereas the C-terminal (positions 8 to 163 of I-CreI amino acid sequence) was a nearly complete I-CreI monomer. The linker (MLERIRLFNMR; SEQ ID NO: 17) was derived from the loop joining the two domains of I-DmoI.

Although the first scI-CreI was a functional meganuclease, it was less stable than I-CreI, probably due a less optimal folding as compared to its natural counterpart. The design of the first scI-CreI matched the structure of double LAGLIDADG endonucleases and therefore differed from that of I-CreI in that the N-terminal domain is shorter than the C-terminal domain and lacks the C-terminal subdomain made of three α-helices which may be present in the C-terminal domain of some double LAGLIDADG endonucleases.

The inventors thought it could be further improved because the removal of the three C-terminal helices from the first monomer might affect the folding, stability and consequently the cleavage activity of the first scI-CreI since recent works have shown the crucial role of the C-terminal subdomain of I-CreI in the protein DNA binding properties and DNA target cleavage activity (Prieto et al., Nucleic Acids Res., 2007), 35, 3267-3271).

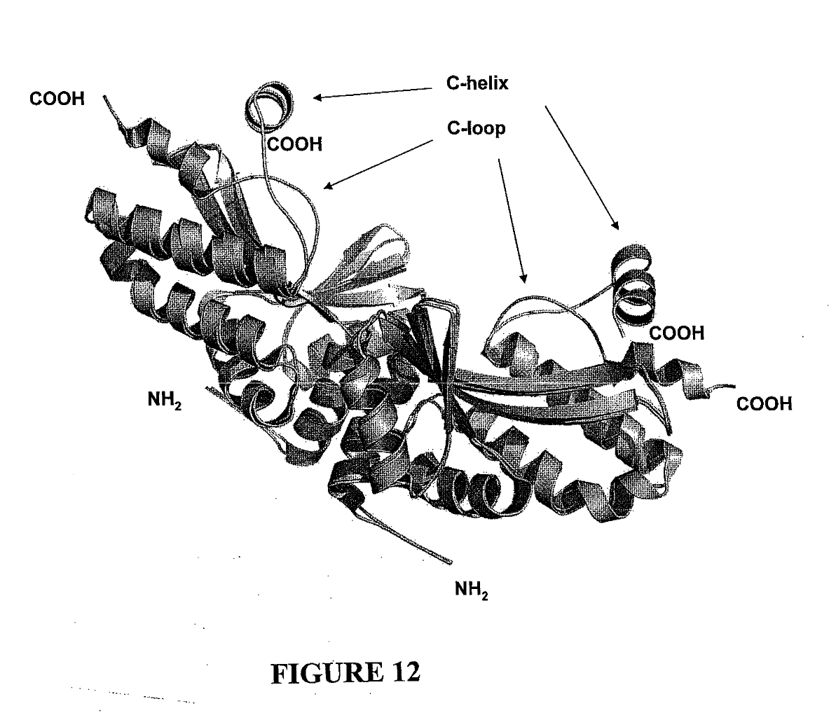

However, the three C-terminal helices terminate at opposite sides of the dimer structure of I-CreI, far away from the N-terminal helices comprising the LAGLIDADG motif (FIG. 12). The length of a flexible linker connecting the C-terminal residue of one domain to the N-terminal residue of the other domain (end to end fusion) would be considerable. Besides engineering such linker would be difficult due to the necessity to go across a large part of the protein surface. Therefore, it is uncertain that proper packing be obtained.

Here the inventors present a new way to design a single chain molecule derived from the I-CreI homodimeric meganuclease. This strategy preserves the core αββαββα (also named as α₁β₁β₂α₂β₃β₄α₃) fold as well as the C-terminal part of the two linked I-CreI units.

This design greatly decreases off-site cleavage and toxicity while enhancing efficacy. The structure and stability of this single; chain molecule are very similar to those of the heterodimeric variants and this molecule appears to be monomeric in solution. Moreover, the resulting proteins trigger highly efficient homologous gene targeting (at the percent level) at an endogenous locus in human cells (SCID gene) in living cells while safeguarding more effectively against potential genotoxicity. In all respects, this single-chain molecule performs as well as I-SceI, the monomeric homing endonuclease considered to be gold standard in terms of specificity. These properties place this new generation of meganucleases among the best molecular scissors available for genome surgery strategies and should facilitate gene correction therapy for monogenetic diseases, such as for example severe combined immunodeficiency (SCID), while potentially avoiding the deleterious effects of previous gene therapy approaches.

The subject matter of the present invention is a single-chain I-CreI meganuclease (scI-CreI) comprising two domains (N-terminal and C-terminal) joined by a peptidic linker, wherein:

(a) each domain, derived from a parent I-CreI monomer, comprises a portion of said parent I-CreI monomer which extends at least from the beginning of the first alpha helix (α₁) to the end of the C-terminal loop of I-CreI and includes successively: the α₁β₁β₂α₂β₃β₄α₃ core domain, the α₄ and α₅ helices and the C-terminal loop of I-CreI, and

(b) the two domains are joined by a peptidic linker which allows said two domains to fold as a I-CreI dimer that is able to bind and cleave a chimeric DNA target comprising one different half of each parent homodimeric I-CreI meganuclease target sequence.

The single-chain I-CreI meganuclease according to the invention is also named, scI-CreI meganuclease or sc-I-CreI.

According to the present invention, the sequence of the linker is chosen so as to allow the two domains of the sc-I-CreI to fold as a I-CreI dimer and to bind and cleave a chimeric DNA target comprising one different half of each parent homodimeric I-CreI meganuclease target sequence.

I-CreI dimer formation may be assayed by well-known assays such as sedimentation equilibrium experiments performed by analytical centrifugation, as previously described in Prieto et al., Nucleic Acids Research, 2007, 35, 3267-3271.

DNA binding may be assayed by well-known assays such as for example, electrophoretic mobility shift assays (EMSA), as previously described in Prieto et al., Nucleic Acids Research, 2007, 35, 3267-3271.

The cleavage activity of the single-chain meganuclease according to the invention may be measured by any well-known, in vitro or in vivo cleavage assay, such as those described in Example 3 or in the International PCT Application WO 2004/067736; Epinat et al., Nucleic Acids Res., 2003, 31, 2952-2962; Chames et al., Nucleic Acids Res., 2005, 33, e178; Arnould et al., J. Mol. Biol., 2006, 355, 443-458; Arnould et al., J. Mol. Biol., Epub 10 May 2007.

For example, the cleavage activity of the single-chain meganuclease according to the present invention may be measured by a direct repeat recombination assay, in yeast or mammalian cells, using a reporter vector, by comparison with that of the corresponding heterodimeric meganuclease or of another single-chain meganuclease, derived from identical parent I-CreI monomers. The reporter vector comprises two truncated, non-functional copies of a reporter gene (direct repeats) and the genomic (non-palindromic) DNA target sequence—comprising one different half of each (palindromic or pseudo-palindromic) parent homodimeric I-CreI meganuclease target sequence, within the intervening sequence, cloned in a yeast or a mammalian expression vector. Expression of the meganuclease results in cleavage of the genomic chimeric DNA target sequence. This cleavage induces homologous recombination between the direct repeats, resulting in a functional reporter gene (LacZ, for example), whose expression can be monitored by appropriate assay. In addition, the activity of the single-chain meganuclease towards its genomic DNA target can be compared to that of I-CreI towards the I-CreI site, at the same genomic locus, using a chromosomal assay in mammalian cells (Arnould et al., J. Mol. Biol., Epub 10 May 2007). In addition the specificity of the cleavage by the single-chain meganuclease may be assayed by comparing the cleavage of the chimeric DNA target sequence with that of the two palindromic sequences cleaved by the parent I-CreI homodimeric meganucleases.

The N-terminal sequence of the two domains of the sc-I-CreI may start at position 1, 2, 3, 4, 5, 6 or 8 of I-CreI. In a preferred embodiment, the N-terminal domain starts at position 1 or 6 of I-CreI and the C-terminal domain starts at position 2 or 6 of I-CreI.

The C-terminal sequence of the two domains terminates just after the C-terminal loop, for example at position 143 or 145 of I-CreI. Alternatively, the C-terminal sequence of the domain(s) further includes at least the alpha 6 helix (positions 145 to 150 of I-CreI) and eventually additional C-terminal residues. In this case, the C-terminal sequence of the domain(s) may terminates at position 150, 151, 152, 153, 154, 155, 156, 157, 158, 159, 160, 161, 162 or 163 of I-CreI. Preferably at position 152 to 160. For example at position, 152, 156, 160 or 163. In a preferred embodiment, the N-terminal domain terminates at position 163 of I-CreI. More preferably, both domains terminate at positions 163 of I-CreI.

In another preferred embodiment of the sc-I-CreI meganuclease, the linker comprises or consists of a linear sequence of 10 to 100 consecutive amino acids, preferably 15 to 50 amino acids, more preferably 20 to 35 amino acids, even more preferably 25 to 35 amino acids. The sequence of the linker is predicted to form an helix, either an alpha-helix (prediction made with SOPMA—Self-Optimized Prediction Method with Alignment—Geourjon and Deléage, 1995) or a type II polypro line helix. An example of such polyproline helix is represented by SEQ ID NO: 31 or 32 (Table 1). More preferably, the linker comprises an alpha-helix. The linker is advantageously selected from the group consisting of the sequences comprising or consisting of SEQ ID NO: 18 to 35. Preferably, it is selected from the group consisting of the sequences of SEQ ID NO: 18 to 28 and 30 to 35. More preferably, the linker is selected from the group consisting of the sequences SEQ ID NO: 18, 20 to 24, 27, 32, 34 and 35. Even more preferably, the linker consists of the sequence SEQ ID NO: 18.

The single-chain I-CreI meganuclease according to the invention may be derived from wild-type I-CreI monomers or functional variants thereof. In addition, one or more residues inserted at the NH₂ terminus and/or COOH terminus of the parent monomers. Additional codons may be added at the 5′ or 3′ end of the parent monomers to introduce restrictions sites which are used for cloning into various vectors. An example of said sequence is SEQ ID NO: 39 which has an alanine (A) residue inserted after the first methionine residue and an alanine and an aspartic acid (AD) residues inserted after the C-terminal proline residue. These sequences allow having DNA coding sequences comprising the NcoI (ccatgg) and EagI (cggccg) restriction sites which are used for cloning into various vectors. For example, a tag (epitope or polyhistidine sequence) may also be introduced at the NH₂ terminus of the N-terminal domain and/or COOH terminus of C-terminal domain; said tag is useful for the detection and/or the purification of said sc-I-CreI meganuclease.

Preferably, the sc-I-CreI meganuclease is derived from the monomers of a heterodimeric I-CreI variant, more preferably of a variant having novel cleavage specificity as previously described (Arnould et al., J. Mol. Biol., 2006, 355, 443-458; Smith et al., Nucleic Acids Res., 2006, 34, e149; Arnould et al., J. Mol. Biol., Epub 10 May 2007; International PCT Applications WO 2006/097784, WO 2006/097853, WO 2007/049095, WO 2007/057781, WO 2007/060495, WO 2007/049156, WO 2007/093836 and WO 2007/093918)

Therefore the domains of the single-chain I-CreI meganuclease may comprise mutations at positions of I-CreI amino acid residues that contact the DNA target sequence or interact with the DNA backbone or with the nucleotide bases, directly or via a water molecule; these residues are well-known in the art (Jurica et al., Molecular Cell., 1998, 2, 469-476; Chevalier et al., J. Mol. Biol., 2003, 329, 253-269). Preferably said mutations modify the cleavage specificity of the meganuclease and result in a meganuclease with novel specificity, which is able to cleave a DNA target from a gene of interest. More preferably, said mutations are substitutions of one or more amino acids in a first functional subdomain corresponding to that situated from positions 26 to 40 of I-CreI amino acid sequence, that alter the specificity towards the nucleotide at positions ±8 to 10 of the DNA target, and/or substitutions in a second functional subdomain corresponding to that situated from positions 44 to 77 of I-CreI amino acid sequence, that alter the specificity towards the nucleotide at positions ±3 to 5 of the DNA target, as described previously (International PCT Applications WO 2006/097784, WO 2006/097853, WO 2007/060495, WO 2007/049156, WO 2007/049095 and WO 2007/057781; Arnould et al., J. Mol. Biol., 2006, 355, 443-458; Smith et al., Nucleic Acids Res., 2006). The substitutions correspond advantageously to positions 26, 28, 30, 32, 33, 38, and/or 40, 44, 68, 70, 75 and/or 77 of I-CreI amino acid sequence. For cleaving a DNA target, wherein n₋₄ is t or n₊₄ is a, said I-CreI domain has advantageously a glutamine (Q) in position 44; for cleaving a DNA target, wherein n₋₄ is a or n₊₄ is t, said domain has an alanine (A) or an asparagine in position 44, and for cleaving a DNA target, wherein n₋₉ is g or n₊₉ is c, said domain has advantageously an arginine (R) or a lysine (K) in position 38.

According to another preferred embodiment of said scI-CreI meganuclease, at least one domain has mutations at positions 26 to 40 and/or 44 to 77 of I-CreI, said scI-CreI meganuclease being able to cleave a non-palindromic DNA sequence, wherein at least the nucleotides at positions +3 to +5, +8 to +10, −10 to −8 and −5 to −3 of said DNA sequence correspond to the nucleotides at positions +3 to +5, +8 to +10, −10 to −8 and −5 to −3 of a DNA target from a gene of interest. Preferably, both domains of the scI-CreI meganuclease are mutated at positions 26 to 40 and/or 44 to 77. More preferably, both domains have different mutations at positions 26 to 40 and 44 to 77 of I-CreI.

In another preferred embodiment of said scI-CreI meganuclease, at least one domain comprises one or more mutations at positions of other amino acid residues of I-CreI that interact with the DNA target sequence. In particular, additional substitutions may be introduced at positions contacting the phosphate backbone, for example in the final C-terminal loop (positions 137 to 143; Prieto et al., Nucleic Acids Res., 2007, 35, 3262-3271). Preferably said residues are involved in binding and cleavage of said DNA cleavage site. More preferably, said residues are at positions 138, 139, 142 or 143 of I-CreI. Two residues may be mutated in one domain provided that each mutation is in a different pair of residues chosen from the pair of residues at positions 138 and 139 and the pair of residues at positions 142 and 143. The mutations which are introduced modify the interaction(s) of said amino acid(s) of the final C-terminal loop with the phosphate backbone of the I-CreI site. Preferably, the residue at position 138 or 139 is substituted by an hydrophobic amino acid to avoid the formation of hydrogen bonds with the phosphate backbone of the DNA cleavage site. For example, the residue at position 138 is substituted by an alanine or the residue at position 139 is substituted by a methionine. The residue at position 142 or 143 is advantageously substituted by a small amino acid, for example a glycine, to decrease the size of the side chains of these amino acid residues. More preferably, said substitution in the final C-terminal loop modifies the specificity of the scI-CreI meganuclease towards the nucleotide at positions ±1 to 2, ±6 to 7 and/or ±11 to 12 of the I-CreI site.

In another preferred embodiment of said scI-CreI meganuclease, at least one domain comprises one or more additional mutations that improve the binding and/or the cleavage properties, including the cleavage activity and/or specificity of the scI-CreI meganuclease towards the DNA target sequence from a gene of interest. The additional residues which are mutated may be on the entire I-CreI sequence.

According to a more preferred embodiment of said sc-I-CreI meganuclease, said additional mutation(s) impair(s) the formation of functional homodimers from the domains of the sc-I-CreI meganuclease.

Each parent monomer has at least two residues Z and Z′ of the dimerisation interface which interact with residues Z′ and Z, respectively of the same or another parent monomer (two pairs ZZ′ of interacting residues) to form two homodimers and one heterodimer. According to the present invention, one of the two pairs of interacting residues of the dimerisation interface is swapped to obtain a first domain having two residues Z or Z′ and a second domain having two residues Z′ or Z, respectively. As a result, the first and the second domains each having two residues Z or two residues Z′ can less easily homodimerize (inter-sc-I-CreI domains interaction) than their parent counterpart, whereas the presence of two pairs ZZ′ of interacting residues at the interface of the two domains of the sc-I-CreI makes the heterodimer formation (intra-sc-I-CreI domains interaction) favourable.

Therefore the domains of the sc-I-CreI meganuclease have advantageously at least one of the following pairs of mutations, respectively for the first (N-terminal or C-terminal domain) and the second domain (C-terminal domain or N-terminal domain):

a) the substitution of the glutamic acid in position 8 with a basic amino acid, preferably an arginine (first domain) and the substitution of the lysine in position 7 with an acidic amino acid, preferably a glutamic acid (second domain); the first domain may further comprise the substitution of at least one of the lysine residues in positions 7 and 96, by an arginine.

b) the substitution of the glutamic acid in position 61 with a basic amino acid, preferably an arginine (first domain) and the substitution of the lysine in position 96 with an acidic amino acid, preferably a glutamic acid (second domain); the first domain may further comprise the substitution of at least one of the lysine residues in positions 7 and 96, by an arginine

c) the substitution of the leucine in position 97 with an aromatic amino acid, preferably a phenylalanine (first domain) and the substitution of the phenylalanine in position 54 with a small amino acid, preferably a glycine (second domain); the first domain may further comprise the substitution of the phenylalanine in position 54 by a tryptophane and the second domain may further comprise the substitution of the leucine in position 58 or lysine in position 57, by a methionine, and

d) the substitution of the aspartic acid in position 137 with a basic amino acid, preferably an arginine (first domain) and the substitution of the arginine in position 51 with an acidic amino acid, preferably a glutamic acid (second domain).

For example, the first domain may have the mutation D137R and the second domain, the mutation R51D.

Alternatively, the sc-I-CreI meganuclease comprises at least two pairs of mutations as defined in a), b) c) or d), above; one of the pairs of mutation is advantageously as defined in c) or d). Preferably, one domain comprises the substitution of the lysine residues at positions 7 and 96 by an acidic amino acid (D or E) and the other domain comprises the substitution of the glutamic acid residues at positions 8 and 61 by a basic amino acid (K or R). More preferably, the sc-I-CreI meganuclease comprises three pairs of mutations as defined in a), b) and c), above. The sc-I-CreI meganuclease consists advantageously of a first domain (A) having at least the mutations selected from: (i) E8R, E8K or E8H, E61R, E61K or E61H and L97F, L97W or L97Y; (ii) K7R, E8R, E61R, K96R and L97F, or (iii) K7R, E8R, F54W, E61R, K96R and L97F and a second domain (B) having at least the mutations (iv) K7E or K7D, F54G or F54A and K96D or K96E; (v) K7E, F54G, L58M and K96E, or (vi) K7E, F54G, K57M and K96E.

Another example of mutations that impair the formation of functional homodimers from the domains of the sc-I-CreI meganuclease is the G19S mutation. The G19S mutation is advantageously introduced in one of the two domains of the sc-I-CreI meganuclease, so as to obtain a single-chain meganuclease having enhanced cleavage activity and enhanced cleavage specificity. In addition, to enhance the cleavage specificity further, the other domain or both domains may carry distinct mutation(s) that impair the formation of a functional homodimer or favors the formation of the heterodimeric sc-I-CreI meganuclease, as defined above. For example, one monomer comprises the G19S mutation and the K7E and K96E or the E8K and E61R mutations and the other monomer comprises the E8K and E61R or the K7E and K96E mutations, respectively.

In another preferred embodiment of said sc-I-CreI meganuclease, said mutations are replacement of the initial amino acids with amino acids selected from the group consisting of: A, D, E, G, H, K, N, P, Q, R, S, T, Y, C, V, L and W.

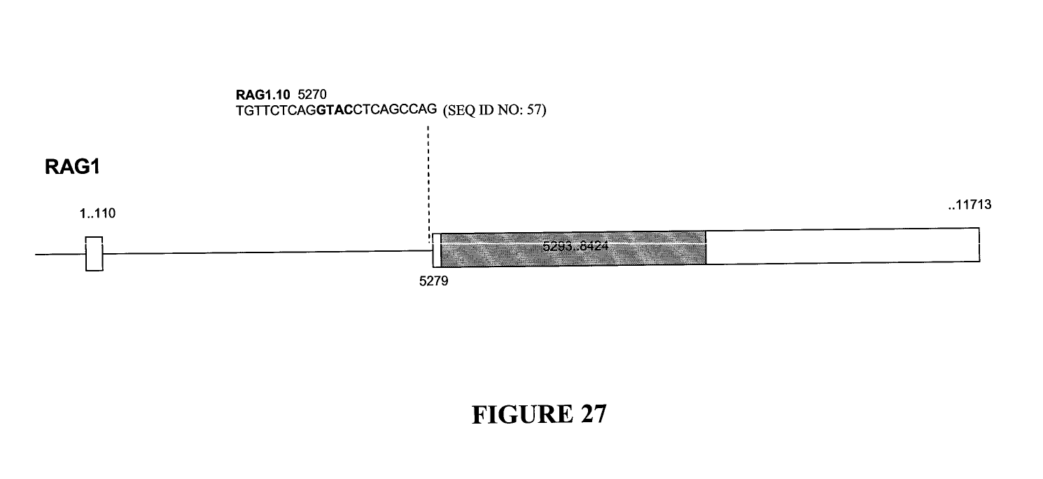

The subject-matter of the present invention is also a sc-I-CreI meganuclease comprising the sequence SEQ ID NO: 111 or 113; these sc-I-CreI meganucleases cleave the RAG1 target (RAG1.10; SEQ ID NO: 57 and FIG. 27) which is present in the human RAG1 gene. This sc-I-CreI meganuclease can be used for repairing RAG1 mutations associated with a SCID syndrome or for genome engineering at the RAG1 gene loci. Since RAG1.10 is upstream of the coding sequence (FIG. 27), these sc-I-CreI meganucleases can be used for repairing any mutation in the RAG1 gene by exon knock-in, as described in the International PCT Application WO 2008/010093).

The invention also relates to variants of the hybrid or single chain meganuclease according to the present invention. Preferably, the variants of hybrid or single chain meganuclease comprise a core of the meganuclease consisting of the two domains and the linker having at least 70% of identity with the initial hybrid or single chain meganuclease, more preferably at least 80, 90, 95 or 99% of identity. The variant may be 1) one in which one or more of the amino acid residues are substituted with a conserved or non-conserved amino acid residue and such substituted amino acid residue may or may not be one encoded by the genetic code, or 2) one in which one or more of the amino acid residues includes a substituent group, or 3) one in which the hybrid or single chain meganuclease is fused with another compound, such as a compound to increase the half-life of the polypeptide (for example, polyethylene glycol), or 4) one in which the additional amino acids are fused to the hybrid meganuclease, such as a leader or secretory sequence or a sequence which is employed for purification of the hybrid meganuclease. Such variants are deemed to be within the scope of those skilled in the art. In the case of an amino acid substitution in the amino acid sequence of a hybrid or single chain meganuclease according to the invention, one or several amino acids can be replaced by “equivalent” amino acids. The expression “equivalent” amino acid is used herein to designate any amino acid that may be substituted for one of the amino acids having similar properties, such that one skilled in the art of peptide chemistry would expect the secondary structure and hydropathic nature of the polypeptide to be substantially unchanged. Generally, the following groups of amino acids represent equivalent changes: (1) Ala, Pro, Gly, Glu, Asp, Gln, Asn, Ser, Thr; (2) Cys, Ser, Tyr, Thr; (3) Val, Ile, Leu, Met, Ala, Phe; (4) Lys, Arg, His; (5) Phe, Tyr, Trp, His. A modified hybrid or single chain meganuclease is a peptide molecule which is resistant to proteolysis, a peptide in which the —CONH-peptide bond is modified and replaced by a (CH2NH) reduced bond, a (NHCO) retro inverso bond, a (CH2-O) methylene-oxy bond, a (CH2-S) thiomethylene bond, a (CH2CH2) carba bond, a (CO—CH2) cetomethylene bond, a (CHOH—CH2) hydroxyethylene bond), a (N—N) bound, a E-alcene bond or also a —CH═CH— bond. The invention also encompasses a hybrid or single chain meganuclease in which at least one peptide bound has been modified as described above.

The present invention concerns any cell or non-human animal comprising a hybrid or single chain meganuclease according to the present invention. The present invention also comprises any pharmaceutical composition comprising a hybrid or single chain meganuclease according to the present invention.

### Polynucleotides Encoding Hybrid and Single Chain Meganucleases, Vectors, Cells and Animals

The present invention concerns a recombinant polynucleotide encoding a hybrid or single chain meganuclease according to the present invention. Among these polynucleotides, the invention concerns a polynucleotide comprising a sequence selected from the group consisting of SEQ ID N^{o} 1, 3 and 5. According to a preferred embodiment of said polynucleotide, the nucleic acid sequences encoding the two I-CreI domains of the single-chain meganuclease have less than 80% nucleic sequence identity, preferably less than 70% nucleic sequence identity. This reduces the risk of recombination between the two sequences and as a result, the genetic stability of the polynucleotide construct and the derived vector is thus increased. This may be obtained by rewriting the I-CreI sequence using the codon usage and the genetic code degeneracy. For example, one of the domains is derived from the wild-type I-CreI coding sequence (SEQ ID NO: 38) and the other domain is derived from a rewritten version of the I-CreI coding sequence (SEQ ID NO: 40). Furthermore the codons of the cDNAs encoding the single-chain meganuclease are chosen to favour the expression of said proteins in the desired expression system. The present invention concerns:

a) any vector comprising a polynucleotide sequence encoding a hybrid or single chain meganuclease according to the present invention;

b) any prokaryotic or eukaryotic cell comprising either a polynucleotide sequence encoding a hybrid or single chain meganuclease according to the present invention or said vector comprising said polynucleotide sequence;

c) any non-human transgenic animal or transgenic plant comprising a polynucleotide sequence encoding a hybrid or single chain meganuclease according to the present invention, a vector comprising said polynucleotide, or a cell comprising either said polynucleotide or a vector comprising said polynucleotide.

The vector comprising a polynucleotide encoding a hybrid or single chain meganuclease contains all or part of the coding sequence for said a hybrid or single chain meganuclease operably linked to one or more expression control sequences whereby the coding sequence is under the control of transcriptional signals to permit production or synthesis of said hybrid or single chain meganuclease. Therefore, said polynucleotide encoding a hybrid or single chain meganuclease is comprised in an expression cassette. More particularly, the vector comprises a replication origin, a promoter operatively linked to said encoding polynucleotide, a ribosome-binding site, an RNA-splicing site (when genomic DNA is used), a polyadenylation site and a transcription termination site. It also can comprise an enhancer. Selection of the promoter will depend upon the desired route for expressing the hybrid or single chain meganuclease. Suitable promoters include tissue specific and/or inducible promoters. Examples of inducible promoters are: eukaryotic metallothionine promoter which is induced by increased levels of heavy metals, prokaryotic lacZ promoter which is induced in response to isopropyl-β-D-thiogalacto-pyranoside (IPTG) and eukaryotic heat shock promoter which is induced by increased temperature. Examples of tissue specific promoters are skeletal muscle creatine kinase, prostate-specific antigen (PSA), α-antitrypsin protease, human surfactant (SP) A and B proteins, β-casein and acidic whey protein genes.

The invention concerns a method for producing a hybrid or single chain meganuclease comprising introducing an expression vector into a cell compatible with the element of said expression vector.

The polynucleotide sequence encoding the hybrid or single chain meganuclease can be prepared by any method known.

For example, the polynucleotide sequence encoding the hybrid or single chain meganuclease can be prepared from the polynucleotide sequences encoding the initial meganucleases by usual molecular biology technologies.

Preferably the polynucleotide sequence encoding the hybrid or single chain meganuclease is preferably generated by well-known back or reverse-translation methods. Number of back-translation softs are available, for example in the GCG sequence analysis package (University of Wisconsin, Madison, Wis.); in DNA strider, in EMBOSS (European Molecular Biology Open Software Suite http://www.hgmp.mrc.ac.uk/Software/EMBOSS/Apps/backtranseq.html); etc. . . . ). The obtained polynucleotide sequence can be synthesized through any method well known by the man skilled in the art.

Methods for making I-CreI variants having novel cleavage specificity have been described previously (Chames et al., Nucleic Acids Res., 2005, 33, e178, and Arnould et al., J. Mol. Biol., 2006, 355, 443-458; Smith et al., Nucleic Acids Res., 2006, 34, e149; Arnould et al., J. Mol. Biol., Epub 10 May 2007; International PCT Applications WO 2004/067736, WO 2006/097784, WO 2006/097853, WO 2007/049095, WO 2007/057781, WO 2007/060495, WO 2007/049156, WO 2007/093836, WO 2007/093918, WO2008/010009, WO2008/010093, WO2008/059317, WO2008/059382, WO2008/093152, WO2008/093249, WO2008/02198, WO2008/02199, WO2008/02274, WO2008/149176, WO2008/152523, WO2008/152524 WO2009/001159, WO2009013559, WO2009013622, WO2009019528 and WO2009019614). The single-chain meganuclease of the invention may be derived from I-CreI or functional variants thereof by using well-known recombinant DNA and genetic engineering techniques. For example, a sequence comprising the linker coding sequence in frame with, either the 3′ end of the N-terminal domain coding sequence or the 5′ end of the C-terminal domain coding sequence is amplified from a DNA template, by polymerase chain reaction with specific primers. The PCR fragment is then cloned in a vector comprising the sequence encoding the other domain by using appropriate restriction sites.

### Hybrid Recognition and Cleavage Sites

The hybrid meganucleases according to the present invention recognize and cleave a hybrid site or target comprising the half sites recognized by each domains comprised in the hybrid meganuclease.

The recognition sites of the meganucleases are not palyndromic. For a di-dodecapeptide meganuclease A, the N-terminal and C-terminal domains recognize two different half recognition sites; herein called “Site L_{A}” and “Site R_{A}”. The indicated sequences for the initial half sites generally correspond to the sequence of the strand + of the genome. “L” refers to the left part of the site and “R” to the right part. Therefore, the site of the meganuclease A can be described as Site L_{A}-Site R_{A}. By “RC Site” is intended in the present invention the reverse complementary sequence of the half site on the + strand of the genomic target site.

The orientation of the parental molecules onto their respective recognition and cleavage site is not always well defined or known. Thus, preferably, several half-site combinations have to be synthesized and subjected to cleavage. For example, for an hybrid meganuclease comprising a first domain derived from a domain of a di-dodecapeptide meganuclease A and a second domain derived from a domain of a di-dodecapeptide meganuclease B, the following target sites will be preferably used;

Site L_{A}-Site R_{B}

RC Site R_{A}-Site R_{B}

Site L_{A}-RC Site L_{B}

RC Site R_{A}-RC Site L_{B}.

When the orientation is known, the hybrid site can be easily defined without any combination. The determination of the orientation can be determined by the generation of hybrid meganuclease and the study of their specificity on the several targets of the above mentioned combination. See example 3 (I-Dmo I/I-Cre I) for a way of determination of the respective orientation of the meganuclease and the recognition site.

In order to test the endonuclease activity and specificity, synthetic target site corresponding to the fusion of parental half sites or a combination thereof is synthesized and cloned into a vector or use as such.

The invention concerns an isolated or recombinant polynucleotide comprising a hybrid target site according to the present invention. This hybrid target site comprises two half sites from the initial meganucleases, one per meganuclease. The invention also concerns a vector comprising a hybrid target site according to the present invention. The invention further concerns a cell comprising a recombinant polynucleotide or a vector comprising a hybrid target site according to the present invention. The invention further concerns a non-human animal comprising a recombinant polynucleotide or a vector comprising a hybrid target site according to the present invention. The invention further concerns a plant comprising a recombinant polynucleotide or a vector comprising a hybrid target site according to the present invention.

When nothing is known about the recognition and cleavage site, the following method could be applied in order to define this site.

Homing intron-encoded meganuclease of the dodecapeptide family, recognize and cleave normally a sequence present in a gene without intron, said intron comprising the encoding sequence for the meganuclease. In fact, the target sequence for the meganuclease is represented by the junction of upstream and downstream exon, naturally present in the gene without intron. The double strand break, for this class of meganuclease, occurs inside the recognition site.

In the absence of data on the extension of the recognition site, the length of the recognition site should be 30 nucleotides on the left part (upstream exon) and 30 nucleotides on the right side (downstream exon) meaning 60 nucleotides to test the binding and/or the cleavage of the protein. The recognition sequence should be centered around the position of the intron insertion point in the gene without intron.

In the particular case of inteins (PI), the canonical target should be represented by the junction, at the DNA level, of the sequence encoding the two extein and should be of equivalent size of the previous site (about 30+30=60 nucleotides total). In the absence of data on the recognition site, one difference with the determination of the site of intron-encoded meganucleases, could be the presence of a cystein codon (observed in a large number of cases) in the middle of the recognition sequence (TGT or TGC).

In the case of hybrid-meganucleases, the canonical target sequence, should be represented by the junction between the two half-site of each original meganuclease.

### In Vitro Cleavage Assay

The recognition and cleavage of a specific DNA sequence by the hybrid and/or single chain meganucleases according to the present invention can be assayed by any method known by the man skilled in the art. One way to test the activity of the hybrid and/or single-chain meganuclease is to use an in vitro cleavage assay on a polynucleotide substrate comprising the recognition and cleavage site corresponding to the assayed meganuclease. Said polynucleotide substrate is a synthetic target site corresponding to the fusion of parental half sites which is synthesized and cloned into a vector. This vector, once linearized by a restriction enzyme, and then incubated with the hybrid. Said polynucleotide substrate can be linear or circular and comprises preferably only one cleavage site. The assayed meganuclease is incubated with the polynucleotide substrate in appropriate conditions. The resulting polynucleotides are analyzed by any known method, for example by electrophoresis on agarose or by chromatography. The meganuclease activity is detected by the apparition of two bands (products) and the disappearance of the initial full-length substrate band. Preferably, said assayed meganuclease is digested by proteinase K, for example, before the analysis of the resulting polynucleotides. In one embodiment, the target product is prepared with the introduction of a polynucleotide comprising the recognition and cleavage sequence in a plasmid by TA or restriction enzyme cloning, optionally following by the linearization of the plasmid. Preferably, such linearization is not done in the surrounding of the target site. See Wang et al, 1997, Nucleic Acid Research, 25, 3767-3776; In materials & Methods “I-CreI endonuclease activity assays” section, the disclosure of which is incorporated herein by reference) and the characterization papers of the considering meganucleases.

The orientation of the initial meganuclease onto their respective recognition and cleavage site is not always known (for example, does Nterm I-DmoI bind the left or right half part of its site?). Thus, several half-site combinations have to be synthesized and subjected to cleavage.

### In Vivo Cleavage Assay

The recognition and cleavage of a specific DNA sequence by the hybrid and/or single-chain meganucleases according to the present invention can be assayed by any method known by the man skilled in the art. One way to test the activity of the hybrid and/or single-chain meganuclease is to use an in vivo a Single-strand annealing recombination test (SSA). This kind of test is known by the man skilled in the art and disclosed for example in Rudin et al (Genetics 1989, 122, 519-534); Fishman-Lobell & Haber (Science 1992, 258, 480-4); Lin et al (Mol. Cell. Biol., 1984, 4, 1020-1034) and Rouet et al (Proc. Natl. Acad. Sci. USA, 1994, 91, 6064-6068); the disclosure of which is incorporated herein by reference. This test could be applied for assaying any endonuclease, preferably rare-cutting endonuclease.

To test the hybrid and/or single-chain meganucleases according to the present invention, we developed an in vivo assay based on SSA in an eukaryotic cell, namely a yeast cell or a higher eukaryotic cell such as mammalian cells. In one preferred embodiment of the in vivo assay, the method uses a yeast cell. This organism has the advantage recombine naturally its DNA via homologous recombination with a high frequency.

This in vivo test is based on the reparation by SSA of a reporter marker induced by site-specific double-stand break generated by the assayed meganuclease at its recognition and cleavage site. The target consists of a modified reporter gene with an internal duplication separated by a intervening sequence comprising the recognition and cleavage site of the assayed meganuclease. The internal duplication should contain at least 50 bp, preferably at least 200 bp, more preferably at least 300 or 400 bp. The efficiency of the SSA test will be increased by the size of the internal duplication. The intervening sequences at and preferably less than 2 kb. Preferably, the size of the intervening sequence, which comprises least the recognition and cleavage site, is between few by to 1 kb, more preferably among 500 bp. The intervening sequence can optionally comprise a selection marker (for example, neomycin phosphotransferase, histidinol dehydrogenase, dihydrofolate reductase, hygromycin phosphotransferase, herpes simplex virus thymidine kinase, adenosine deaminase, glutamine synthetase, and hypoxanthine-guanine phosphoribosyl transferase for eukaryotic cell culture; TRP1 or URA3 for S. cerevisiae; tetracycline, rifampicin or ampicillin resistance in E. coli; etc. . . . ). By reporter gene is intended any nucleic acid encoding a product easily assayed, for example β-galactosidase, luciferase, alkaline phosphatase, green fluorescent protein, tyrosinase, DsRed proteins. The reporter gene is preferably operably linked to a constitutive promoter relating to the cell used in the assay (for example CMV promoter). According to the present assay method, the reporter will be detected only if a SSA event occurs following the double-strand break introduced by the assayed meganuclease.

The assayed meganuclease is introduced in an expression cassette. Preferably this expression cassette is on a separate construct. The meganuclease encoding sequence can be operably linked to an inducible promoter or to a constitutive promoter. Of course, the promoter needs to be compatible with the cell used in the assay. In a preferred embodiment, the construct is comprised by a plasmid.

Optionally, each construct can comprise a selectable marker to ensure the presence of the plasmid in the cell. The presence of this selectable marker is required for the assay proceeded in yeast cell. For example, for yeast, the first construct comprising the target gene can comprise a Leu2 selectable marker allowing transformed yeast to grow on a synthetic medium that does not contain any Leucine and the second construct can comprise the Trp1 selectable marker allowing transformed yeast to grow on a synthetic medium that does not contain any tryptophane.

The two constructs are used to transform simultaneously an appropriate cell. If the meganuclease is expressed and recognizes its cleavage site on the reporter construct, it can promote double-stand break and site-specific recombination between the target sequences by a mechanism known as Single-Strand Annealing. The resulting reporter gene should then express fully active reporter protein. Control experiments can be done with construct that does not express any meganuclease gene and reporter construct with no recognition and cleavage site.

The example 4 disclosed the use of a target consisting of a modified beta-galactosidase gene with a 900 pb internal duplication separated by the Ura3 selectable marker and a cleavage site for the assayed meganuclease (single-chain I-Cre I).

The recognition and cleavage by the hybrid and/or single-chain meganucleases according to the present invention can be also assayed with a gene conversion assay. For example, the reporter construct comprises a first modified reporter gene with a deletion and an insertion of an intervening sequence at the place of the deletion. This intervening sequence comprises the recognition and cleavage site of the assayed meganuclease. The reporter construct further comprises the fragment of the reporter gene which has been deleted flanked at each side by the reporter gene sequences bordering the deletion. The bordering sequences comprises at least 100 bp of homology with the reporter gene at each side, preferably at least 300 pb. The induction of a site-specific double-stand break generated by the assayed meganuclease at its recognition and cleavage site will trigger to a gene conversion event resulting in a functional reporter gene. This kind of assay are documented in the following articles: Rudin et al (Genetics 1989, 122, 519-534), Fishman-Lobell & Haber (Science 1992, 258, 480-4), Paques & Haber (Mol. Cell. Biol., 1997, 17, 6765-6771), the disclosures of which are incorporated herein by reference.

Otherwise, the recognition and cleavage by the hybrid and/or single-chain meganucleases according to the present invention can be assayed through a recombination assay on chromosomic target. The recombination can be based on SSA or gene conversion mechanisms. For example, a mutated non-functional reporter gene comprising a recognition and cleavage site for the assayed meganuclease is introduced into the chromosome of the cell. Said cleavage site has to be in the vicinity of the mutation, preferably at less than 1 kb from the mutation, more preferably at less than 500 bp, 200 bp, or 100 pb surrounding the mutation. By transfecting the cell with a fragment of the functional reporter gene corresponding to the mutation and an expression construct allowing the production of the assayed meganuclease in the cell, the repair by homologous recombination of the double-strand break generated by the assayed meganuclease will lead to a functional reporter gene, said reporter gene expression being detected. This kind of assay is documented in the following articles: Rouet et al (Mol. Cell. Biol., 1994, 14, 8096-8106); Choulika et al (Mol. Cell. Biol., 1995, 15, 1968-1973); Donoho et al (Mol. Cell. Biol., 1998, 18, 4070-4078); the disclosures of which are incorporated herein by reference.

### Search of Hybrid Meganuclease for a Target Gene or Virus

The present invention discloses new methods to discover novel targets for natural or hybrids of meganucleases in a targeted locus of a virus genome and other genomes of interest, more particularly in a gene thereof. Indeed, from the large number of LAGLIDADG meganuclease, the hybrid meganucleases allow to generate a high diversity of target sites. These new target sites are rare in the genome of interest, preferably almost unique, and are useful for several applications further detailed below.

A database comprising all known meganucleases target sites is prepared. Preferably, such database comprises the target sites that have been experimentally shown to be cleaved by a meganuclease. A second database is designed comprising all possible targets for hybrid meganucleases. Such targets for hybrid meganucleases can be designed as disclosed above.

From these databases, an alignment is done to find homologous sequences without gaps, said homologous sequences having an identity of at least 70%, preferably 80%, more preferably 90%, still more preferably 95%. Various alignment algorithms and/or programs may be used to calculate the identity between two sequences, including FASTA, or BLAST which are available as a part of the GCG sequence analysis package (University of Wisconsin, Madison, Wis.), and can be used with, e.g., default settings.

### Use of Meganucleases

The hybrid meganuclease according to the present invention can be used for molecular biology and for genetic engineering and gene therapy, more particularly according to the methods described in WO 96/14408, U.S. Pat. No. 5,830,729, WO 00/46385, WO 00/46386 and provisional application filed on 26/10/01 under Docket N^{o} 3665-20 and on 14/09/01 under Docket N^{o} 3665-17; the disclosure of these documents being incorporated by reference.

Molecular biology includes with no limitations, DNA restriction and DNA mapping. Genetic and genome engineering for non therapeutic purposes include for example (i) gene targeting of specific loci in cell packaging lines for protein production, (ii) gene targeting of specific loci in crop plants, for strain improvements and metabolic engineering, (iii) targeted recombination for the removal of markers in genetically modified crop plants, (iv) targeted recombination for the removal of markers in genetically modified microorganism strains (for antibiotic production for example).

A very interesting use of the hybrid meganuclease is for targeting a particular genomic locus of interest. Indeed, the induction of a double stranded break at a site of interest in chromosomal DNA of the cell is accompanied by the introduction of a targeting DNA homologous to the region surrounding the cleavage site with a high efficient. The hybrid meganuclease can be used for targeting a particular genomic locus comprising the hybrid target site. Hybrid meganucleases according to the present invention can be used in methods involving the induction in cells of double stranded DNA cleavage at a specific site in chromosomal DNA, thereby inducing a DNA recombination event, a DNA loss or cell death. For more detailed, see WO 96/14408, U.S. Pat. No. 5,830,729, WO 00/46386. The hybrid meganuclease can be used in a method of genetic engineering comprising the following steps: 1) introducing a double-stranded break at the genomic locus comprising the hybrid target site with the corresponding hybrid meganuclease; 2) providing a targeting DNA construct comprising the sequence to introduce flanked by homologous sequence to the targeting locus. Indeed, the homologous DNA is at the left and right arms of the targeting DNA construct and the DNA which modifies the sequence of interest is located between the two arms. Preferably, homologous sequences of at least 50 bp, preferably more than 100 by and more preferably more than 200 by are used. Therefore, the targeting DNA construct is preferably from 200 pb to 6000 pb, more preferably from 1000 pb to 2000 pb. Indeed, shared DNA homologies are located in regions flanking upstream and downstream the site of the break and the DNA sequence to be introduced should be located between the two arms. The sequence to be introduced is preferably a sequence which repairs a mutation in the gene of interest (gene correction or recovery of a functional gene), for the purpose of genome therapy. Alternatively, it can be any other sequence used to alter the chromosomal DNA in some specific way including a sequence used to modify a specific sequence, to restore a functional gene in place of a mutated one, to attenuate or activate the gene of interest, to inactivate or delete the gene of interest or part thereof, to introduce a mutation into a site of interest or to introduce an exogenous gene or part thereof. Such chromosomal DNA alterations are used for genome engineering (animal models/human recombinant cell lines).

Said hybrid meganuclease can be provided to the cell either through an expression vector comprising the polynucleotide sequence encoding said hybrid meganuclease and suitable for its expression in the used cell or the hybrid meganuclease itself. According to another advantageous embodiment of said vector, it includes a targeting DNA construct comprising sequences sharing homologies with the region surrounding the genomic DNA cleavage site as defined above. Alternatively, the vector coding for the single-chain meganuclease and the vector comprising the targeting DNA construct are different vectors. The hybrid or single chain meganucleases according to the present invention can be used for the deletion of a viral genome or a part thereof. Indeed, a cut in the viral genome induces a recombination that can lead to the deletion of a part or the whole viral genome deletion. This method is generally called virus pop-out. Therefore, the hybrid meganucleases allow the targeting of the viral genome. See WO 96/14408 example 5. Therefore, the invention concerns a method of deleting a viral genome or a part thereof, wherein a double-strand break in the viral genome is induced by a meganuclease according to the present invention and said double-strand break induces a recombination event leading to the deletion of the viral genome or a part thereof.

For the determination of the relevant hybrid meganuclease in order to introduce a double-strand cleavage at a target locus or a target viral genome, see section the immediate previous section.

The subject-matter of the present invention is also a method for preventing, improving or curing a disease caused by an infectious agent that presents a DNA intermediate, in an individual in need thereof, said method comprising at least the step of administering to said individual a meganuclease as defined above, by any means.

The subject-matter of the present invention is also the use of at least one meganuclease as defined above, one polynucleotide, preferably included in an expression vector, as defined above, in vitro, for inhibiting the propagation, inactivating or deleting an infectious agent that presents a DNA intermediate, in biological derived products or products intended for biological uses or for disinfecting an object.

The subject-matter of the present invention is also a method for decontaminating a product or a material from an infectious agent that presents a DNA intermediate, said method comprising at least the step of contacting a biological derived product, a product intended for biological use or an object, with a meganuclease as defined above, for a time sufficient to inhibit the propagation, inactivate or delete said infectious agent.

In a particular embodiment, said infectious agent is a virus. For example said virus is an adenovirus (Ad11, Ad21), herpesvirus (HSV, VZV, EBV, CMV, herpesvirus 6, 7 or 8), hepadnavirus (HBV), papovavirus (HPV), poxvirus or retrovirus (HTLV, HIV).

In another use, the hybrid and single chain meganucleases can be for in vivo excision of a polynucleotide fragment flanked by at least one, preferably two, hybrid target site. Hybrid meganucleases according to the present invention can be used in methods involving the excision of targeting DNA or polynucleotide fragment from a vector within cells which have taken up the vector. Such methods involve the use of a vector comprising said polynucleotide fragment flanked by at least one, preferably two, hybrid target site and either an expression vector comprising a polynucleotide encoding the hybrid and single chain meganuclease corresponding to the hybrid target site suitable for the expression in the used cell, or said hybrid and single chain meganuclease. Said excised polynucleotide fragment can be used for transgenesis as described in detail in provisional application filed on 26/10/01 under U.S. Ser. No. 60/330,639 and on 14/09/01 under U.S. Ser. No. 60/318,818. Said excised targeting DNA comprises homologous DNA at the left and right arms of the targeting DNA and the DNA which modifies the sequence of interest is located between the two arms. For more detail, see WO 00/46385. Said method of excision of targeting DNA from a vector within the cell can be used for repairing a specific sequence of interest in chromosomal DNA, for modifying a specific sequence or a gene in chromosomal DNA, for attenuating an endogeneous gene of interest, for introducing a mutation in a target site, or for treating or prohylaxis of a genetic disease in an individual.

The subject-matter of the present invention is also a method of genetic engineering, characterized in that it comprises a step of double-strand nucleic acid breaking in a site of interest located on a vector comprising a DNA target as defined hereabove, by contacting said vector with a meganuclease as defined above, thereby inducing an homologous recombination with another vector presenting homology with the sequence surrounding the cleavage site of said meganuclease.

The subject-matter of the present invention is also a method of genome engineering, characterized in that it comprises the following steps: 1) double-strand breaking a genomic locus comprising at least one DNA target of a meganuclease as defined above, by contacting said target with said meganuclease; 2) maintaining said broken genomic locus under conditions appropriate for homologous recombination with a targeting DNA construct comprising the sequence to be introduced in said locus, Harked by sequences sharing homologies with the targeted locus.

The subject-matter of the present invention is also a method of genome engineering, characterized in that it comprises the following steps: 1) double-strand breaking a genomic locus comprising at least one DNA target of a meganuclease as defined above, by contacting said cleavage site with said meganuclease; 2) maintaining said broken genomic locus under conditions appropriate for homologous recombination with chromosomal DNA sharing homologies to regions surrounding the cleavage site.

The subject-matter of the present invention is also the use of at least one meganuclease as defined above, one or two derived polynucleotide(s), preferably included in expression vector(s), as defined above, for the preparation of a medicament for preventing, improving or curing a genetic disease in an individual in need thereof, said medicament being administrated by any means to said individual.

The subject-matter of the present invention is also a method for preventing, improving or curing a genetic disease in an individual in need thereof, said method comprising the step of administering to said individual a composition comprising at least a meganuclease as defined above, by any means.

In this case, the use of the meganuclease as defined above, comprises at least the step of (a) inducing in somatic tissue(s) of the individual a double stranded cleavage at a site of interest of a gene comprising at least one recognition and cleavage site of said meganuclease, and (b) introducing into the individual a targeting DNA, wherein said targeting DNA comprises (1) DNA sharing homologies to the region surrounding the cleavage site and (2) DNA which repairs the site of interest upon recombination between the targeting DNA and the chromosomal DNA. The targeting DNA is introduced into the individual under conditions appropriate for introduction of the targeting DNA into the site of interest.

According to the present invention, said double-stranded cleavage is induced, either in toto by administration of said meganuclease to an individual, or ex vivo by introduction of said meganuclease into somatic cells removed from an individual and returned into the individual after modification.

In a preferred embodiment of said use, the meganuclease is combined with a targeting DNA construct comprising a sequence which repairs a mutation in the gene flanked by sequences sharing homologies with the regions of the gene surrounding the genomic DNA cleavage site of said meganuclease, as defined above. The sequence which repairs the mutation is either a fragment of the gene with the correct sequence or an exon knock-in construct.

For correcting a gene, cleavage of the gene occurs in the vicinity of the mutation, preferably, within 500 by of the mutation. The targeting construct comprises a gene fragment which has at least 200 by of homologous sequence flanking the genomic DNA cleavage site (minimal repair matrix) for repairing the cleavage, and includes the correct sequence of the gene for repairing the mutation. Consequently, the targeting construct for gene correction comprises or consists of the minimal repair matrix; it is preferably from 200 pb to 6000 pb, more preferably from 1000 pb to 2000 pb.

For restoring a functional gene, cleavage of the gene occurs upstream of a mutation. Preferably said mutation is the first known mutation in the sequence of the gene, so that all the downstream mutations of the gene can be corrected simultaneously. The targeting construct comprises the exons downstream of the genomic DNA cleavage site fused in frame (as in the cDNA) and with a polyadenylation site to stop transcription in 3′. The sequence to be introduced (exon knock-in construct) is flanked by introns or exons sequences surrounding the cleavage site, so as to allow the transcription of the engineered gene (exon knock-in gene) into a mRNA able to code for a functional protein. For example, the exon knock-in construct is flanked by sequences upstream and downstream.

The subject-matter of the present invention is also a composition characterized in that it comprises at least one meganuclease, one polynucleotide, preferably included in an expression vector, as defined above.

In a preferred embodiment of said composition, it comprises a targeting DNA construct comprising the sequence which repairs the site of interest flanked by sequences sharing homologies with the targeted locus as defined above. Preferably, said targeting DNA construct is either included in a recombinant vector or it is included in an expression vector comprising the polynucleotide encoding the meganuclease, as defined in the present invention.

The subject-matter of the present invention is also products containing at least a meganuclease, one expression vector encoding said meganuclease, and a vector including a targeting construct, as defined above, as a combined preparation for simultaneous, separate or sequential use in the prevention or the treatment of a genetic disease.

For purposes of therapy, the meganuclease and a pharmaceutically acceptable excipient are administered in a therapeutically effective amount. Such a combination is said to be administered in a “therapeutically effective amount” if the amount administered is physiologically significant. An agent is physiologically significant if its presence results in a detectable change in the physiology of the recipient. In the present context, an agent is physiologically significant if its presence results in a decrease in the severity of one or more symptoms of the targeted disease and in a genome correction of the lesion or abnormality.

In one embodiment of the uses according to the present invention, the meganuclease is substantially non-immunogenic, i.e., engenders little or no adverse immunological response. A variety of methods for ameliorating or eliminating deleterious immunological reactions of this sort can be used in accordance with the invention. In a preferred embodiment, the meganuclease is substantially free of N-formyl methionine. Another way to avoid unwanted immunological reactions is to conjugate meganucleases to polyethylene glycol (“PEG”) or polypropylene glycol (“PPG”) (preferably of 500 to 20,000 daltons average molecular weight (MW)). Conjugation with PEG or PPG, as described by Davis et al. (U.S. Pat. No. 4,179,337) for example, can provide non-immunogenic, physiologically active, water soluble endonuclease conjugates with anti-viral activity. Similar methods also using a polyethylene-polypropylene glycol copolymer are described in Saifer et al. (U.S. Pat. No. 5,006,333).

The meganuclease can be used either as a polypeptide or as a polynucleotide construct/vector encoding said polypeptide. It is introduced into cells, in vitro, ex vivo or in vivo, by any convenient means well-known to those in the art, which are appropriate for the particular cell type, alone or in association with either at least an appropriate vehicle or carrier and/or with the targeting DNA. Once in a cell, the meganuclease and if present, the vector comprising targeting DNA and/or nucleic acid encoding a meganuclease are imported or translocated by the cell from the cytoplasm to the site of action in the nucleus.

The meganuclease (polypeptide) may be advantageously associated with: liposomes, polyethyleneimine (PEI), and/or membrane translocating peptides (Bonetta, The Scientist, 2002, 16, 38; Ford et al., Gene Ther., 2001, 8, 1-4; Wadia and Dowdy, Curr. Opin. Biotechnol., 2002, 13, 52-56); in the latter case, the sequence of the meganuclease fused with the sequence of a membrane translocating peptide (fusion protein).

Vectors comprising targeting DNA and/or nucleic acid encoding a meganuclease can be introduced into a cell by a variety of methods (e.g., injection, direct uptake, projectile bombardment, liposomes, electroporation). Meganucleases can be stably or transiently expressed into cells using expression vectors. Techniques of expression in eukaryotic cells are well known to those in the art. (See Current Protocols in Human Genetics: Chapter 12 “Vectors For Gene Therapy” & Chapter 13 “Delivery Systems for Gene Therapy”). Optionally, it may be preferable to incorporate a nuclear localization signal into the recombinant protein to be sure that it is expressed within the nucleus.

The present invention also relates to the resulting cells and to their uses, such as for production of proteins or other gene products or for treatment or prophylaxis of a condition or disorder in an individual (e.g. a human or other mammal or vertebrate) arising as a result of a genetic defect (mutation). For example, cells can be produced (e.g., ex vivo) by the methods described herein and then introduced into an individual using known methods. Alternatively, cells can be modified in the individual (without being removed from the individual).

The invention also relates to the generation of animal models of disease in which hybrid meganuclease sites are introduced at the site of the disease gene for evaluation of optimal delivery techniques.

The subject-matter of the present invention is also the use of at least one meganuclease, as defined above, as a scaffold for making other meganucleases. For example other rounds of mutagenesis and selection/screening can be performed on the single-chain meganuclease, for the purpose of making novel homing endonucleases.

The uses of the meganuclease and the methods of using said meganucleases according to the present invention include also the use of the polynucleotide, vector, cell, transgenic plant or non-human transgenic mammal encoding said meganuclease, as defined above.

According to another advantageous embodiment of the uses and methods according to the present invention, said meganuclease, polynucleotide, vector, cell, transgenic plant or non-human transgenic mammal are associated with a targeting DNA construct as defined above. Preferably, said vector encoding the meganuclease, comprises the targeting DNA construct, as defined above.

### EXAMPLES

### Example 1

### I-Dmo I/I-Cre I Hybrid Meganuclease

I-DmoI/I-Cre I Hybrid Meganuclease Design

I-DmoI is a thermostable endonuclease encoded by an intron in the 23S rRNA gene of the hyperthermophilic archaeon Desulfurococcus mobilis (Dalgaard et al., 1993, Proc Natl Acad Sci USA, 90, 5414-5417). I-DmoI belongs to the LAGLIDADG family of meganucleases. Its structure, solved by X-ray crystallography (pdb code 1b24) (Silva et al., 1999, J Mol Biol, 286, 1123-1136), consists of two similar α/β domains (αββαββα) related by pseudo two-fold symmetry. A dodecapeptide motif is located at the C-terminal end of the first α-helix in each domain. These helices form a two-helix bundle at the interface between the domains and are perpendicular to a saddle-shaped DNA binding surface formed by two four-stranded antiparallel β-sheets (FIG. 1).

I-CreI is another LAGLIDADG meganuclease, encoded by an open reading frame contained within a self-splicing intron in the Chlamydomonas reinhardtii chloroplast 23S rRNA gene (Durrenberger and Rochaix, 1991, Embo J, 10, 3495-3501). However, unlike most members of this protein family, I-CreI contains a single copy of the dodecapeptide motif, and functions in a dimeric form. I-CreI dimers display the overall architecture of single chain LAGLIDADG proteins (FIG. 2) (Chevalier et al., 2001, Nat Struct Biol, 8, 312-316; Heath et al., 1997, Nat Struct Biol, 4, 468-476; Jurica et al., 1998, Mol Cell, 2, 469-476). Each monomer corresponds to a single domain, providing one of the two four-stranded antiparallel β-sheets for DNA-binding, and the dodecapeptide motifs are within α-helices at the inter-domain interface.

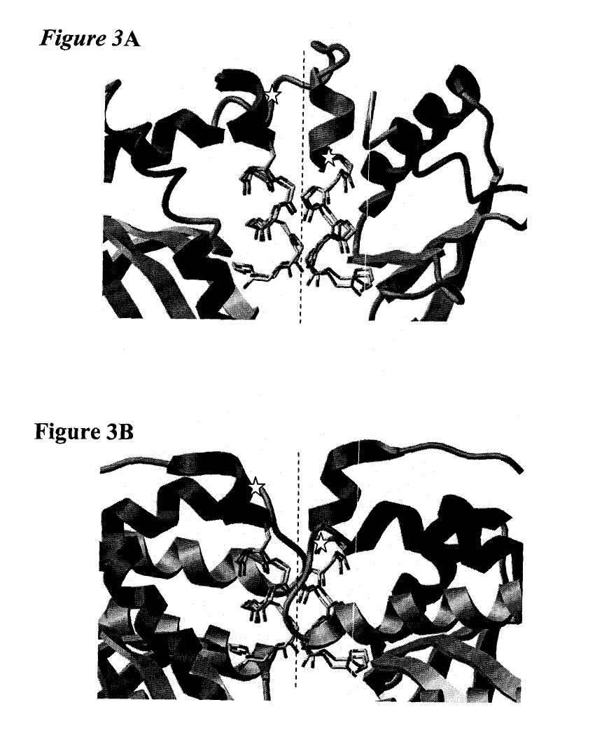

As they display similar overall topology, the structures of I-DmoI and I-CreI can be superimposed to low (local) root mean square deviation (RMSD), each monomer from I-CreI finely matching one of the two domains from I-DmoI. An optimal structural match (RMSD=0.66 Å, FIG. 3) was obtained superimposing the following atoms (residue numeration corresponds to either pdb structures, and for I-CreI, residues in the second monomer are shifted by 200 with respect to those in the first monomer):

| Source residues | Target residues | Atoms superimposed |
|---|---|---|
| I-DmoI, 14-22 | I-CreI, 13-21 | N, Cα, C, O |
| I-DmoI, 110-118 | I-CreI, 213-221 | N, Cα, C, O |
| I-DmoI, Tyr13 | I-CreI, Tyr12 | N, Cα, C, O, Cβ, Cγ |
| I-DmoI, Phe109 | I-CreI, Tyr212 | N, Cα, C, O, Cβ, Cγ |

The low RMSD is a strong indication of the similarity of the two dodecapeptide inter-domain packing interfaces. The dodecapeptide motifs and corresponding α-helices aligned sequences are:

| Motifs | Sequences |
|---|---|
| I-DmoI, 1^{st} dodecapeptide α- | SGISAY₁₃ LLGLIIGDG |
| helix | |
| I-DmoI, 2^{nd} dodecapeptide α- | EQIAF₁₀₉ IKGLYVAEG |
| helix | |
| I-CreI, dodecapeptide α-helix | NKIEFLLY₁₂ LAGFVDGDG |
| (1^{st} monomer) | |
| I-CreI, dodecapeptide α-helix | NKEFLLY₂₁₂ LAGFVDGDG |
| (2^{nd} monomer) | |

Visual inspection of the superimposition indicated that a hybrid of both proteins may be formed, by replacing the second I-DmoI domain with the corresponding I-CreI monomer. The I-DmoI sequence, starting from some point (the swap point) within the loop that connects both DNA binding domains or within the second dodecapeptide α-helix that follows, should be replaced by that of I-CreI starting at a corresponding point. We chose to swap the domains at the beginning of the second dodecapeptide motif. The resulting hybrid protein sequence was the protein sequence of I-DmoI up to Phe109 (included) and the protein sequence of I-CreI from Leu213 (included). Residues from I-CreI that precede Leu213 were thus removed. See FIG. 6A for the amino acid (SEQ ID N^{o} 2) and polynucleotide sequences (SEQ ID N^{o} 1) of such hybrid.

In a modeled structure, the novel inter-domain packing interface presented only little defects, e.g. no amino acid side chain has steric clashes that should not easily relax, except perhaps for Ile107 (I-DmoI) that has overlaps with Phe294 (I-CreI). In order to suppress the resulting potentially unfavorable repulsion, the isoleucine residue is replaced by a leucine amino acid (the aligned amino acid residue in I-CreI). Eventually, the inter-domain linker sequence is as follows:

| I-DmoI | Linker (I-DmoI) | I-CreI |
|---|---|---|
| (N- | NMLERIRLFNMREQLAF | LAGF . . . (C- |
| term) . . . YYFA | SEQ ID N^{o} 7 | term) |

Furthermore, Leu47, His51 and Leu55 (I-DmoI) are too close to Lys296 (I-CreI). This is uncertain, however, as distortion of the protein main chain structure (the I-DmoI region where residues 47, 51 and 55 are located) may indicate the structure is not fully reliable. Nevertheless, a second version of the hybrid protein is designed, which includes three additional mutations: L47A, H51A and L55D. Choices of alanine amino acids were made to stabilize the α-helical conformation of the corresponding residues. The third mutation (acid aspartic) should lead to the formation of an inter-domain salt-bridge to Lys296, thereby providing added stabilization. See FIG. 6B for the amino acid (SEQ ID N^{o} 4) and polynucleotide sequences (SEQ ID N° 3) of such hybrid.

Hybrid I-Dmo I/I-Cre I Meganuclease Production

### Solutions

Sonication solution: 25 mM HEPES (pH 7.5), 5% (v/v) glycerol, 0.1% (v/v) anti-proteases solution;

Solution A: 25 mM HEPES (pH 7.5), 5% (v/v) glycerol,

Storage solution: 25 mM HEPES (pH 7.5), 20% (v/v) glycerol

Standard reaction solution: 12.5 mM HEPES (pH 7.5), 2.5% (v/v) glycerol, 5-10 mM MgCl₂;

Standard stop solution (10×): 0.1 M Tris-HCl pH 7.5, 0.25 M EDTA, 5% (w/v) SDS, 0.5 mg/ml proteinase K.

### Plasmids

An expression plasmid (pET 24 d(+) Novagen) was engineered to produce I-DmoI/I-CreI hybrid meganuclease with or without an histidin tag. Briefly, a fragment containing the ORF sequence and flanked by NcoI and EagI or XhoI restriction sites was prepared using the polymerase chain reaction (PCR) and specific oligonucleotides. Firstly, the half I-DmoI and I-CreI sequences were prepared by PCR then I-DmoI/I-CreI hybrid sequence was completed by another PCR.

### Expression and Purification of I-DmoI/I-CreI Hybrid Meganuclease

Escherichia coli strain BL21(DE3) RIL was used for the purification of I-DmoI/I-CreI Clones transformed with the I-DmoI/I-CreI plasmid (pET 24 d(+) Novagen) were grown in 250 ml of Luria Broth containing 30 mg/ml kanamycin at 37° C. with shaking.

When the culture reached an A₆₀₀ of 0.8-1.2, expression was induced by adding IPTG to a final concentration of 1 mM, and after 5 h to 15 h at 25° C., the cells were harvested by centrifugation.

The following procedures were performed at 4° C. unless stated otherwise. The harvested cells were resuspended in 25 ml of ice-cold sonication solution, and then sonicated for 5 minutes. The lysate was centrifuged at 105 000 g for 30 min. The supernatant recovered, and then subjected to a second ultracentrifugation at 105 000 g for 30 min. This supernatant, which contained 90% of the protein, was called the ‘soluble’ fraction.

The soluble fraction was applied to a ml Hi-Trap chelating column (Amersham, Uppsala, Sweden) load with cobalt at a flow rate of 2.5 ml/min (Amersham—Pharmacia FPLC Akta purifier 10). After washing the column with 25 ml of solution A, bound proteins were eluted with a 0-0.25 M linear imidazole gradient made up in solution A following by an elution step at 0.5M imidazole-0.5M NaCl. Fractions were collected, and the amounts of protein and I-DmoI/I-CreI activity (see below) were determined. The column fractions were also analyzed by SDS-PAGE. The fractions containing most of the I-DmoI/I-CreI activity were pooled and concentrated using a 10 kDa cut-off centriprep Amicon system. This concentrated fraction was purified was applied to a Superdex75 PG Hi-Load 26-60 column (Amersham, Uppsala, Sweden) at a flow rate of 1 ml/min (Amersham—Pharmacia FPLC Akta purifier 10) of solution A. Fractions were collected, and the amounts of protein and I-DmoI/I-CreI activity (see below) were determined. The column fractions were also analyzed by SDS-PAGE. The fractions containing most of the I-DmoI/I-CreI activity were pooled and concentrated using a 10 kDa cut-off centriprep Amicon system. Then dialysed against storage solution over-night, and stored in aliquots in liquid nitrogen.

### SDS-Polyacrylamide Gel Electrophoresis

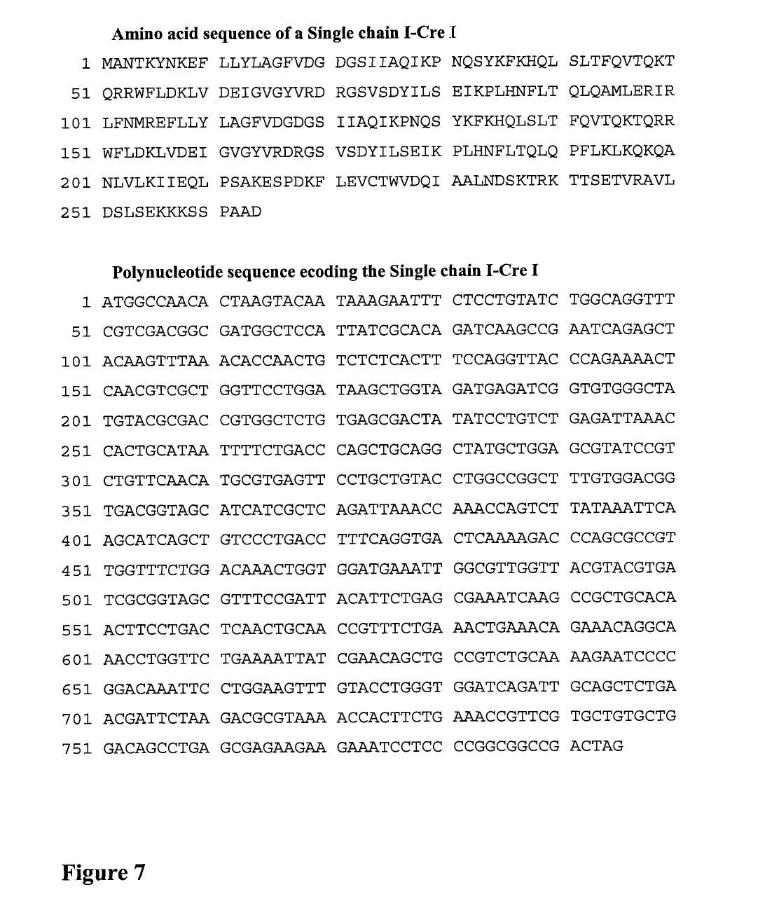

SDS-polyacrylamide gel electrophoresis (SDS-PAGE) was performed as described by Laemmli using 15% acrylamide gels. Gels were stained with coomassie brilliant blue. FIG. 8A shows that the hybrid meganuclease I-DmoI/I-CreI is well expressed and that this hybrid is obtained in the supernatant and therefore is soluble.

### Size of I-DmoI/I-CreI Hybrid Meganuclease

The molecular weight of I-DmoI/I-CreI in solution was estimated by size-exclusion chromatography of the purified protein. The column fractions were analyzed by SDS-PAGE, and the 31.2 kDa band eluted primarily, which corresponded to a molecular mass of 30 kDa. Thus, this analysis indicated that I-DmoI/i-CreI is mainly a monomer.

### Example 2

### Single-Chain I-Cre I Meganuclease

Single Chain I-Cre I Meganuclease Design

I-CreI is a single LAGLIDADG protein domain that dimerizes (FIG. 2). For the domain swapping strategy, we planned to connect an I-CreI domain to any other LAGLIDADG domain. Engineering a single chain I-CreI protein, by placing a connecting link between the two monomers, was complementary to the example I. This did not provide a protein with novel DNA binding specificity. Instead, the resulting artificial protein illustrated that natural single LAGLIDADG proteins can be effectively thought of as the two halves of a larger double LAGLIDADG protein. If a fusion of the two domains was functional, swapping the domains from different single LAGLIDADG, between such proteins or with domains from double LAGLIDADG proteins, should be straightforward.

Each I-CreI domain comprises a C-terminal sub-domain made of three α-helices, which may be present in the C-terminal domain of double LAGLIDADG proteins, but cannot be part of the N-terminal domain. The N-terminal domain of these proteins is indeed shorter, as the loop or linker connecting the two domains stands in place of the three α-helices. The three helices terminate at opposite sides of the dimer structure, far away from the N-terminal helices of the dodecapeptide motifs. The length of a flexible linker connecting the C-terminal residue of one domain to the N-terminal residue of the other domain (end-to-end fusion) would be considerable. Besides, engineering such linker would be difficult due to the necessity to go across a large part of the protein surface. Therefore, it is uncertain that proper domain packing be obtained.

The structural superimposition of I-DmoI and I-CreI discussed in the previous example, allowed to design a simple linker solution. The loop region from I-DmoI itself, residues 96 to 103 (sequence ERIRLFNM), may replace the C-terminal fragment of one I-CreI domain and lead to the N-terminal region of the second domain (as it does in the I-DmoI/I-CreI hybrid protein, except that the residues at the beginning of the I-CreI α-helix need not and should not be replaced). On both side of the loop, residues from I-DmoI and I-CreI can be well aligned and superimposed. These residues are:

| | | | | Atoms |
|---|---|---|---|---|
| Source residues | Sequence | Target residues | Sequence | superimposed |
| I-DmoI, 93-95 | NML | I-CreI, 93-95 | PFL | N, Cα, C, O |
| I-DmoI, | REQ | I-CreI, 207-209 | KEF | N, Cα, C, O |
| 104-106 | | | | |

The link between the I-CreI domains should thus be chosen to replace the C-terminal residues of the first domain and the N-terminal residues of the second, respectively from either Pro93, Phe94 or Leu95 and up to Lys207, Glu208 or Phe209. Actually, amino acids at two of the superimposed positions are identical (Leu95 in both proteins and Glu105 from I-DmoI with Glu208 from I-CreI; underlined residues), and at a third position they are sufficiently similar to be equivalent (Arg104 and Lys207 from I-DmoI and I-CreI, respectively; underlined residues).

The lysine 98 of the first domain I-CreI has been removed despite of the disclosure of Jurica et al (1998, Mol. Cell., 2, 469-476) saying that Lysine 98 in I-CreI which is a well-conserved residue and is close to the active (cleavage) site could have a functional role.

Proline 93 is not an optimal residue for the α-helical conformation of the main chain at that residue position. The corresponding asparagine in I-DmoI is only slightly better; particular hydrogen bonding properties of the amino acid establish a pronounced tendency to promote a N-terminal break in α-helices. At that position, an alanine residue is eventually preferred (glutamic acid would be another suitable amino acid, which has good intrinsic propensity to adopt a α-helical conformation and could form a stabilizing salt-bridge with Arg97 in the following linker region).

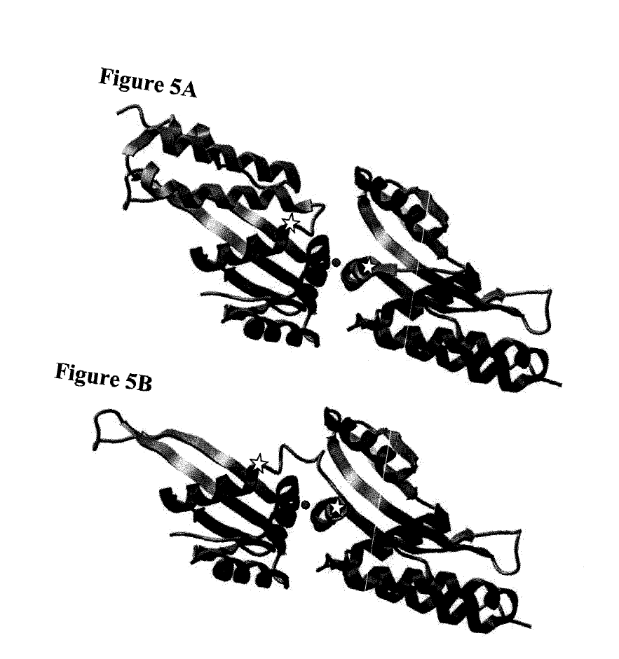

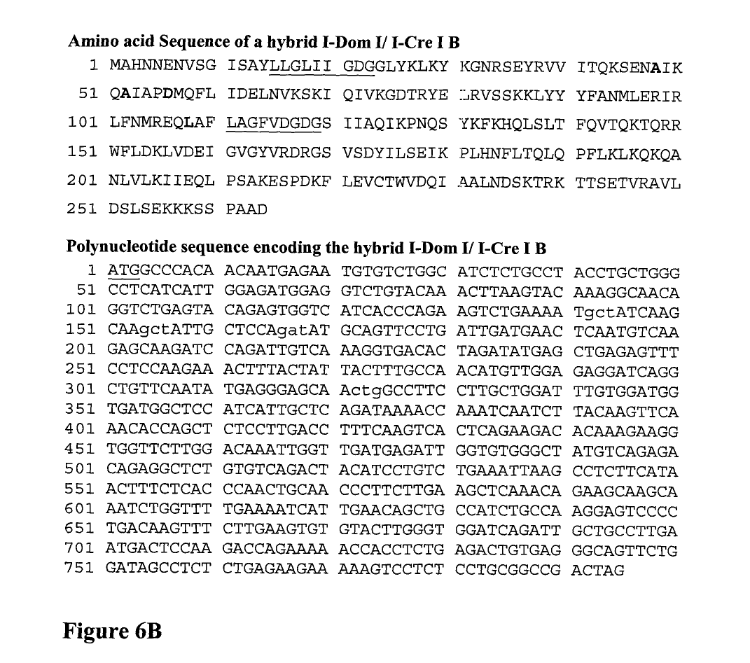

A single chain version of I-CreI was thus engineered so as to introduce the I-DmoI bridge, starting with Met94 and up to Glu105, between a shortened version of the natural I-CreI domain (truncation after the Pro93Ala mutation) and a nearly complete copy of that domain (truncation before Phe209) (FIG. 5 and FIG. 7 for the amino acid (SEQ ID N^{o} 6) and polynucleotide sequences (SEQ ID N^{o} 5)). Met94 replaced a bulkier phenylalanine amino acid that is buried into the I-CreI protein dimer. Both amino acids appear to fit equally well, and could be tried alternatively at that sequence position. Another non-polar amino acid, isoleucine at position 98 (I-DmoI numeration) packs into the original dimer structure without creating any atomic overlaps. Residue 101, an aromatic phenylalanine, is equally fine but could be replaced by another aromatic amino acid, tyrosine, which could then form a stabilizing hydrogen bond to the main chain carbonyl group of residue 94. The following sequences are thus alternative solutions to provide a linker region between two I-CreI domains:

| I-CreI | Linker | I-CreI |
|---|---|---|
| (N-term) . . . TQLQ | AMLERIRLFNMR | EFLL . . . (C- |
| | (SEQ ID N^{o} 8) | term) |
| (N-term) . . . TQLQ | AFLERIRLFNMR | EFLL . . . (C- |
| | (SEQ ID N^{o} 9) | term) |
| (N-term) . . . TQLQ | AMLERIRLYNMR | EFLL . . . (C- |
| | (SEQ ID N^{o} 10) | term) |
| (N-term) . . . TQLQ | AFLERIRLYNMR | EFLL . . . (C- |
| | (SEQ ID N^{o} 11) | term) |

Single Chain I-Cre I Meganuclease Production

### Solutions

Sonication solution: 25 mM HEPES (pH 7.5), 5% (v/v) glycerol, 0.1% (v/v) anti-proteases solution;

Solution A: 25 mM HEPES (pH 7.5), 5% (v/v) glycerol,

Storage solution: 25 mM HEPES (pH 7.5), 20% (v/v) glycerol

Standard reaction solution: 12.5 mM HEPES (pH 7.5), 2.5% (v/v) glycerol, 5-10 mM MgCl₂;

Standard stop solution (10×): 0.1 M Tris-HCl pH 7.5, 0.25 M EDTA, 5% (w/v) SDS, 0.5 mg/ml proteinase K.

### Plasmids

An expression plasmid (pET 24 d(+) Novagen) was engineered to produce Single chain I-CreI (Sc I-CreI) with or without an histidine tag. Briefly, a fragment containing the ORF sequence and flanked by NcoI and EagI or XhoI restriction sites was prepared using the polymerase chain reaction (PCR) and specific oligonucleotides. Firstly, the two half modified I-CreI sequences were prepared by PCR then the single chain I-CreI hybrid sequence was completed by another PCR.

Expression and Purification of Sc I-CreI (Sc=single chain)

Escherichia coli strain BL21(DE3) RIL was used for the production and the purification of Sc I-CreI polypeptide. Clones transformed with the Sc I-CreI plasmid (pET 24 d(+) Novagen) were grown in 250 ml of Luria Broth containing 30 mg/ml kanamycin at 37° C. with shaking.

When the culture reached an A₆₀₀ of 0.8-1.2, expression was induced by adding IPTG to a final concentration of 1 mM, and after 5 h to 15 h at 20° C., the cells were harvested by centrifugation.

The following procedures were performed at 4° C. unless stated otherwise. The harvested cells were resuspended in 25 ml of ice-cold sonication solution, and then sonicated for 5 minutes. The lysate was centrifuged at 105 000 g for 30 min. The supernatant recovered, and then subjected to a second ultracentrifugation at 105 000 g for 30 min. This supernatant, which contained 90% of the protein, was called the ‘soluble’ fraction.

The soluble fraction was applied to a 5 ml Hi-Trap chelating column (Amersham, Uppsala, Sweden) load with cobalt at a flow rate of 2.5 ml/min (Amersham—Pharmacia FPLC Akta purifier 10). After washing the column with 25 ml of solution A, bound proteins were eluted with a 0-0.25 M linear imidazole gradient made up in solution A following by an elution step at 0.5M imidazole-0.5M NaCl. Fractions were collected, and the amounts of protein and Sc I-CreI activity (see below) were determined The column fractions were also analyzed by SDS-PAGE. The fractions containing most of the Sc I-CreI activity were pooled and concentrated using a 10 kDa cut-off centriprep Amicon system. This concentrated fraction was purified was applied to a Superdex75 PG Hi-Load 26-60 column (Amersham, Uppsala, Sweden) at a flow rate of 1 ml/min (Amersham—Pharmacia FPLC Akta purifier 10) of solution A. Fractions were collected, and the amounts of protein and Sc I-CreI activity (see below) were determined. The column fractions were also analyzed by SDS-PAGE. The fractions containing most of the Sc I-CreI activity were pooled and concentrated using a 10 kDa cut-off centriprep Amicon system. Then dialysed against storage solution over-night, and stored in aliquots in liquid nitrogen.

### SDS-Polyacrylamide Gel Electrophoresis

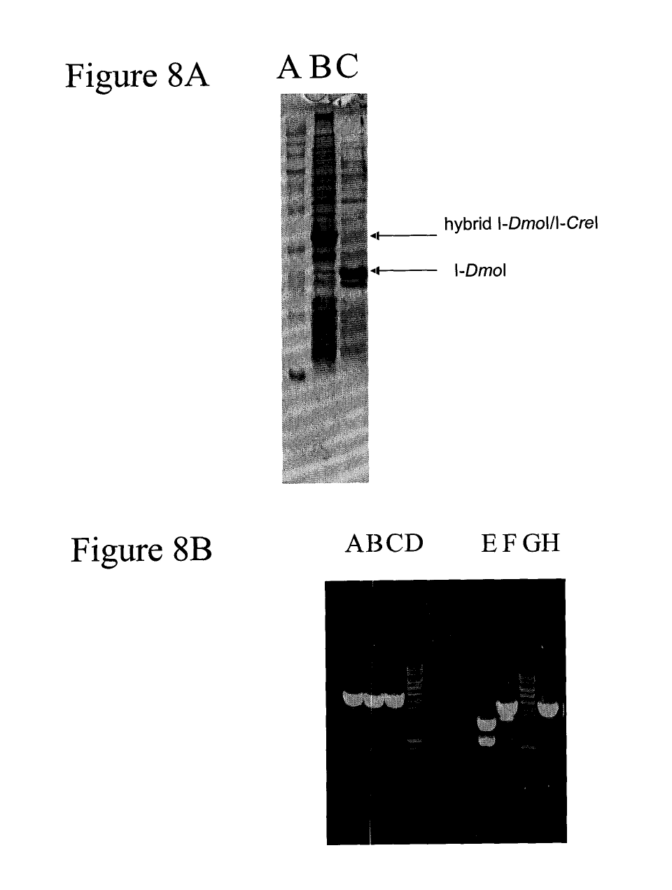

SDS-polyacrylamide gel electrophoresis (SDS-PAGE) was performed as described by Laemmli using 15% acrylamide gels. Gels were stained with coomassie brilliant blue. (FIG. 9A)

### Size of Sc I-CreI Meganuclease

The molecular weight of Sc I-CreI in solution was estimated by size-exclusion chromatography of the purified protein. The results of the sizing column are summarized in FIG. 2A. The column fractions were analyzed by SDS-PAGE, and the 31.4 kDa band eluted primarily, which corresponded to a molecular mass of 30 kDa. Thus, this analysis indicated that Sc I-CreI is mainly a monomer.

### Example 3

### Cleavage Assay

In Vitro Cleavage Assay

Endonuclease Activity Assays of Hybrid I-Dmo I/I-Cre I Meganuclease

Plasmid pGEMtarget (3.9 kb), was constructed by TA or restriction enzyme cloning of the target product, obtained by PCR or single strain hybridation. The target products comprise the following recognition and cleavage sites:

| | | | |
|---|---|---|---|
| wild type | CAAAACGTCGT | GAGACAGTTTGGTCCA | SEQ ID N^{o} 12 |
| I-Cre I | | | |
| wild type | CCTTGCCGGGT | AAGTTCCGGCGCGCAT | SEQ ID N^{o} 13 |
| I-Dmo I | | | |
| L_{(I-Dmo} | CCTTGCCGGGT | GAGACAGTTTGGTCCA | SEQ ID N^{o} 14 |
| _{I)}/R_{(I-} | | | |
| _{Cre I)} | | | |
| target | | | |
| L_{(I-cre} | CAAAACGTCGT | AAGTTCCGGCGCGCAT | SEQ ID N^{o} 15 |
| _{I)}/R_{(I-} | | | |
| _{Dmo I)} | | | |
| target | | | |

This plasmid was used for the cleavage assays. It was isolated with the Quiagen Maxipreps DNA Purification System (Qiagen). For most experiments, it was linearized with XmnI prior to the assay. Standard I-DmoI/I-CreI assays were performed at 37° C. in the standard reaction solution (solution A 0.5×, add MgCl₂ 5 to 10 mM), stopped with 0.1 vol of 10× standard stop solution, and the products separated by electrophoresis in 1% agarose/ethidium bromide gels at room temperature. The fluorescence was captured with camera using a transilluminator.

One unit of endonuclease activity (U) was defined as the amount of I-DmoI/I-CreI necessary to cleave 200 ng of target DNA in 60 min at 37° C. in the same assay condition than I-CreI wild-type. Activity assays were also performed at 65° C. as I-DmoI optimal temperature for DNA cleavage.

The I-DmoI/I-CreI hybrid meganuclease specifically cleaves the target I-CreI/I-DmoI and does not cleave the wild type targets of I-Dmo I, I-Cre I, and I-Sce I meganucleases and the target I-DmoI/I-Cre I. These results are shown in FIG. 8B. Thus, the I-DmoI-CreI hybrid meganuclease shows a high specificity for its new target sequence. The I-DmoI/I-CreI hybrid meganuclease cleaves the target I-CreI/I-DmoI at 37 and 65° C., but the cleavage is more rapid at 65° C. The new specificity of the I-DmoI/I-CreI hybrid meganuclease for the target I-CreI/I-DmoI allows the determination of the relative orientation of the wild type meganuclease I-Dmo I and its recognition and cleavage site. Indeed, the N-terminal domain of I-Dmo I meganuclease recognizes the second half domain of the target I-CreI/I-DmoI. Therefore, the N-terminal domain of I-Dmo I meganuclease recognize the right half site (R) and its C-terminal domain the left one (L).

Endonuclease Activity Assays of Single Chain I-Cre I Meganuclease

Plasmid pGEMtarget (3.9 kb), was constructed by TA or restriction enzyme cloning of the target product, obtained by PCR or single strain hybridation. The target product comprises the recognition and cleavage site

| | | | |
|---|---|---|---|
| wild type | CAAAACGTCGT | GAGACAGTTTGGTCCA | SEQ ID N^{o} 12 |
| I-Cre I | | | |
| wild type | TAGGGAT | AACAGGGTAAT | SEO ID N^{o} 16 |
| I-Sce I | | | |

This plasmid was used for the cleavage assays. It was isolated with the Quiagen Maxipreps DNA Purification System (Qiagen). For most experiments, it was linearized with XmnI prior to the assay. Standard Sc I-CreI assays were performed at 37° C. in the standard reaction solution (solution A 0.5×, add MgCl₂ 5 to 10 mM), stopped with 0.1 vol of 10× standard stop solution, and the products separated by electrophoresis in 1% agarose/ethidium bromide gels at room temperature. The fluorescence was captured with camera using a transilluminator.

One unit of endonuclease activity (U) was defined as the amount of Sc I-CreI necessary to cleave 200 ng of target DNA in 60 min at 37° C. in the same assay condition than I-CreI wild-type. Activity assays were also performed at 65° C. to compare with the wild type I-CreI about effect of temperature on DNA cleavage.

The single chain meganuclease kept its specificty as this meganuclease did not cleave the I-Sce I target site and cleaved the wild type I-Cre I target site. These results are shown in FIG. 9B.

In Vivo Cleavage Assay in Yeast

Endonuclease Activity Assays of Single Chain I-Cre I Meganuclease

To test the meganucleases, we developed an in vivo assay the yeast Saccharomyces cerevisiae. This organism has the advantage to recombine naturally its DNA via homologous recombination.

This test was based on the reparation of a colorimetric marker induced by site-specific double-stand break by the meganuclease of interest.

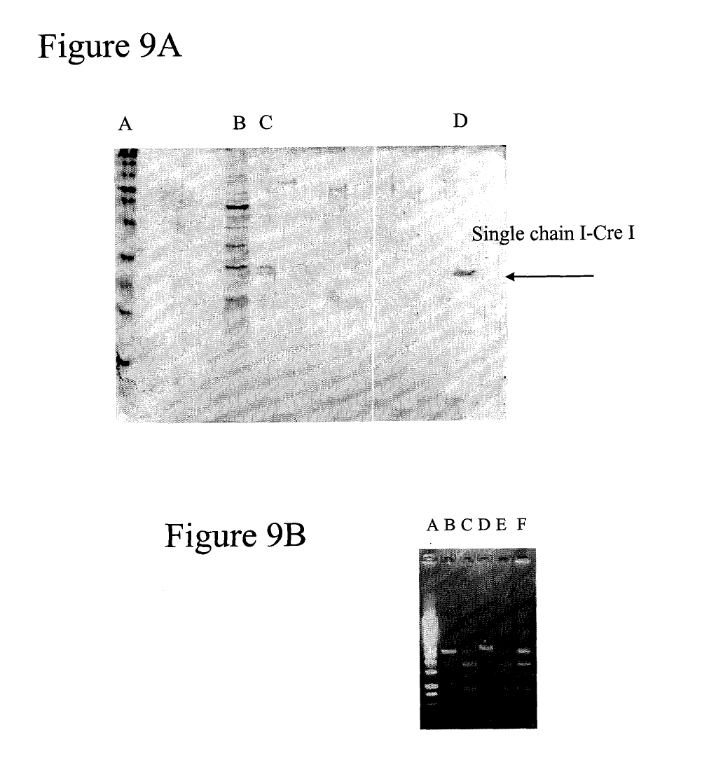

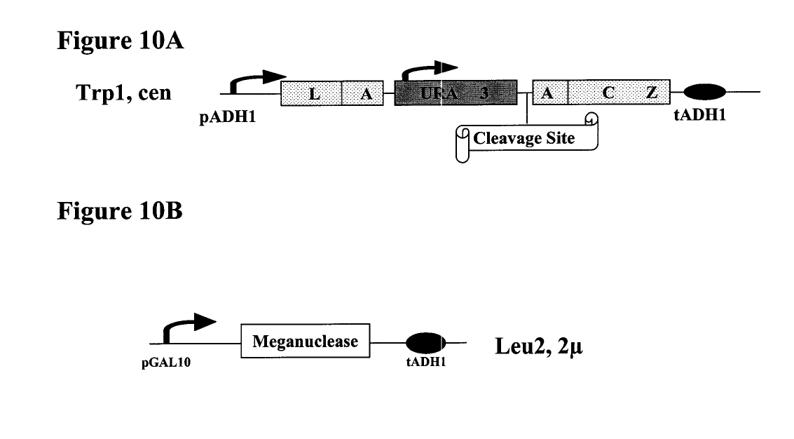

The target consisted of a modified beta-galactosidase gene with a 900 pb internal duplication separated by the Ura3 selectable marker and a cleavage site for the meganuclease to be studied (the resulting construct has been called LACURAZ) (FIG. 10A).

The meganuclease was expressed under the control of a galactose-inducible promoter from a nuclear expression plasmid which carried the Leu2 selectable marker allowing transformed yeast to grow on a media that do not contain any Leucine, and a 2μ, origin of replication (FIG. 10B). The expression of the reporter gene (LACURAZ) was controlled by a constitutive promoter and was carried by another shuttle vector with the Trp1 selectable marker allowing transformed yeast to grow on a media that did not contain any tryptophane and an ARS-CEN origin of replication (FIG. 10A).

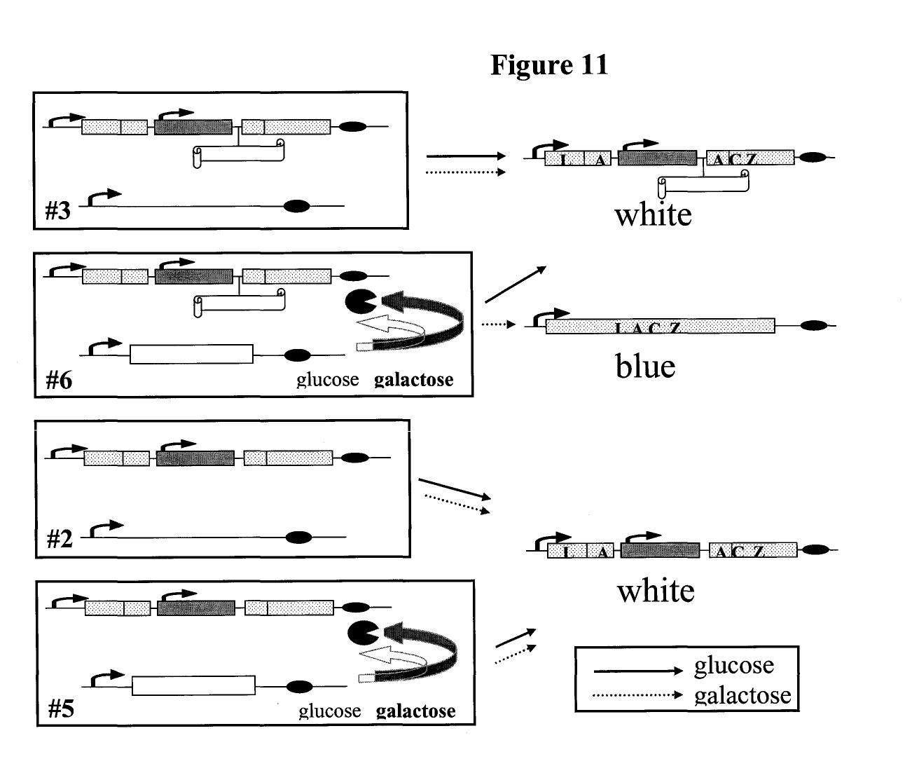

The two constructs were used to transform simultaneously an appropriate yeast strain. If the meganuclease is expressed (or overexpressed after induction on a galactose media) and recognizes its cleavage site on the reporter construct, it can promote double-stand break and site-specific recombination between the target sequences by a Single-Strand Annealing mechanism. The resulting reporter gene should then express fully active beta-galactosidase. We also prepared all the control experiments with plasmids that do not express any meganuclease gene and reporter construct with no cleavage site (all the possible event are described in FIG. 11).

The assay is performed as follow:

The yeast was co-transfected with the expression plasmid and the reporter plasmid. The co-transformants were selected, and the presence of the two plasmids was achieved by selection on a synthetic media containing no Leucine nor Tryptophane (Table A). In addition, two sets of media were used allowing the overexpression of the meganuclease gene or not (i.e. on selective media with different carbon source: galactose to induce the expression of the meganuclease or glucose). When colonies were big enough, an overlay assay revealed the presence or absence of the beta-galactosidase activity.

Theoretically, beta-galactosidase activity should only be detected when a meganuclease and its own recognition site are present in the same yeast cell (except the natural background of autonomous recombination of the reporter construct) (see FIG. 10).

Following this schema, we subcloned the I-CreI Single-chain gene into our galactose inducible plasmid as well as the I-CreI recognition site in our reporter construct and co-transfected both plasmids into our yeast strain. (As a control we use an I-CreI gene on the same reporter construct).

TABLE A
Number of tranformation per assay

| Reporter Construct/ | Empty | LACURAZ control | LACURAZ + |
|---|---|---|---|
| Expression Vector | plasmid | with no cleavage site | cleavage site |
| Empty plamid | # 1 | # 2 | # 3 |
| Meganuclease's gene | # 4 | # 5 | # 6 |

Each transformation is plated on glucose and galactose media.

Methods:

### Yeast Cell Transformation

1. Inoculate 2-5 mls of liquid YPGlu or 10 ml minimum media and incubate with shaking overnight at 30° C.

2. Count on culture and inoculate 50 ml of warm YPGlu to a cell density of 5×10⁶ cells/ml culture.

3. Incubate the culture at 30° C. on a shaker at 200 rpm until its equivalent to 2×10⁷ cells/ml. This takes 3 to 5 hours. This culture gives sufficient cells for 10 transformations.

4. Harvest the culture in a sterile 50 ml centrifuge tube at 3000 g (5000 rpm) for 5 min.

5. Pour off the medium, resuspend the cells in 25 ml of sterile water and centrifuge again.

6. Pour off the water, resuspend the cells in 1 ml 100 mM LiAc (lithium acetate) and transfer the suspension to a 1.5 ml microfuge tube.

7. Pellet the cells at top speed for 15 sec and remove the LiAc with a micropipette.

8. Resuspend the cells to a final volume of 500 μl (2×109 cells/ml) (about 400 μl of 100 mM LiAc).

9. Boil a 1 ml sample of Salmon Sperm-DNA for 5 min. and quickly chill in ice water.

10. Vortex the cell suspension and pipette 50 μl samples into labelled microfuge tubes. Pellet the cells and remove the LiAc with a micropipette.

11. The basic “transformation mix” consists of:

240 μl PEG (50% w/v)

36 μl 1.0 M. LiAc

50 μl SS-DNA (2.0 mg/ml)

X μl Plasmid DNA (0.1-10 μg)

34-X μl Sterile ddH2O

360 μl TOTAL

Carefully add these ingredients in the order listed.

(One can also premix the ingredients except for the plasmid DNA then add 355 μl of “transformation mix” on top of the cell pellet. Then add the 5 μl of plasmid DNA and mix. Take care to deliver the correct volume as the “transformation mix” is viscous).

12. Vortex each tube vigorously until the cell pellet has been completely mixed.

13. Incubate at 30° C. for 30 min.

14. Heat shock in a water bath at 42° C. for 30 min

15. Microfuge at 6-8000 rpm for 15 sec and remove the transformation mix with a micropipette.

16. Pipette 1 ml of sterile YPGlu and let the cells at 30° C. for 1 to 2 hours. This allows you to have the same number of transformants growing on you glucose and galactose plates.

17. Microfuge at 6-8000 rpm for 15 sec and remove the supernatant.

18. Pipette 200 μl ml of sterile YPGlu 50% into each tube and resuspend the pellet by pipetting it up and down gently.

17. Plate 100 μl of the transformation onto selective plates.

18. Incubate the plates for 2-4 days to recover transformants.

### X-Gal Agarose Overlay Assay

The following solution is given for 2 plates:

1. Microwave 5 ml of 1% agarose in water

2. Prepare the X-Gal mix with
5 ml of Sodium Phosphate buffer 1M
600 μl of Dimethyl Formamide (DMF)
100 μl of SDS 10%

3. Combine the agarose and the Mix and let cool down to 55° C. with agitation

4. When the above solution is ready, add 20 μl of X-Gal 10% in DMF

5. Using a plastic pipette, cover the surface of each plate of cells with 5 ml of the warm solution. 6. After the agar cools and solidifies, the plates may be incubated at either 25° C., 30° C. or 37° C. The blue color develops in a few hours, depending on the strength of the inducer.

7. Colonies may be picked through the top agar even 5 days later. Just take a sterile Pasteur pipette and poke it through the agar into the desired colony or patch. Then use the tip of the Pasteur pipette to streak a fresh plate and, despite the permeabilization, the cells will grow up.

Results:

1-Control Experiment with I-Cre I:

| Reporter | | | |
|---|---|---|---|
| Construct/ | | LACURAZ | LACURAZ + |
| Expression | | control with | I-CreI |
| Vector | Empty plasmid | no cleavage site | cleavage site |
| Empty | Glucose: white | Glucose: white | Glucose: white |
| plamid | Galactose: white | Galactose: white | Galactose: white |
| I-CreI gene | Glucose: white | Glucose: white | Glucose: light blue |
| | Galactose: white | Galactose: white | Galactose: dark blue |

2-Experiment with Single-Chain I-CreI:

| Reporter | | | |
|---|---|---|---|
| Construct/ | | LACURAZ | LACURAZ + |
| Expression | | control with | I-CreI |
| Vector | Empty plasmid | no cleavage site | cleavage site |
| Empty | Glucose: white | Glucose: white | Glucose: white |
| plamid | Galactose: white | Galactose: white | Galactose: white |
| Single-chain | Glucose: white | Glucose: white | Glucose: light blue |
| I-CreI gene | Galactose: white | Galactose: white | Galactose: dark blue |

These results indicate that the Single-chain I-CreI gene allows the expression of an active meganuclease that behaves like the natural I-CreI molecule by recognizing and cutting its own cleavage site inducing homologous recombination of the reporter sequence. The light blue color is due to a very small expression of the meganuclease which is very stable in the cell. This small amount of protein allows the reporter construct to recombine leading to the production of a detectable β-galactosidase activity. On every “white” plate, some blue colonies appear at an average rate of 10⁻². This background is due to the high spontaneous recombination in yeast.

In Vivo Cleavage Assay in Mammalian Cells

Endonuclease Activity of Single-Chain I-CreI Meganuclease

We also developed an assay in mammalian cells, based on the detection of homologous recombination induced by targeted double-strand break. As in the yeast system described above, we monitor the restoration of a functional LacZ marker resulting from the cleavage activity of the meganuclease of interest.

We first transferred the yeast reporter system in a mammalian expression plasmid. The LACZ repeat, together with the intervening sequence, including an I-CreI cleavage site, was cloned into pcDNA3 (Invitrogene). Thus, any functional LACZ gene resulting from recombination would be under the control of the CMV promoter of pcDNA3, and display also functional termination sequences. We also cloned the I-CreI and single-chain I-CreI open reading frames in pCLS3.1, a home-made expression vector for mammalian cells.

Using the Effectene (Qiagen) transfection kit, 0.5 μg of the reporter plasmid was cotransfected into simian COS cells, with 0.5 μg of pCLS3.1, 0.5 μg of the I-CreI expressing plasmid, or 0.5 μg of the single-chain I-CreI expressing plasmid. The beta-galactosidase activity was monitored 72 hours after transfection by an assay described below. In a control experiment, 0.5 μg of a reporter plasmid without any cleavage site was cotransfected with 0.5 μg of pCLS3.1, 0.5 μg of the I-CreI expressing plasmid, or 0.5 μg of the single-chain I-CreI expressing plasmid.

This assay is based on the detection of tandem repeat recombination, which often occurs by a process referred to as SSA (Single-strand Annealing). Therefore, we also designed an assay based on the detection of recombination between inverted repeats. Recombination between inverted repeats mostly occurs by gene conversion, another kind of recombination event.

A complete LacZ ORF, interrupted by an I-CreI cleavage site (and thus not functional) is introduced into pcDNA3, between the CMV promoter and the termination sequence. A truncated non functional copy of the gene is cloned in an inverted orientation, to provide an homologous donor template for gene conversion. It is not flanked by promoter and terminator sequences. It encompasses 2.5 Kb of the inner part of the LacZ ORF, overlapping the region where the I-CreI cleavage site is found in the other copy.

0.5 μg of the reporter plasmid was co transfected into COS cells, with 0.5 μg of pCLS3.1, 0.5 μg of the I-CreI expressing plasmid, or 0.5 μg of the single-chain I-CreI expressing plasmid. In a control experiment, 0.5 μg of a reporter plasmid without any homologous donor template was cotransfected with 0.5 μg of pCLS3.1, 0.5 μg of the I-CreI expressing plasmid, or 0.5 μg of the single-chain I-CreI expressing plasmid.

The assay is performed as follow:

COS cells were transfected with Effectene transfection reagent accordingly to the supplier (Qiagen) protocol. 72 hours after transfection, cells were rinsed twice with PBS1X and incubated in lysis buffer (Tris-HCl 10 mM pH7.5, NaCl 150 mM, Triton X100 0.1%, BSA 0.1 mg/ml, protease inhibitors). Lysate was centrifuged and the supernatant used for protein concentration determination and β-galactosidase liquid assay. Typically, 30 μl of extract were combined with 3 μl Mg 100× buffer (MgCl₂ 100 mM, (β-mercaptoethanol 35%), 33 μl ONPG 8 mg/ml and 234 μl sodium phosphate 0.1M pH7.5. After incubation at 37° C., the reaction was stopped with 500 μl of 1M Na₂CO₃ and OD was measured at 415 nm. The relative β-galactosidase activity is determined as a function of this OD, normalized by the reaction time, and the total protein quantity.

### Results

1. Tandem Repeat Recombination:

| | Experiment | | | | | | | | | | | |
|---|---|---|---|---|---|---|---|---|---|---|---|---|
| | #1 | #2 | #3 | #4 | #5 | #6 | #7 | #8 | #9 | #10 | #11 | #12 |
| Reporter vector (with I-CreI cleavage | + | + | + | + | + | + | | | | | | |
| site) | | | | | | | | | | | | |
| Reporter vector (without I-CreI | | | | | | | + | + | + | + | + | + |
| cleavage site) | | | | | | | | | | | | |
| PCLS3.1 | + | + | | | | | + | + | | | | |
| PCLS3.1-I-CreI | | | + | + | | | | | + | + | | |
| PCLS3.1-single-chain-I-CreI | | | | | + | + | | | | | + | + |
| Beta-gal activity (units/mg prot) × 2 | 44 | 45 | 111 | 110 | 87 | 83 | 41 | 40 | 35 | 32 | 54 | 53 |
| 10¹¹ | | | | | | | | | | | | |

2. Inverted Repeat Recombination

| | Experiment | | | | | | | | | | | |
|---|---|---|---|---|---|---|---|---|---|---|---|---|
| | #1 | #2 | #3 | #4 | #5 | #6 | #7 | #8 | #9 | #10 | #11 | #12 |
| Reporter vector (with homologous | + | + | + | + | + | + | | | | | | |
| template) | | | | | | | | | | | | |
| Reporter vector (without homologous | | | | | | | + | + | + | + | + | + |
| template) | | | | | | | | | | | | |
| PCLS3.1 | + | + | | | | | + | + | | | | |
| PCLS3.1-I-CreI | | | + | + | | | | | + | + | | |
| PCLS3.1-single-chain-I-CreI | | | | | + | + | | | | | + | + |
| Beta-gal activity (units/mg prot) × 2 10¹¹ | 8 | 9 | 29 | 32 | 24 | 21 | 4 | 4 | 4 | 5 | 4 | 4 |

These results indicates that the single-chain I-CreI stimulates can induce enough cleavage of an I-CreI cleavage site to stimulate homologous recombination between direct repeats as well as between inverted repeats.

With direct repeats, similar levels of induced recombination were observed with either the single-chain I-CreI (#5-6) or I-CreI (#3-4). This level of recombination represents a 2 to 2.5-fold increase compared to the background level of recombination of the reporter plasmid (#1-2). In addition, no such increase in the recombination level was observed with a reporter plasmid lacking an I-CreI cleavage site (#7-12).

With inverted repeats, the single-chain I-CreI (#5-6) and the I-CreI meganuclease (#3-4) induced a similar stimulation of gene conversion; with a 2.5 to 4-fold increase compared with background level (#1-2). As expected from a bona fide homologous recombination event, no stimulation was observed with a reporter plasmid lacking a homologous donor template (#7-12).

### Example 4

### The Making of a Single Chain I-CreI Derived Meganuclease Cleaving the Human XPC Gene

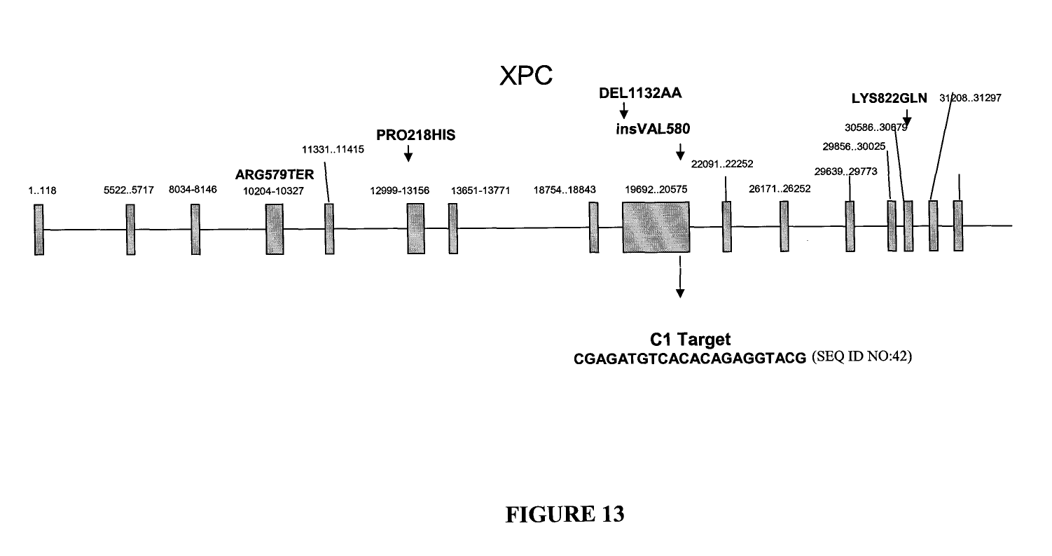

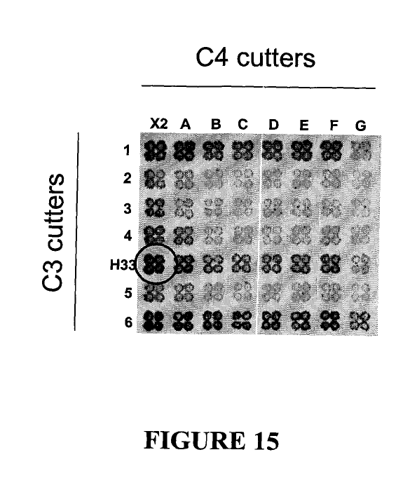

Xeroderma Pigmentosum (XP) is a rare autosomal recessive genetic disease characterized by a hypersensitivity to exposure to ultraviolet (UV) rays and a high predisposition for developing skin cancers. The human XPC gene involved in Xeroderma Pigmentosum was scanned for potential target sequences. A potential 22 bp DNA target that was called C1 (cgagatgtcacacagaggtacg; SEQ ID NO: 42), was localized at the end of the XPC ninth exon (FIG. 13). The engineering of I-CreI derived mutants able to cleave the C1 target has been described previously (Arnould et al., J. Mol. Biol., Epub 10 May 2007; International PCT Application WO 2007/093836 and WO 2007/093918). Briefly, the C1 sequence was divided into two palindromic half-targets called C3 and C4 (FIG. 14). As the C3 target is identical to the 10GAG_P target but with a single difference at position ±6, I-CreI derived mutants able to cleave the 10 GAG_P target were screened against the C3 target. The mutant H33 bearing the single mutation Y33H (substitution at position 33 of a tyrosine by a histidine residue) in comparison to the I-CreI wild-type sequence, was isolated as a strong C3 cutter. The C4 target is a combination of the 10GTA_P and 5TCT_P targets. I-CreI mutants able to cleave the 10GTA_P and E-CreI mutants able to cleave the 5TCT_P target were combined and screened against the C4 DNA target, as described previously (Smith et al., Nucleic Acids Research, 2006, 34, e149; International PCT Applications WO 2007/049095 and WO 2007/057781). The I-CreI mutant called X2 was isolated as a strong C4 cutter. The X2 mutant bears the following mutations in comparison with the I-CreI wild type sequence: Y33H, Q38A, S40Q, Q44K, R68Q, R70S and D75N. The last step consisted in the yeast co-expression of the H33 and X2 I-CreI mutants, as described previously (International PCT Application WO 2006/097854 and Arnould et al., J. Mol. Biol., 2006, 355, 443-458), which resulted in the strong cleavage of the XPC C1 DNA target (FIG. 15).

The X2/H33 XPC heterodimer obtained by coexpression of the two mutants cleaves the C1 target but also the C3 and C4 targets, because of the presence of the two homodimers. To avoid these side effects, a new way for designing a single chain molecule composed of the two I-CreI derived mutants X2 and H33 was conceived. For that purpose, a full length X2 N-terminal mutant was maintained in the single chain design, and several constructs of the type X2-L-H33, where L is a protein linker, were engineered. In this nomenclature, X2 will be referred as the N-terminal mutant and H33 as the C-terminal mutant. Another important issue was the sequence identity of the two mutants in the single chain molecule. In fact, the two I-CreI mutants X2 and H33 have almost the same nucleic sequence, which raises the problem of the stability of such a construct with the risk of a recombination event between the two almost identical sequences. To avoid or at least reduce this possibility, another I-CreI version, called I-CreI CLS, was used to code for the H33 mutant. The nucleic sequence of I-CreI CLS (SEQ ID NO: 40) has been rewritten from the I-CreI wild type sequence (I-CreI wt; SEQ ID NO: 38) using the codon usage and the genetic code degeneracy. It means that I-CreI CLS shares 73% nucleic sequence identity with I-CreI wt and has three single amino acid mutations (T42A, E110W and Q111R), which do not alter I-CreI activity. The Y33H was then introduced in the I-CreI CLS version (SEQ ID NO: 41). Activity of the H33 CLS mutant was checked against the C3 target and was shown to be as strong as for the H33 mutant in the I-CreI wt version.

Using the H33 CLS mutant, 18 single chain versions of the type X2-L-H33 were built, where L is a linker, different for each of the 18 versions. The G19S mutation was also introduced in the C-terminal H33 mutant for two single chain molecules. Activity of these different single chain constructs against the C1 XPC target and its two derivatives C3 and C4 was then monitored in yeast and, for some of them, in CHO cells using an extrachromosomal assay.

1) Material and Methods

a) Introduction of the Y33H Mutation into the I-CreI CLS Version

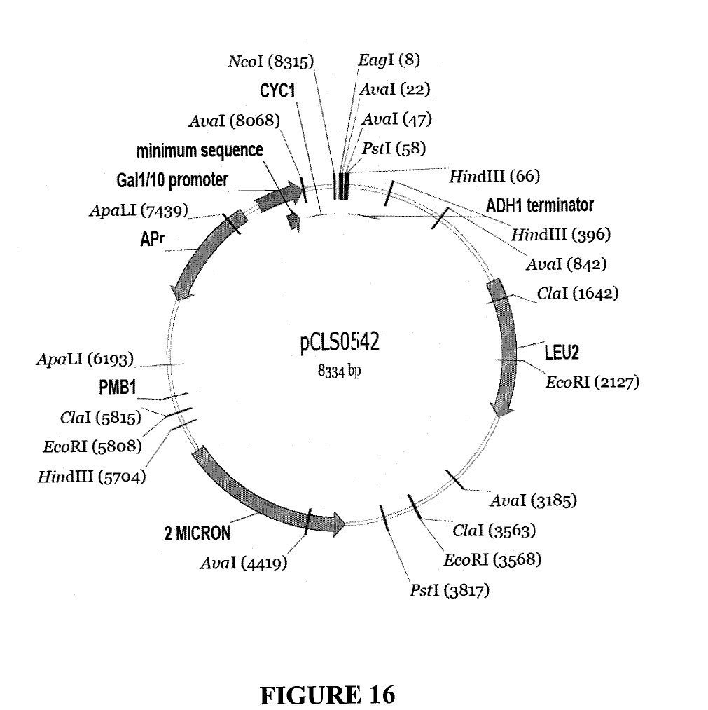

Two overlapping PCR reactions were performed using two sets of primers: Gal10F (5′-gcaactttagtgctgacacatacagg-3′; SEQ ID NO: 60) and H33Revp60 (5′-ctggtgtttgaacttgtgagattgatttggttt-3′; SEQ ID NO: 61) for the first fragment and H33Forp60 (5′-aaaccaaatcaatctcacaagttcaaacaccag-3′; SEQ ID NO: 62) and Gal10R (5′-acaaccttgattggagacttgacc-3′; SEQ ID NO: 63) for the second fragment. Approximately 25 ng of each PCR fragment and 75 ng of vector DNA pCLS0542 (FIG. 16) linearized by digestion with NcoI and EagI were used to transform the yeast Saccharomyces cerevisiae strain FYC2-6A (MATα, trp1 Δ63, leu2Δ1, his3Δ200) using a high efficiency LiAc transformation protocol (Gietz, R. D. and Woods R. A., Methods Enzymol., 2002, 350, 87-96). An intact coding sequence containing the Y33H mutation is generated by in vivo homologous recombination in yeast.

b) Sequencing of Mutants

To recover the mutant expressing plasmids, yeast DNA was extracted using standard protocols and used to transform E. coli. Sequencing of mutant ORF was then performed on the plasmids by MILLEGEN SA.

c) Cloning of the Eighteen XPC Single Chain Molecules

Eighteen independent PCR reactions were performed on the H33 mutant in the I-CreI CLS version. Each PCR reaction uses the same reverse primer CreCterR60 (5′-tagacgagctcctaaggagaggactttttcttctcag-3′; SEQ NO: 64) and a specific forward primer. The eighteen forward primers that were used are:

(SEQ ID NO: 65)

L1EagI:
5′tatcggccggtggcggaggatctggcggcggtggatccggtggtggag
gctccggaggaggtggctctaacaaagagttcctgctgtatcttgctgg
a-3′

(SEQ ID NO: 66)

YPP:
5′tatcggccggtaaatcttccgattccaagggtattgatctgactaatg
ttactctgcctgatacccctacttattccaaagctgcctctgatgctatt
cctccagctaacaaagagttcctgctgtatcttgctggattt-3′

(SEQ ID NO: 67)

AOL:
5′tatcggccggtctggagtatcaggctccttactcttcccctccaggtc
ctccttgttgctccggttcctctggctcctctgctggttgttctaacaaa
gagttcctgctgtatcttgctggattt-3′

(SEQ ID NO: 68)

CXT:
5′-tatcggccggtctgtcctatcattattctaatggtggctcccctact
tctgatggtccagctctgggtggcatttctgatggtggcgctactaacaa
agagttcctgctgtatcttgctggattt-3′,

(SEQ ID NO: 69)

BQY:
5′-tatcggccggtgattcctctgtttctaattccgagcacattgctcct
ctgtctctgccttcctctcctccatctgttggttctaacaaagagttcct
gctgtatcttgctggattt-3′

(SEQ ID NO: 70)

VSG:
5′-tatcggccggtgcttctcagggttgtaaacctctggctctgcctgag
ctgcttactgaggattcttataatactgataacaaagagttcctgctgta
tcttgctggattt-3′

(SEQ ID NO: 71)

BYM:
5′-tatcggccggtaatcctattcctggtctggatgagctgggtgttggc
aactctgatgctgccgctcctggcactaacaaagagttcctgctgtatct
tgctggattt-3′

(SEQ ID NO: 72)

MCJ:
5′tatcggccggtgctcctactgagtgttctccttccgctctgacccagc
ctccatccgcttctggttccctgaacaaagagttcctgctgtatcttgct
ggattt-3′

(SEQ ID NO: 73)

GSmid:
5′-tatcggccggtggaggcggttctggaggcggtggctctggtggaggc
ggttccggtggaggcggatctggtggaggcggttctaacaaagagttcct
gctgtatcttgctggatt-3′

(SEQ ID NO: 74)

GSshort:
5′-tatcggccggtggaggcggttctggaggcggtggctctggtggaggc
ggttccaacaaagagttcctgctgtatcttgctggattt-3′

(SEQ ID NO: 75)

GSxshort:
5′tatcggccggtggaggcggttctggaggcggtggctctaacaaagagt
tcctgctgtatcttgctggattt3′

(SEQ ID NO: 76)

PPR:
5′tatcggccggtcaggttacttctgctgccggtcctgctactgttccat
ctggtaacaaagagttcctgctgtatcttgctggattt-3′

(SEQ ID NO: 77)

SBA1:
5′-tatcggccggtggatctcctctgaagccttctgccccaaagattcct
ataggtggctccaacaaagagttcctgctgtatcttgctggattt-3′

(SEQ ID NO: 78)

SBA2:
5-′tatcggccggtggatctcctctgaagccttctgccccaaagattcct
ataggtggctccccactgaaaccttccgcacctaaaatcccaattggtgg
ctctaacaaagagttcctgctgtatcttgctggattt-3′

(SEQ ID NO: 79)

LP1:
5′-tatcggccggtggatctcctctgtctaaaccaattccaggcggttcc
aacaaagagttcctgctgtatcttgctggattt-3′

(SEQ ID NO: 80)

LP2:
5′tatcggccggtggatctcctctgtctaaaccaattccaggcggttccc
cactgtcaaagccaatccctggcggttctaacaaagagttcctgctgtat
cttgctggattt-3′

(SEQ ID NO: 81)

RM1:
5′-tatcggccggtggatctgataagtataatcaggctctgtctgagcgt
cgcgcctacgttgtcgccaataacctggtttccggtggaggcggttccaa
caaagagttcctgctgtatcttgctggattt-3′

(SEQ ID NO: 82)

RM2:
5′-tatcggccggtggatctgataagtataatcaggctctgtctaaatac
aaccaagcactgtccaagtacaatcaggccctgtctggtggaggcggttc
caacaaagagttcctgctgtatcttgctggattt-3′.

All PCR fragments were purified and digested by EagI and SacI and each PCR fragment was ligated into the yeast expression vector for the X2 mutant also digested with EagI and SacI. After sequencing of the clones, all single chain molecules in the yeast expression vector were obtained.

d) Introduction of the G19S Mutation into the C-Terminal H33 Mutant of the Two Single Chain Molecules X2-L1-H33 and X2-RM2-H33

Two overlapping PCR reactions were performed using two sets of primers: Gal10F (5′-gcaactttagtgctgacacatacagg-3′: SEQ ID NO: 60) and G19SRev (5′ gcaatgatggagccatcagaatccacaaatccagc-3′: SEQ ID NO: 83) for the first fragment and G19SFor60 (5′-gctggatttgtggattctgatggctccatcattgc-3′: SEQ ID NO: 84) and Gal10R (5′-acaaccttgattggagacttgacc-3′: SEQ ID NO: 63) for the second fragment. Approximately 25 ng of each PCR fragment and 75 ng of vector DNA (pCLS0542; FIG. 16) linearized by digestion with NcoI and EagI were used to transform the yeast Saccharomyces cerevisiae strain FYC2-6A (MATα, trp1Δ63, leu2Δ1, his3Δ200) using a high efficiency LiAc transformation protocol (Gietz R. D. and Woods R. A., Methods Enzymol., 2002, 350, 87-96). An intact coding sequence containing the G19S mutation is generated by in vivo homologous recombination in yeast.

e) Introduction of the K7E, E8K and G19S Mutations in the XPC X2-L1-H33 Single

Chain Molecule

First, the G19S mutation was introduced in the X2-L1-H33 molecule. Two overlapping PCR reactions were performed on the single chain molecule cloned in the pCLS0542 yeast expression vector. The first PCR reaction uses the primers: Gal10F (5′-gcaactttagtgctgacacatacagg-3′; SEQ ID NO: 60) and G19SRev60 (5′-gcaatgatggagccatcagaatccacaaatccagc-3′; SEQ ID NO: 83) and the second PCR reaction, the primers G19SFor60 (5′-gctggatttgtggattctgatggctccatcattgc-3; SEQ ID NO: 84) and Gal10R (5′-acaaccttgattggagacttgacc-3′; SEQ ID NO: 63). Approximately 25 ng of each PCR fragment and 75 ng of vector DNA (pCLS0542; FIG. 16) linearized by digestion with NcoI and EagI were used to transform the yeast Saccharomyces cerevisiae strain FYC2-6A (MATα, trp1Δ63, leu2Δ1, his3Δ200) using a high efficiency LiAc transformation protocol (Gietz R. D. and Woods R. A., Methods Enzymol., 2002, 350, 87-96). An intact coding sequence containing the G19S mutation was generated by in vivo homologous recombination in yeast.

In a second step, the K7E and E8K mutations were introduced in the X2-L2-H33(G19S) molecule by performing three overlapping mutations. For the SCX1 molecule, the 3 PCR reactions use three primers set, which are respectively: Gal10F and K7ERev (5% gtacagcaggaactcttcgttatatttggtattgg-3′), K7EFor (5′-aataccaaatataacgaagagttcctgctgtacc-3′; SEQ ID NO: 85) and E8KRevSC (5′-aagatacagcaggaactttttgttagagccacc-3′; SEQ ID NO: 86), E8KForSc (5′-ggtggctctaacaaaaagttcctgctgtatctt-3′; SEQ ID NO: 87) and Gal10R. For the SCX2 molecule, the 3 PCR reactions use three primers set, which are respectively: Gal10F and E8KRev (5′-caggtacagcaggaactttttgttatatttgg-3′; SEQ ID NO: 88), E8KFor (5′-accaaatataacaaaaagttcctgctgtacctgg-3′; SEQ ID NO: 89) and K7ERevSC (5′-aagatacagcaggaactcttcgttagagccacc-3′; SEQ ID NO: 90), K7EForSc (5′-ggtggctctaacgaagagttcctgctgtatctt-3′; SEQ ID NO: 91) and Gal10R. For both constructs, approximately 25 ng of each PCR fragment and 75 ng of vector DNA (pCLS0542; FIG. 16) linearized by digestion with NcoI and EagI were used to transform the yeast Saccharomyces cerevisiae strain FYC2-6A (MATα, trp1Δ63, leu2Δ1, his3Δ200) using a high efficiency LiAc transformation protocol (Gietz R D and Woods R A Transformation of yeast by lithium acetate/single-stranded carrier DNA/polyethylene glycol method. Methods Enzymol. 2002; 350:87-96). An intact coding sequence for the SCX1 or SCX2 constructs was generated by in vivo homologous recombination in yeast.

f) Mating of Meganuclease Expressing Clones and Screening in Yeast

Screening was performed as described previously (International PCT Application WO 2004/067736; Epinat et al., Nucleic Acids Res., 2003, 31, 2952-2962; Chames et al., Nucleic Acids Res., 2005, 33, e178, and Arnould et al., J. Mol. Biol., 2006, 355, 443-458). Mating was performed using a colony gridder (QpixII, Genetix). Mutants were gridded on nylon filters covering YPD plates, using a low gridding density (about 4 spots/cm2). A second gridding process was performed on the same filters to spot a second layer consisting of different reporter-harboring yeast strains for each target. Membranes were placed on solid agar YPD rich medium, and incubated at 30° C. for one night, to allow mating. Next, filters were transferred to synthetic medium, lacking leucine and tryptophan, with galactose (2%) as a carbon source, and incubated for five days at 37° C., to select for diploids carrying the expression and target vectors. After 5 days, filters were placed on solid agarose medium with 0.02% X-Gal in 0.5 M sodium phosphate buffer, pH 7.0, 0.1% SDS, 6% dimethyl formamide (DMF), 7 mM β-mercaptoethanol, 1% agarose, and incubated at 37° C., to monitor (3-galactosidase activity. Results were analyzed by scanning and quantification was performed using appropriate software.

g) Cloning of the XPC Single Chain Constructs into a Mammalian Expression Vector

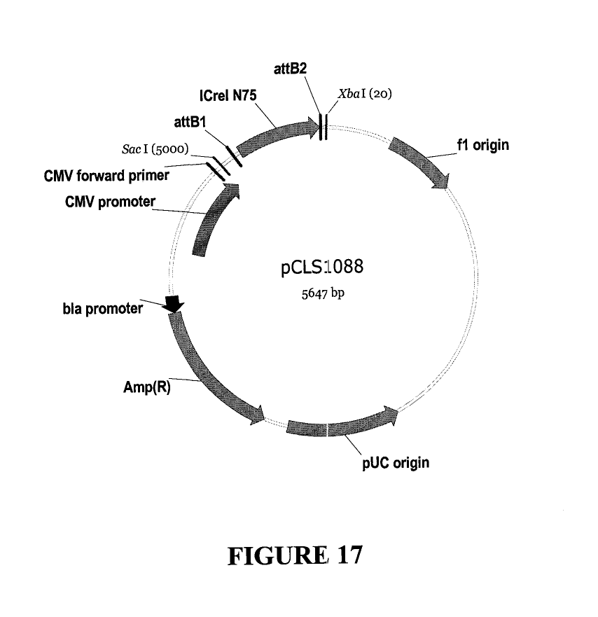

Each mutant ORF was amplified by PCR using the primers CCM2For (5′-aagcagagctctctggctaactagagaacccactgcttactggcttatcgaccatggccaataccaaatataacaaagagttcc-3′: SEQ ID NO: 92) and CCMRev60 (5′-ctgctctagactaaggagaggactttttcttctcag-3′: SEQ ID NO: 93). The PCR fragment was digested by the restriction enzymes SacI and XbaI, and was then ligated into the vector pCLS1088 (FIG. 17) digested also by SacI and XbaI. Resulting clones were verified by sequencing (MILLEGEN).

h) Cloning of the C1, C3 and C4 Targets in a Vector for Extrachromosomal Assay in CHO Cells

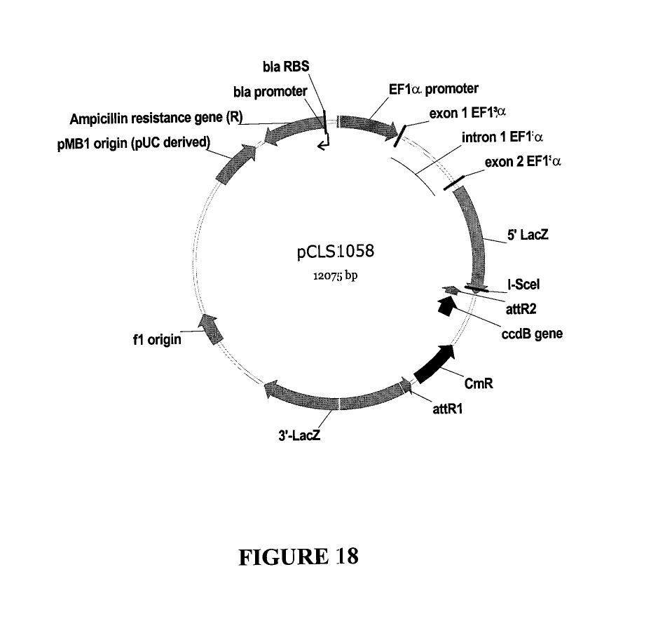

The target of interest was cloned as follow: oligonucleotide corresponding to the target sequence flanked by gateway cloning sequence was ordered from PROLIGO. Double-stranded target DNA, generated by PCR amplification of the single stranded oligonucleotide, was cloned using the Gateway protocol (INVITROGEn) into CHO reporter vector (pCLS1058, FIG. 18).

i) Extrachromosomal Assay in CHO Cells

CHO cells were transfected with Polyfect transfection reagent according to the supplier's (QIAGEN) protocol. 72 hours after transfection, culture medium was removed and 150 μl of lysis/revelation buffer added for β-galactosidase liquid assay (1 liter of buffer contained 100 ml of lysis buffer (Tris-HCl 10 mM pH7.5, NaCl 150 mM, Triton X100 0.1%, BSA 0.1 mg/ml, protease inhibitors), 10 ml of Mg 100× buffer (MgCl₂ 100 mM, β-mercaptoethanol 35%), 110 ml ONPG 8 mg/ml and 780 ml of sodium phosphate 0.1M pH7.5). After incubation at 37° C., OD was measured at 420 nm. The entire process was performed on an automated Velocity11 BioCel platform.

2) Results

a) Cleavage Activity of the 18 XPC Single-Chain Meganucleases Against the Three XPC Targets

Table 1 shows the different linkers that have been used to build the different single chain molecules. For each single chain molecule, the linker begins after the last residue (P163) of the N-terminal X2 mutant and after the linker, the N-terminal H33 mutant begins at residue N6.

TABLE 1
Linkers

| | | | SEQ | |
|---|---|---|---|---|
| | Linker | Size | ID | |
| Number | Name | (aa) | NO: | Primary Sequence |
| 1 | L1 | 22 | 19 | -AA(GGGGS)₄- |
| 2 | YPP | 35 | 20 | - |
| | | | | AAGKSSDSKGIDLTNVTLPDTPTYSKAASDAIPPA- |
| 3 | AOL | 31 | 21 | -AAGLEYPQAPYSSPPGPPCCSGSSGSSAGCS- |
| 4 | CXT | 30 | 22 | -AAGLSYHYSNGGSPTSDGPALGGISDGGAT- |
| 5 | BQY | 27 | 23 | -AAGDSSVSNSEHIAPLSLPSSPPSVGS- |
| 6 | VSG | 25 | 24 | -AAGASQGCKPLALPELLTEDSYNTD- |
| 7 | BYM | 24 | 25 | -AAGNPIPGLDELGVGNSDAAAPGT- |
| 8 | MCJ | 23 | 26 | -AAGAPTECSPSALTQPPSASGSL- |
| 9 | GSmid | 27 | 27 | -AA(GGGGS)₅- |
| 10 | Gsshort | 17 | 28 | -AA(GGGGS)₃- |
| 11 | GSxshort | 12 | 29 | -AA(GGGGS)₂- |
| 12 | PPR | 17 | 30 | -AAGQVTSAAGPATVPSG- |
| 13 | SBA1 | 19 | 31 | -AAGGSPLKPSAPKIPIGGS- |
| 14 | SBA2 | 33 | 32 | -AAGGSPLKPSAPKIPIGGSPLKPSAPKIPIGGS- |
| 15 | LP1 | 15 | 33 | -AAGGSPLSKPIPGGS- |
| 16 | LP2 | 25 | 34 | -AAGGSPLSKPIPGGSPLSKPIPGGS- |
| 17 | RM1 | 31 | 35 | -AAGGSDKYNQALSERRAYVVANNLVSGGGGS- |
| 18 | RM2 | 32 | 18 | -AAGGSDKYNQALSKYNQALSKYNQALSGGGGS- |

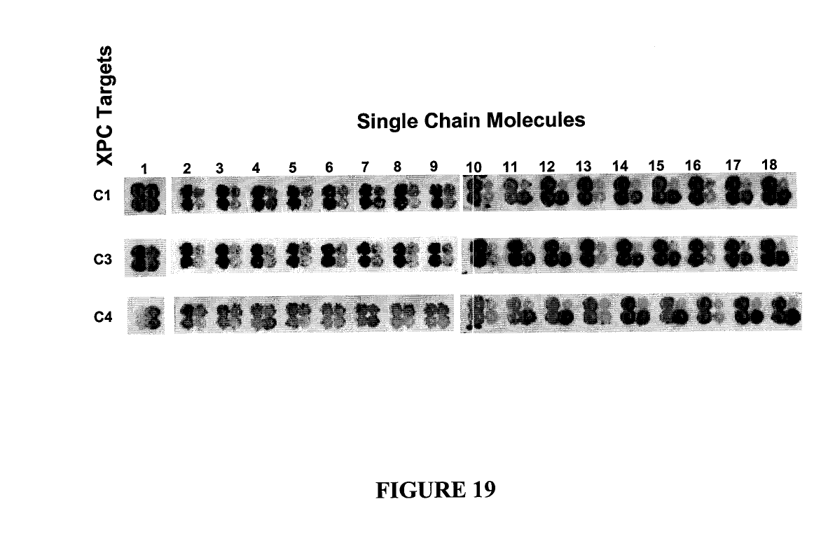

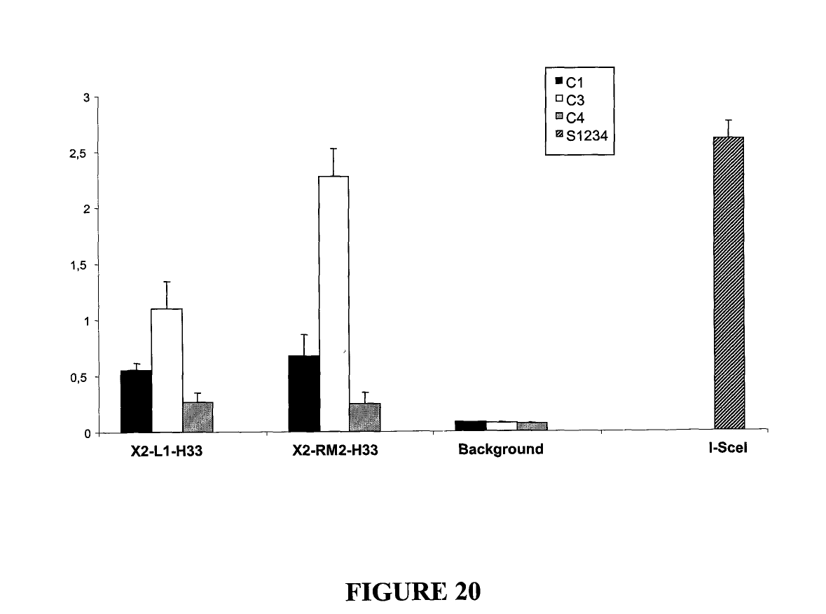

The cleavage activity of the 18 XPC single chain molecules was monitored against the three XPC targets C1, C3 and C4, using the yeast screening assay previously described (International PCT Application WO 2004/067736; Epinat et al., Nucleic Acids Res., 2003, 31, 2952-2962; Chames et al., Nucleic Acids Res., 2005, 33, e178, and Arnould et al., J. Mol. Biol., 2006, 355, 443-458). As shown in FIG. 19, all the single chain constructs cleave the C1 target, but also strongly the C3 target while activity toward the C4 target is also detectable. The two single chain molecules X2-L1-H33 and X2-RM2-H33 were cloned into a mammalian expression vector and their activity toward the three XPC targets was checked in CHO cells using an extrachromosomal assay (FIG. 20). This assay confirmed the yeast cleavage profile (FIG. 20). Furthermore, X2-RM2-H33 is more active toward the C1 target than X2-L1-H33 in CHO cells. C4 cleavage is barely detectable in CHO cells. No cleavage of C3 was observed with X2, and similarly, H33 does not cleave C4 (data not shown). Thus, the strong cleavage of the C3 target with single chain molecules suggests that the linker does not abolish the formation of intermolecular species, resulting from interaction between the dimerization interfaces of the H33 units from two distinct molecules. The formation of pseudo H33 homodimers would then be responsible for C3 cleavage.

b) Effect of the G19S Mutation Alone on the Specificity of Cleavage of the XPC Single-Chain Meganuclease

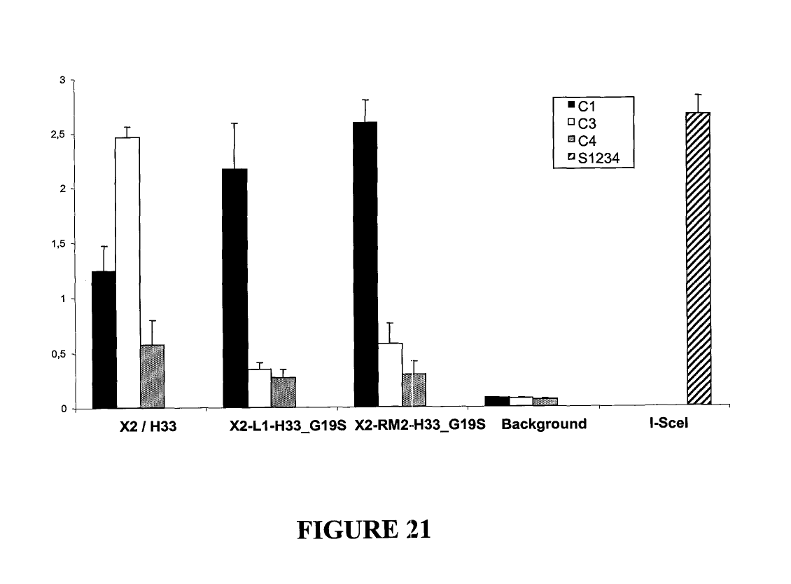

In order to test this hypothesis, the G19S mutation (was introduced in the H33 C-terminal mutant of the two single chain molecules. The G19S mutation (mutation of residue 19 from I-CreI, according to pdb 1G9Y numeration) has been shown before to abolish the formation of functional homodimers while enhancing the activity of the heterodimers displaying a G19S monomer (International PCT Application WO 2008/010093). The two single chain molecules that were obtained (X2-L1-H33_{G19S} and X2-RM2-H33_{G19S}), were then profiled against the three XPC targets using the extrachromosomal assay in CHO cells. FIG. 21 shows that the G19S mutation does not only increases the activity toward the C1 target but also greatly reduces the activity toward the C3 target. The profile cleavage of the X2/H33 heterodimer against the three XPC targets is also shown on FIG. 21, for comparison.

These results confirm the hypothesis of intermolecular species formation, resulting from the interaction of two H33 units. Similarly, interaction between two X2 units probably account for weak cleavage of the C4 target (FIGS. 19 and 20). Thus, although the introduction of a linker between the X2 and H33 monomers might favour intramolecular interactions (resulting in reduced cleavage of C4 for example), it does not abolish intermolecular interactions.

Nevertheless, the engineered XPC single chain molecule X2-RM2-H33_{G19S} cleaves more strongly the C1 XPC target than the X2/H33 heterodimer and has much reduced cleavage activities toward the two palindromic C3 and C4 targets than the same heterodimer. The single chain molecule X2-RM2-H33_{G19S} displays a much better specificity than the X2/H33 heterodimer and has an activity level comparable to that of I-SceI, the gold standard in the field of homologous recombination induced by DNA double strand break.

c) Effect of the Combination of the G19S Mutation with Another Mutation that Impairs the Formation of a Functional Homodimer on the Specificity of Cleavage of the XPC Single-Chain Meganuclease

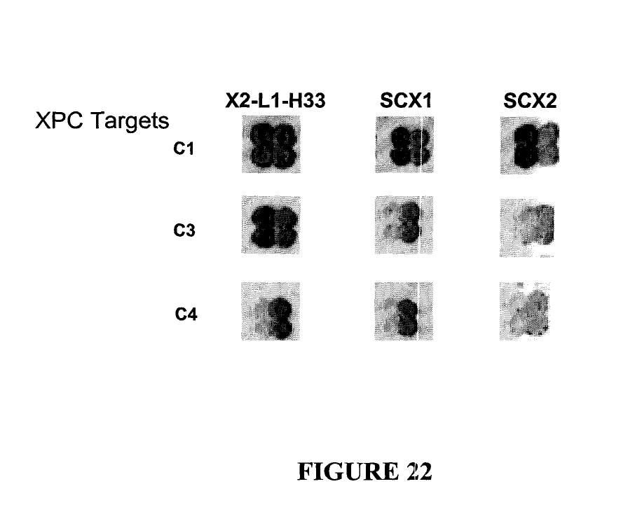

FIG. 22 shows the activity of the three single chain molecules X2-L1-H33, SCX1 and SCX2 against the three XPC targets in a yeast screening assay. The initial single chain molecule has a strong cleavage activity against the C1 and C3 target but introduction of the K7E/E8K and G19S mutations to generate the SCX1 and SCX2 molecules promotes an increased cleavage activity toward the C1 target and a complete abolition of the cleavage activity toward the C3 target. Thus, the mutations K7E/E8K and G19S can be successfully introduced in a single chain molecule to improve its specificity without affecting its cleavage activity toward the DNA target of interest.

### Example 5

### The Making of a Single Chain I-CreI Derived Meganuclease Cleaving the Cricetulus Griseus HPRT Gene

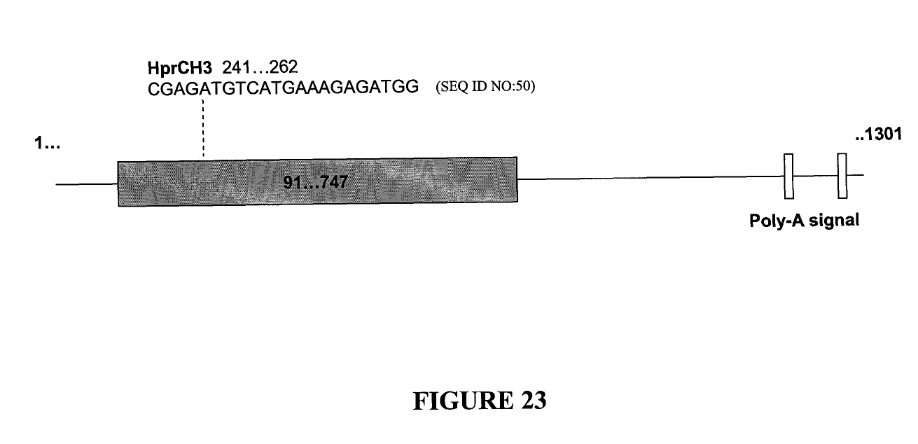

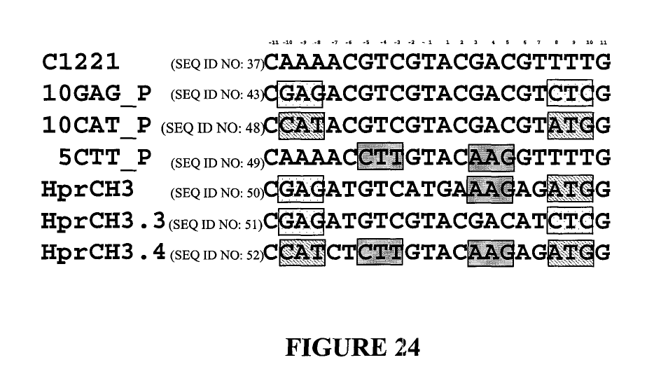

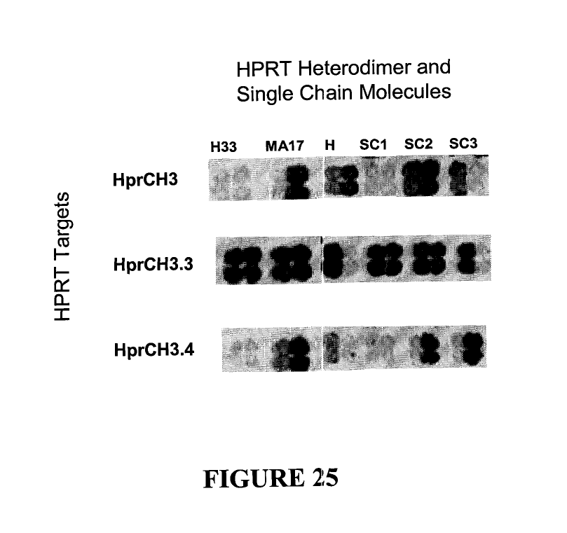

The Hypoxanthine-Guanine Phosphorybosyl Transferase (HPRT) gene from Cricetulus griseus was scanned for a potential target site. A 22 bp sequence called HprCH3 (cgagatgtcatgaaagagatgg: SEQ ID NO: 50) was identified in the gene ORF (FIG. 23). Two derived palindromic targets HprCH3.3 and HprCH3.4 were derived from the HprCH3 target (FIG. 24). As the HprCH3.3 target is identical to the C3 target described above in Example 4, the H33 I-CreI mutant is able to cleave strongly HprCH3.3 (C3). The HprCH3.4 target is a combination of the 10CAT_P and 5CTT_P targets. I-CreI mutants able to cleave the 10CAT_P target were obtained as previously described in Smith et al., Nucleic Acids Research, 2006, 34, e149; International PCT Applications WO 2007/049156 and WO 2007/060495 and I-CreI mutants able to cleave the 5CTT_P target were obtained as previously described in Arnould et al., J. Mol. Biol., 2006; 355, 443-458; International PCT Applications WO 2006/097784 and WO 2006/097853. The mutants were combined as previously described in Smith et al., Nucleic Acids Research, 2006, 34, e149; International PCT Applications WO 2007/049095 and WO 2007/057781 and then screened against the HprCH3.4 DNA target. The I-CreI mutant called MA17 was isolated as a HprCH3.4 cutter. However, it was found to cleave also the HprCH3.3 target, due to a relaxed specificity (see FIG. 25). The MA17 mutant bears the following mutations in comparison with the I-CreI wild type sequence: S32T, Y33H, Q44R, R68Y, R70S, S72T, D75N and I77N. The last step consisted in the yeast co-expression of the H33 and MA17 I-CreI mutants, as described previously (International PCT Application WO 2006/097854 and Arnould et al., J. Mol. Biol., 2006, 355, 443-458), which resulted in the cleavage of the HprCH3 DNA target.

Two HPRT single chain constructs were engineered following the same scheme as in example 4. The two L1 and RM2 linkers (see Table 1 of Example 4) were used, resulting in the production of the MA17-L1-H33 and MA17-RM2-H33 single chain molecules. In a second step, the G19S mutation was introduced in the C-terminal H33 mutant, resulting in a two other single chain meganuclease, MA17-L1-H33_{G19S} and MA17-RM2-H33_{G19S}. The activity of these different constructs was then monitored in yeast and in CHO cells against the HprCH3 target and its two derivatives HprCH3.3 and HprCH3.4 targets.

1) Material and Methods

See example 4

2) Results

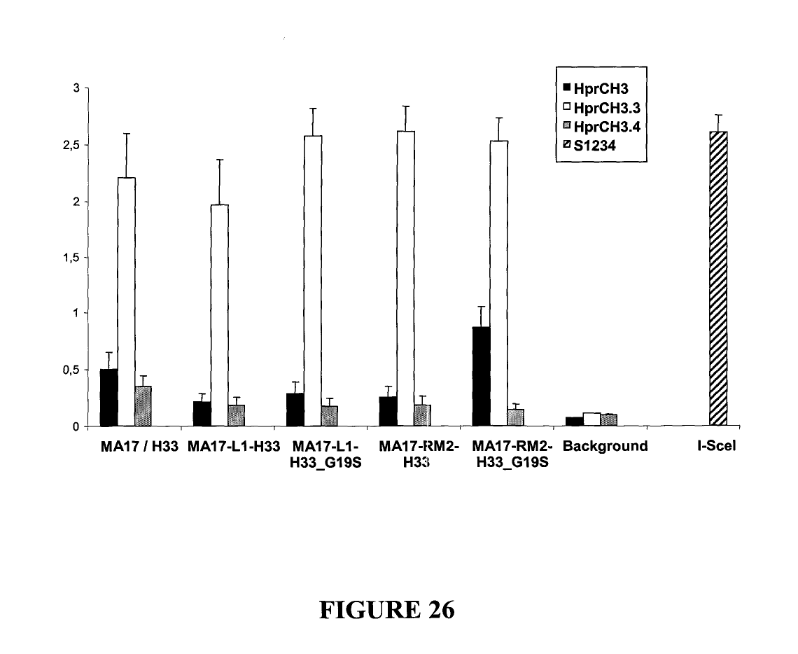

The activity of three single chain molecules (MA17-L1-H33, MA17-L1-H33_{G19S} and MA17-RM2-H33) against the three HPRT targets HprCH3, HprCH3.3 and HprCH3.4 was monitored using the previously described yeast assay (International PCT Application WO 2004/067736; Epinat et al., Nucleic Acids Res., 2003, 31, 2952-2962; Chames et al., Nucleic Acids Res., 2005, 33, e178, and Arnould et al., J. Mol. Biol., 2006, 355, 443-458). As shown in FIG. 25, MA17-L1-H33 does not cleave the HprCH3 target, which is cleaved by the two other single chain molecules. Thus, the RM2 linker seems to be better adapted to the way we have engineered the single chain constructs, as already observed in example 4. These results also confirm that the presence of the G19S mutation enhances the heterodimeric activity. All three single chain molecules cleave very strongly the HprCH3.3 (identical to the C3 target from example 4), but do not cleave the HprCH3.4, in contrast with the MA17/H33 heterodimer. In this case, cleavage of HprCH3.3 (C3) is not necessarily due to the formation of intermolecular species: since the MA17 and H33 mutants both cleave the HprCH3.3 target as homodimers, a MA17/H33 heterodimer or a MA17-RM2-H33 single chain monomer could in principle cleave HprCH3.3. This hypothesis is confirmed by the persistent cleavage of HprCH3.3 by MA17-L1-H33_{G19S} and MA17-RM2-H33_{G19S}. Next, four single chain molecules (MA17-L1-H33, MA17-L1-H33_{G19S}, MA17-RM2-H33 and MA17-RM2-H33_{G19S}) were cloned into a mammalian expression vector tested in CHO cells using for cleavage of the three HPRT targets (FIG. 26). The MA17-RM2-H33_{G19S} single chain molecule displayed the strongest activity. Again strong cleavage of HprCH3.3 (C3) was observed, while cleavage of HprCH3.4 was low or absent.

### Example 6

### The Making of a Single Chain I-CreI Derived Meganuclease Cleaving the Human Rag1 Gene

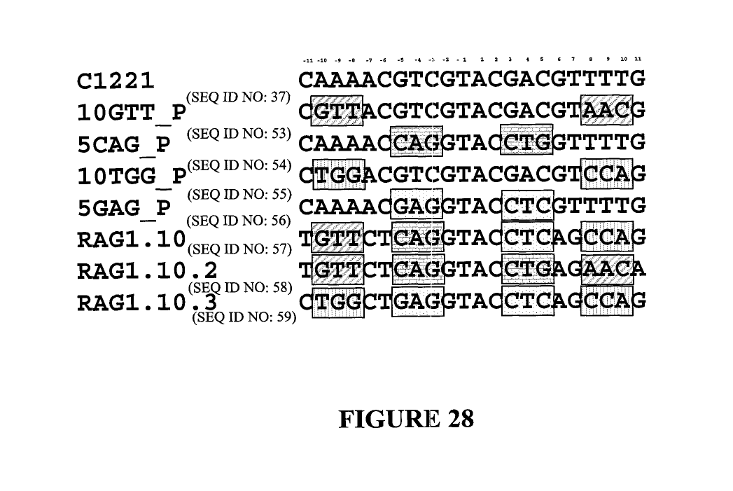

RAG1 is a gene involved in the V(D)J recombination process, which is an essential step in the maturation of immunoglobulins and T lymphocytes receptors (TCRs). Mutations in the RAG1 gene result in defect in lymphocytes T maturation, always associated with a functional defect in lymphocytes B, which leads to a Severe Immune Combined Deficiency (SCID). A 22 bp DNA sequence located at the junction between the intron and the second exon of the human RAG1. called RAG1.10 (SEQ ID NO: 57; FIG. 27) had been identified as a potential cleavable sequence by our meganucleases (Smith et al., Nucleic Acids Research, 2006, 34, e149 International PCT Application WO 2008/010093). The RAG1.10 sequence was derived into two palindromic RAG1.10.2 and RAG1.10.3 sequences (FIG. 28). RAG1.10.2 target is a combination of the 10GTT_P and 5CAG_P targets and RAG1.10.3 target is a combination of the 10TGG_P and 5GAG_P targets. Strong cutters for both RAG1.10.2 and RAG1.10.3 targets were obtained by combining I-CreI mutants able to cleave the 10GTT_P and 5CAG_P targets from one side, and the 10TGG_P and 5GAG_P targets from the other side, as described previously in Smith et al., Nucleic Acids Research, 2006, 34, e149; International PCT Applications WO 2007/049095, WO 2007/057781, WO 2007/049156, WO 2007/060495, WO 2006/097784 and WO 2006/097853; Arnould et al., J. Mol. Biol., 2006; 355, 443-458). Coexpression of the cutters as described previously (International PCT Application WO 2006/097854 and Arnould et al., J. Mol. Biol., 2006, 355, 443-458) leads then to a strong cleavage of the RAG1.10 target. The M2/M3 RAG1.10 heterodimer gives the strongest cleavage in yeast (International PCT Application WO 2008/010093). M2 is a RAG1.10.2 cutter and bears the following mutations in comparison with the I-CreI wild type sequence: N30R, Y33N, Q44A, R68Y, R70S and I77R. M3 is a RAG1.10.3 cutter and bears the following mutations in comparison with the I-CreI wild type sequence: K28N, Y33S, Q38R, S40R, Q44Y, R70S, D75Q and I77V.

Following the same experimental scheme as in Examples 4 and 5, three single chain constructs were engineered using the two linkers L1 and RM2 (see Table 1 of Example 4), resulting in the production of the three single chain molecules: M2-L1-M3, M2-RM2-M3 and M3-RM2-M2. In a second step, the G19S mutation was introduced in the N-terminal M2 mutant from the M2-L1-M3 and M2-RM2-M3 single chain molecules, resulting in two additional constructs. In addition, mutations K7E, K96E were introduced into the M3 mutant and mutations E8K, E61R into the M2 mutant of M3-RM2-M2 to create the single chain molecule: M3(K7E K96E)-RM2-M2(E8K E61R) that is called further SC_OH The six single chain constructs were then tested in yeast for cleavage of the RAG1.10 target and of its two RAG1.10.2 and RAG1.10.3 derivatives.

1) Material and Methods

See example 4, except for mutations of I-CreI CLS (T42A, E110W and Q111R) which were reverted.

Cloning of the SC_OH Single Chain Molecule

A PCR reaction was performed on the M2 mutant carrying the K7E and K96E mutations cloned in the pCLS0542 yeast expression vector. The PCR reaction uses the reverse primer CreCterSacI (5′-tagacgagctcctacggggaggatttatatctcgct-3′; SEQ ID NO: 94) and the forward primer. RM2 (5′-tatcggccggtggatctgataagtataatcaggctctgtctaaatacaaccaagcactgtccaagtacaatc aggccctgtctggtggaggcggttccaacaaagagttcctgctgtatcttgctggattt-3′; SEQ ID NO: 82).

The PCR fragment was purified and digested by EagI and SacI and ligated into the yeast expression vector for the M3 mutant carrying the mutations E8K and E61R also digested with EagI and SacI. After sequencing of the clones, a SC_OH single chain molecule was obtained

2) Results

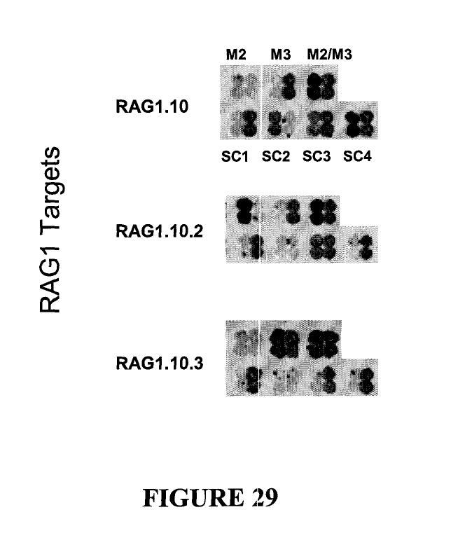

The activity of the four RAG1 single chain molecules (M2-L1-M3, M2_{G19S}-L1-M3, M2-RM2-M3 and M2_{G19S}-RM2-M3) was monitored against the three RAG1 targets RAG1.10, RAG1.10.2 and RAG1.10.3 (FIG. 29) using the previously described yeast assay (International PCT Application WO 2004/067736; Epinat et al., Nucleic Acids Res., 2003, 31, 2952-2962; Chames et al., Nucleic Acids Res., 2005, 33, e178, and Arnould et al., J. Mol. Biol., 2006, 355, 443-458). As observed previously in examples 4 and 5, the RM2 linker seems to be better adapted to the way the single chain constructs were engineered: cleavage of the RAG1.10 target was observed with the M2-RM2-M3 molecule, but not with M2-L1-M3. In addition, M2-RM2-M3 was found to cleave also RAG1.10.2. Since M3 does not cleave RAG1.10.2 (FIG. 29), these results suggest that intermolecular interactions can still result in contact between two M2 units, that would form a homodimeric or pseudo-homodimeric species responsible for cleavage of the palindromic RAG1.10.2 target.

Introduction of the G19S mutation in the M2 mutant improved the activity of both molecule, since M2_{G19S}-L1-M3 cleaves the RAG1.10 target, and M2_{G19S}-RM2-M3 is more active than M2-L1-M3. In addition, the G19S mutation, which has been shown previously to impair formation of functional homodimers (see example 4 of the present Application and International PCT Application WO 2008/010093), abolishes RAG1.10.2 cleavage. This result is consistent with the hypothesis that interaction between M2 units from distinct M2-RM2-M3 single chain molecules is still possible. However, the single chain structure might favour intramolecular interactions in some extent, for in contrast with M3, the M2-RM2-M3 molecule does not cleave RAG1.10.3 In conclusion, the M2_{G19S}-RM2-M3 RAG1 single chain molecule cleaves the RAG1.10 target at a saturating level in yeast, comparable to that observed with the M2/M3 heterodimer, and does not show any cleavage of the two derived palindromic targets RAG1.10.2 and RAG1.10.3.

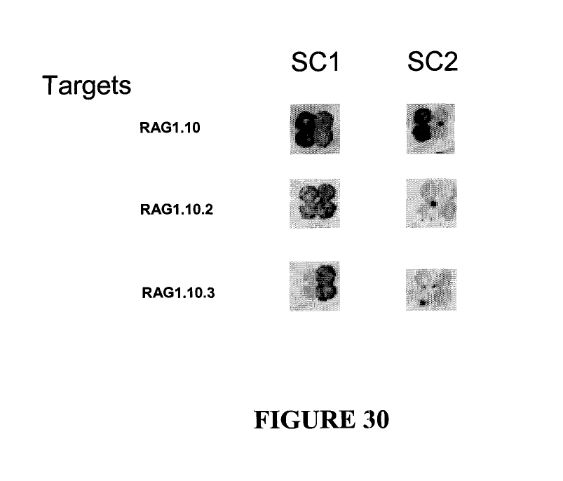

The yeast screen of the two single chain molecules M3-RM2-M2 and SC_OH against the three RAG1.10 targets depicted in FIG. 30 shows that introduction of the K7E/E8K and E61R/K96E allows for the abolition of the homodimeric activity against the RAG1.10.2 target without reducing the single chain cleavage activity for the RAG1.10 target. It is therefore possible to introduce these mutations in a single chain molecule to improve its specificity without affecting its activity toward the DNA target of interest.

### Example 7

### Making of Rag1 Single Chain Molecules with Different Positions for the Two Mutants Composing the Single Chain Molecules and Different Localization of the G19S Mutation

To evaluate the impact of the localization of each mutant and of the G19S mutation on the single chain molecule activity, six constructs (M2-RM2-M3, M2_{G19S}-RM2-M3, M2-RM2-M3_{G19S}, M3-RM2-M2, M3_{G19S}-RM2-M2, M3-RM2-M2_{G19S}) were tested for cleavage against the three RAG1.10, RAG1.10.2 and RAG1.10.3 targets using our yeast screening assay.

1) Material and Methods

See example 4

2) Results

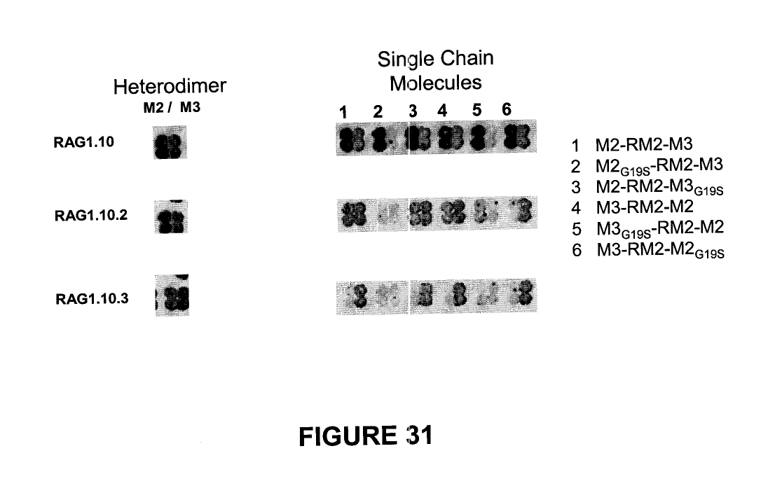

As shown in FIG. 31 all six single chain molecules (M2-RM2-M3, M2_{G19S}-RM2-M3, M2-RM2-M3_{G19S}, M3-RM2-M2, M3_{G19S}-RM2-M2, M3-RM2-M2_{G19S}) are highly active on the RAG1.10 target, activity equivalent to the initial M2/M3 heterodimer activity on the same target. The relative position (N-ter or C-ter) of the monomer M2 or M3 in the Single Chain does not influence the overall activity of the molecule. For example, the M2-RM2-M3 protein shows the same cleavage pattern as the M3-RM2-M2 molecule, cleaving with the same intensity the RAG1.10 and RAG1.10.2 targets but not the RAG1.10.3 target (RR target).

For some constructs, the RAG1.10.2 palindromic target is cleavable suggesting that inter-molecular interactions can occur and create active nuclease. However, this residual activity is abolished by the introduction of the G19S mutation. The comparison of the activity profile of molecules 2 and 3 in FIG. 31 (M2G19S-RM2-M3 and M2-RM2-M3G19S) shows that the G19S mutation localization does not influence activity on the RAG1.10 target but abolishes a residual homodimer activity, when the mutant (here mutant M2) responsible for this activity carries the G19S mutation.

### Example 8

### Importance of the G19S Mutation in a Single Chain Molecule as Shown by Extrachromosomal Assay in Cho Cells

In terms of RAG1.10 cleavage activity, the six single chain molecule presented above as well as the SC_OH single chain molecule depicted in example 6 seem to be equivalent in the yeast screening assay. But they all cleave this target at saturating levels. Therefore, activity of two single chain molecules with or without the G19S mutation against the three RAG1.10, RAG1.10.2 and RAG1.10.3 targets was evaluated in CHO cells using an extrachromosomal SSA assay described in example 4. The four single chain molecules chosen for this assay were: M3-RM2-M2, M3-RM2-M2_{G19S}, M3⁻-RM2-M2⁺ and M3⁻-RM2-M2⁺_{G19S}. M3⁻ indicates the M3 mutant carrying the K7E and K96E mutations and M2⁺ represents the M2 mutant carrying the E8K and E51R mutations.

1) Material and Methods

Cloning of M3⁻-RM2-M2⁺ and M3⁻-RM2-M2⁺_{G19S} Molecules into a Mammalian Expression Vector

The methodology is exactly the same as the one described in example 4 but the CCM2For primer was replaced by the CCM2ForE8K primer (5′-AAGCAGAGCTCTCTGGCTAACTAGAGAACCCACTGCTTACTGGCTTATCGACCA TGGCCAATACCAAATATAACAAAAAGTTCC-3′: SEQ ID NO: 109).

2) Results

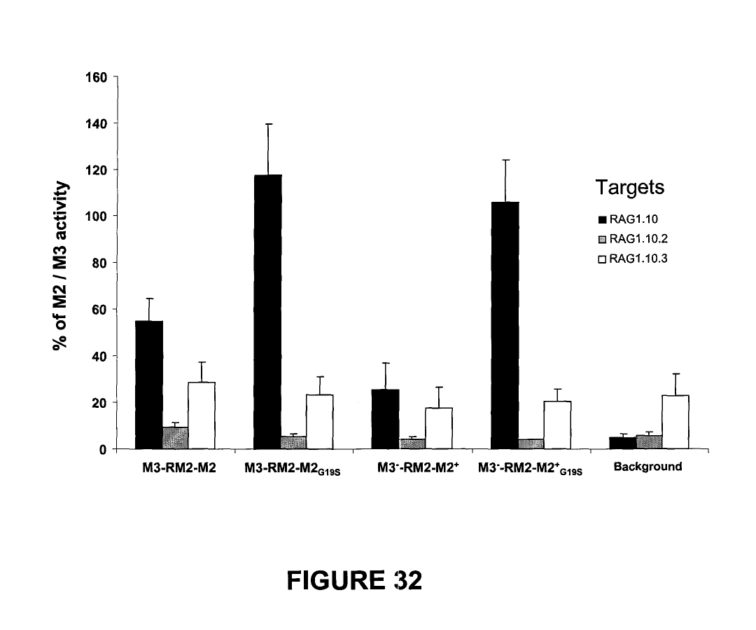

FIG. 32 shows the extrachromosomal SSA activity in CHO cells for the four single chain molecules M3-RM2-M2, M3-RM2-M2_{G19S}, M3⁻-RM2-M2⁺ and M3⁻-RM2-M2⁺_{G19S}, against the three RAG1.10, RAG1.10.2 and RAG1.10.3. Activity against the three targets is represented as a percentage of the activity of the initial M2/M3 heterodimer against these same three targets. The four single chain proteins have homodimer activities (against the RAG1.10.2 and RAG1.10.3 targets) equivalent to the background level. FIG. 32 shows also that the G19S mutation is essential to the single chain molecule activity as only the two single chain proteins carrying the G19S mutation present a RAG1.10 target cleavage activity equivalent or even greater than the M2/M3 heterodimer activity.

### Example 9

### Rag1 Single Chain Molecules Induce High Levels of Gene Targeting in CHO-K1 Cells

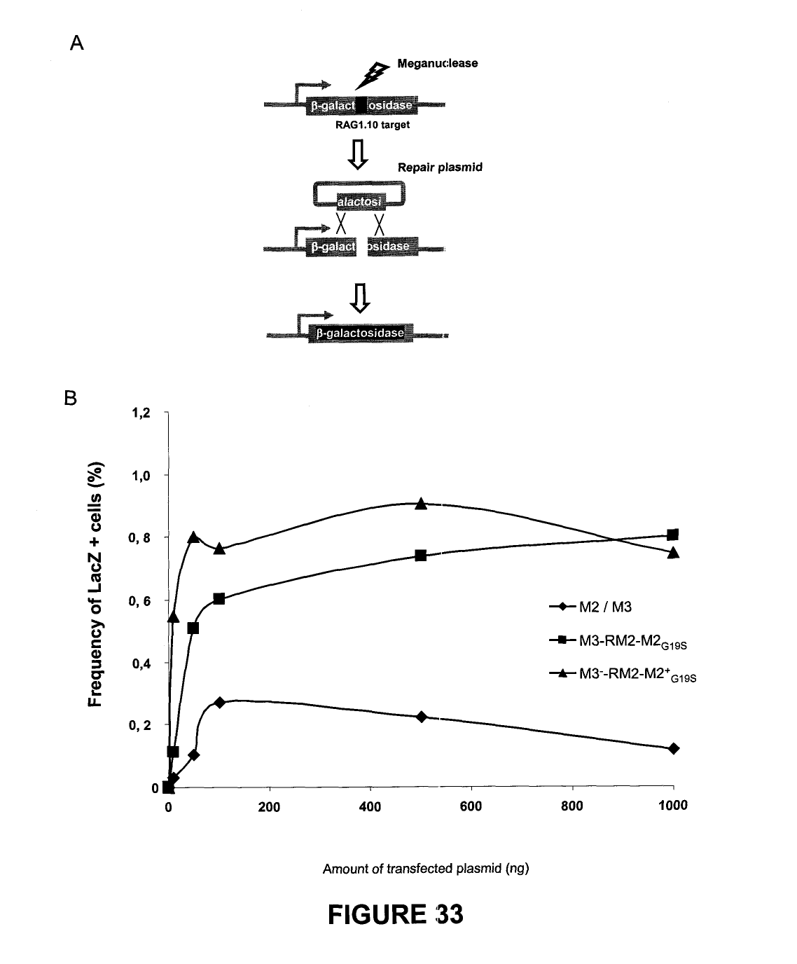

To further assess the cleavage activity of the two single chain molecules M3-RM2-M2_{G19S} (SEQ ID NO: 111 encoded by the nucleotide sequence SEQ ID NO: 110) and M3⁻-RM2-M2⁺_{G19S} (SEQ ID NO: 113 encoded by the nucleotide sequence SEQ ID NO: 112), a chromosomal reporter system in CHO cells was used (FIG. 33). In this system a single-copy LacZ gene driven by the CMV promoter is interrupted by the RAG1.10 sequence and is thus non-functional. The transfection of the cell line with plasmids coding for RAG1 single chain meganucleases and a LacZ repair plasmid allows the restoration of a functional LacZ gene by homologous recombination. It has previously been shown that double-strand breaks can induce homologous recombination; therefore the frequency with which the LacZ gene is repaired is indicative of the cleavage efficiency of the genomic RAG1.10 target site.

1) Material and Methods

Chromosomal Assay in CHO-K1 Cells

CHO-K1 cell lines harbouring the reporter system were seeded at a density of 2×10⁵ cells per 10 cm dish in complete medium (Kaighn's modified F-12 medium (F12-K), supplemented with 2 mM L-glutamine, penicillin (100 UI/ml), streptomycin (100 μg/ml), amphotericin B (Fongizone) (0.25 μg/ml) (INVITROGEN-LIFE SCIENCE) and 10% FBS (SIGMA-ALDRICH CHIMIE). The next day, cells were transfected with Polyfect transfection reagent (QIAGEN). Briefly, 2 μg of lacz repair matrix vector was co-transfected with various amounts of meganucleases expression vectors. After 72 hours of incubation at 37° C., cells were fixed in 0.5% glutaraldehyde at 4° C. for 10 min, washed twice in 100 mM phosphate buffer with 0.02% NP40 and stained with the following staining buffer (10 mM Phosphate buffer, 1 mM MgCl₂, 33 mM K hexacyanoferrate (III), 33 mM K hexacyanoferrate (II), 0.1% (v/v) X-Gal). After, an overnight incubation at 37° C., plates were examined under a light microscope and the number of LacZ positive cell clones counted. The frequency of LacZ repair is expressed as the number of LacZ+ foci divided by the number of transfected cells (5×10⁵) and corrected by the transfection efficiency.

2) Results

FIG. 33 shows that the two single chain molecules M3-RM2-M2_{G19S} and M3⁻-RM2-M2⁺_{G19S} can induce high level of gene targeting in CHO cells. They even increase the gene correction frequency by a 3.5 fold factor in comparison with the initial M2/M3 heterodimer.

### Example 10

### Making of a Rag1 Single Chain Molecule with Different N- and C-Terminal Endings for Both Subunits

Using the M2-RM2-M3 RAG1 single chain molecule, new single chain constructs with different N- and C-terminal endings for both subunits were engineered.

These new constructs could allow to pinpoint the best possible position of the linker joining the two I-CreI derived mutants. The N-terminal of I-CreI consists in a 6 residues loop followed by the LAGLIDADG α-helix starting at residue K7. In the I-CreI structure (PDB code 1G9Y), the last 10 C-terminal residues are not visible because probably disordered. In the structure, the C-terminus ends at residue D153 with the helix α6 covering residues 145 to 150. So several single chain constructs were made where the N-terminus of the M2 or M3 mutant begins with the residue M1, N2 or N6 and C-terminus ends at different positions, respectively S145, L152, S156 and K160. Activity of these different RAG1 single chain constructs was monitored using the previously described yeast screening assay (see example 4).

1) Material and Methods

a) Cloning of Truncated Versions of the M2 Mutant in the Yeast Expression Vector

Cloning of the RAG1 M2-RM2-M3 single chain requires first to get the M2 mutant cloned in the yeast expression vector (pCLS0542) (see Material and Methods of Example 6). To clone truncated versions of the M2 mutant, several PCR reactions were performed with different primers couples: CreNter6/CreCter, CreNter/CreCter160, CreNter/CreCter156, CreNter/CreCter152, CreNter/CreCter145. Sequences of the different primers are listed in the Table 2 below. The different PCR fragments were then digested with the restriction enzymes NcoI and EagI, and ligated into the pCLS0542 vector also digested with NcoI and EagI. The clones were then sequenced. The truncated versions of the M2 mutant are respectively: M2 (6-163), M2(1-160), M2(1-156), M2(1-152) and M2(1-145), where the numbers indicate the I-CreI residues contained in the M2 mutant coding sequence.

b) Cloning of a Rag1 Single Chain Molecule with Different Endings for Both Subunits

Different PCR reactions were performed on the M3 mutant in the I-CreI CLS version. Each PCR reaction uses one forward primer and one reverse primer. There are two possible forward primers (RM2 and RM2N2) and five possible reverse primers (CreCterR60, Cre160R60, Cre156R60, Cre152R60 and Cre145R60). The forward primers allow to obtain a M3 coding sequence beginning at residue N6 or N2 and the reverse primers allow to obtain a M3 coding sequence ending respectively at residues P163, K160, S156, L152 and S145. The different PCR fragments were purified and digested by EagI and SacI and each PCR fragment was ligated into the yeast expression vector for one of the M2 mutants described above also digested with EagI and SacI. After sequencing of the clones, all possible single chain molecules in the yeast expression vector were obtained.

TABLE 2

Primers sequences

| | Sequence |
|---|---|
| Primer Name | (SEQ ID NO: 95 to 108) |
| CreNter | 5′- acaggccatggccaataccaaatataacaaag -3′ |
| CreNter6 | 5′- acaggccatggccaacaaagagttcctgctgtacctg-3′ |
| CreCter | 5′- gattgacggccgccggggaggatttcttctt-3′ |
| CreCter160 | 5′- gattgacggccgctttcttcttctcgctcaggctgtc -3′ |
| CreCter156 | 5′- gattgacggccgcgctcaggctgtccaggacagcacg -3′ |
| CreCter152 | 5′- gattgacggccgccaggacagcacgaacggtttcaga -3′ |
| CreCter145 | 5′- gattgacggccgcagaagtggttttacgcgtcttaga -3′ |
| RM2 | 5′- tatcggccggtggatctgataagtataatcaggctctgtctaaatacaaccaagcactgtccaa |
| | gtacaatcaggccctgtctggtggaggcggttccaacaaagagttcctgctgtatcttgctggattt -3′ |
| RM2N2 | 5′- tatcggccggtggatctgataagtataatcaggctctgtctaaatacaaccaagcactgtccaagta |
| | caatcaggccctgtctggtggaggcggttccaacaccaagtacaacaaagagttcctgctgtat -3′ |
| CreCterR60 | 5′- tagacgagctcctaaggagaggactttttcttctcag -3′ |
| Cre160R60 | 5′- tagacgagctcctactttttcttctcagagaggtcatc -3′ |
| Cre156R60 | 5′- tagacgagctcctaagagaggtcatccagaactgccct -3′ |
| Cre152R60 | 5′- tagacgagctcctacagaactgccctcacagtctcaga -3′ |
| Cre145R60 | 5′- tagacgagctcctaagaggtggtttttctggtcttgga -3′ |

2) Results

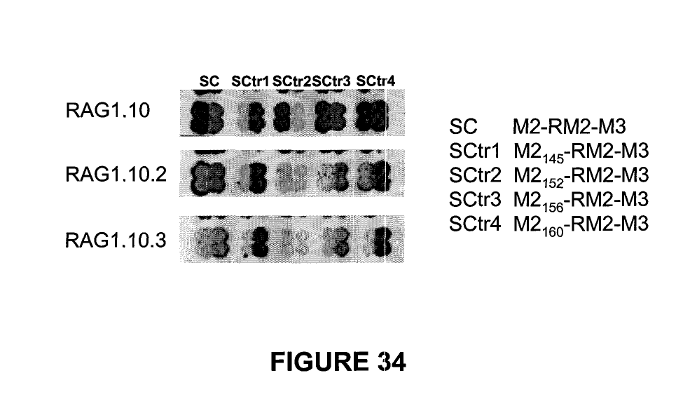

Starting from the RAG1 M2-RM2-M3 single chain molecule, four new single chain molecules were built where the N-Terminal M2 mutant was truncated at its C-terminus. These proteins are called SCtr1, SCtr2, SCtr3 and SCtr4 and correspond to single chain molecules, where the M2 mutant sequence ends respectively at residue 145, 152, 156 and 160. These four molecules as well as the initial M2-RM2-M3 single chain were tested for cleavage toward the three RAG1.10, RAG1.10.2 and RAG1.10.3 targets using our yeast screening assay. FIG. 34 shows that SCtr1 is inactive against the three targets and that the three other truncated versions of the single chain cleave the RAG1.10 target and slightly the RAG1.10.2 target. This result suggests that residues 145 to 152 of the M2 mutant that form the helix α6 are necessary to the single chain activity.

### Example 11

### Gene Targeting at the Endogenous Rag1 Locus in Human Cells

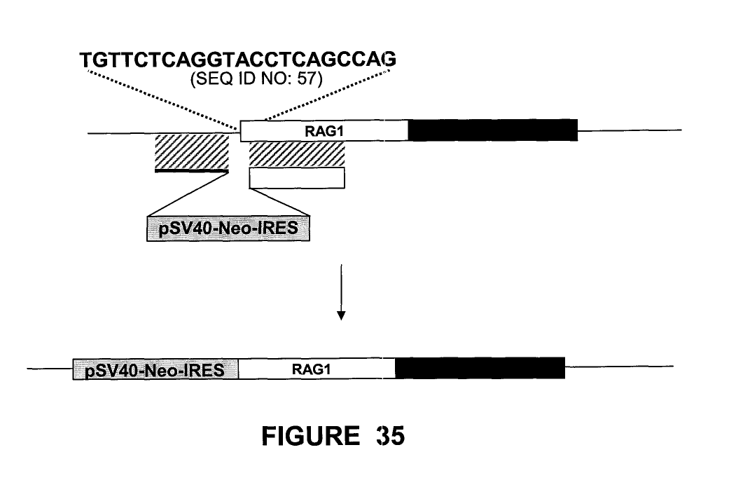

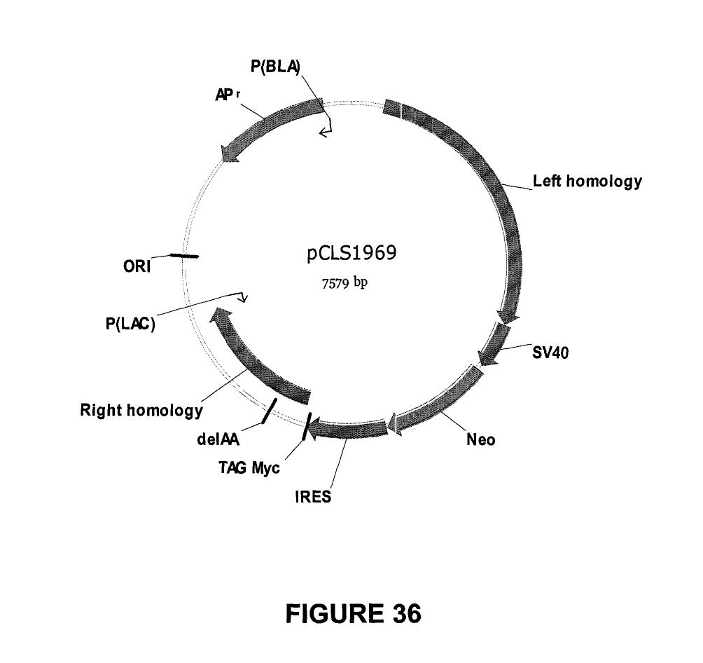

To further validate the cleavage activity of engineered single-chain Rag1 meganucleases, their ability to stimulate homologous recombination at the endogenous human RAG locus was evaluated (FIG. 35). Cells were transfected with mammalian expression plasmids for one of two single chain molecules M3-RM2-M2_{G19S} (SEQ ID NO: 111) and M3⁻-RM2-M2⁺_{G19S} (SEQ ID NO: 113) and the donor repair plasmid pCLS1969 (FIG. 36) containing 1.7 kb of exogenous DNA sequence flanked by two sequences, 2 kb and 1.2 kb in length, homologous to the human RAG1 locus. Cleavage of the native RAG1 gene by the meganuclease yields a substrate for homologous recombination, which may use the donor repair plasmid containing 1.7 kb of exogenous DNA flanked by homology arms as a repair matrix. Thus, the frequency with which targeted integration occurs at the RAG1 locus is indicative of the cleavage efficiency of the genomic RAG1.10 target site.

1) Materials and Methods

a) Meganuclease Expression Plasmids

The Rag1 meganucleases used in this example are M3-RM2-M2_{G19S} (SEQ ID NO: 111) and M3⁻-RM2-M2⁺_{G19S} (SEQ ID NO: 113) cloned in a mammalian expression vector, pCLS1088 (FIG. 17).

b) Donor Repair Plasmid

The donor plasmid for gene targeting experiments contains a PCR generated 2075 by fragment of the RAG1 locus (position 36549341 to 36551416 on chromosome 11, NC_{—}000011.8) as the left homology arm and a 1174 bp fragment of the RAG1 locus (position 36551436 to 36552610 on chromosome 11, NC_{—}000011.8) as the right homology arm. An exogenous 1.8 kb DNA fragment containing an SV40 promoter, neomycin resistance gene and an IRES sequence was inserted in between the two homology arms using EcoRI and BamHI sites that were introduced during the PCR amplification of the RAG1 locus. The resulting plasmid is pCLS1969 (FIG. 36).

c) Rag1 Gene Targeting Experiments

Human embryonic kidney 293H cells (Invitrogen) were plated at a density of 1×10⁶ cells per 10 cm dish in complete medium (DMEM supplemented with 2 mM L-glutamine, penicillin (100 UI/ml), streptomycin (100 μg/ml), amphotericin B (Fongizone) (0.25 μg/ml) (Invitrogen-Life Science) and 10% FBS). The next day, cells were transfected with Lipofectamine 2000 transfection reagent (Invitrogen) according to the supplier's protocol. Briefly, 2 μg of the donor plasmid was co-transfected with 3 μg of single-chain meganuclease expression vectors. After 72 hours of incubation at 37° C., cells were trypsinized and plated in complete medium at 10 cells per well in a 96-well plate or at 200 cells in a 10 cm plate. Individual clones were subsequently picked from the 10 cm plate using a ClonePix robot (Genetix). Genomic DNA extraction was performed with the ZR-96 genomic DNA kit (Zymo research) according to the supplier's protocol.

d) PCR Analysis of Gene Targeting Events

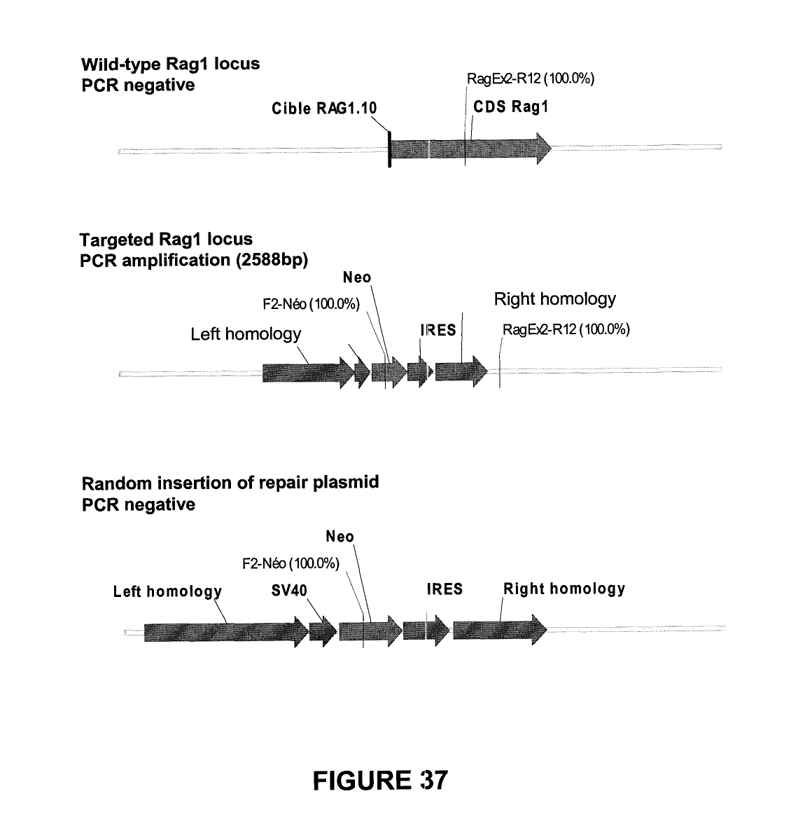

The frequency of gene targeting was determined by PCR on genomic DNA using the primers F2-neo: 5′-AGGATCTCCTGTCATCTCAC-3′ (SEQ ID NO: 114) and RagEx2-R12: 5′-CTTTCACAGTCCTGTACATCTTGT-3′ (SEQ ID NO: 115) that result in a 2588 bp gene targeting specific PCR product (FIG. 37). The F2 primer is a forward primer located in the neomycin coding sequence. The R12 primer is a reverse primer located in the human RAG1 gene outside of the right homology arm of the donor repair plasmid.

e) Southern Blot Analysis

Southern blot analysis was performed with genomic DNA digested with HindIII and hybridized with an 830 by RAG1 specific probe that is 3′ of the right homology arm of the donor repair plasmid.

2) Results

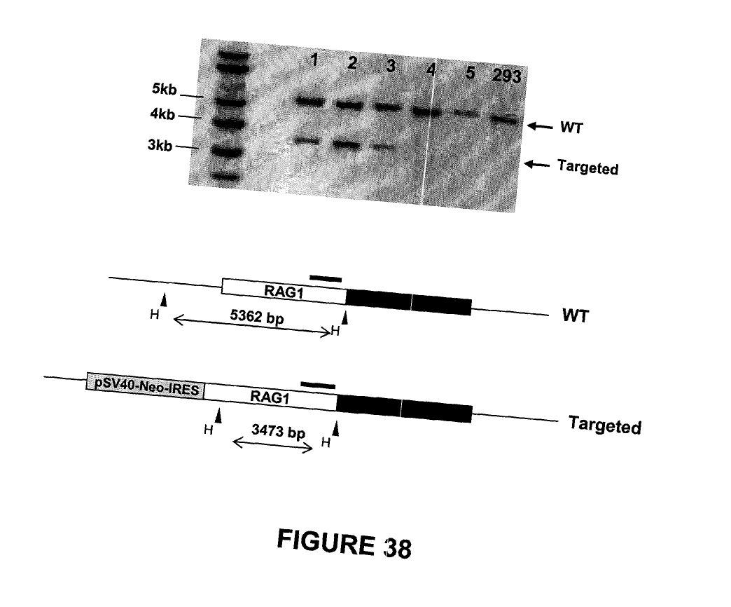

Human embryonic kidney 293H cells were co-transfected with 2 vectors: a plasmid expressing one of the two single-chain Rag1 meganucleases and the donor repair plasmid pCLS1969 (FIG. 36). As a control for spontaneous recombination, 293H cells were also transfected with the donor repair plasmid alone. The cells were then plated at 10 cells per well in a 96-well microplate or plated in 10 cm dishes and individual clones subsequently picked. Genomic DNA derived from these cells was analyzed for gene targeting by PCR as described in Material and Methods. Results are presented in Table 3. In the absence of meganuclease (repair plasmid alone), no PCR positive signal was detected among the 2,560 cells analyzed in pools of 10 cells or the 94 individual clones examined. In contrast, in the presence of the M3-RM2-M2_{G19S} meganuclease, 11 positives were detected among the 2,560 cells analyzed in pools of 10 cells indicating a frequency of recombination of 0.4%. Among the 94 individual clones examined, none were positive. In the presence of M3⁻-RM2-M2⁺_{G19S}, 34 positive clones (1.4%) were detected among 2560 cells (in pools of 10) analyzed, and among the 94 individual clones analyzed, 4 were positive (4.3%). Southern blot analysis of these 4 individual clones indicates that three are consistent with a gene targeting event at the Rag1 locus (FIG. 38). These results demonstrate that the two single chain molecules M3-RM2-M2_{G19S} and M3⁻-RM2-M2⁺_{G19S} are capable of inducing high levels of gene targeting at the endogenous Rag1 locus.

TABLE 3
Frequency of gene targeting events at the Rag1 locus
in human 293H cells

| | | | Gene targeting |
|---|---|---|---|
| Meganuclease | Cells per well | PCR+ events | frequency |
| M3-RM2-M2_{G19S} | 10 | 11/2510 | 0.4% |
| M3-RM2-M2_{G19S} | 1 | 0/94 | NA |
| M3⁻-RM2-M2⁺_{G19S} | 10 | 34/2430 | 1.4% |
| M3⁻-RM2-M2⁺_{G19S} | 1 | 4/94 | 4.3% |
| None | 10 | 0/2560 | NA |
| None | 1 | 0/94 | NA |

NA: not applicable

### Example 12

### The I-CreI Derived Single Chain Meganuclease is Stable

1) Material and Methods

a) Protein Expression and Purification

In order to coexpress the heterodimeric I-CreI derivatives each of the monomers ORF were cloned into CDFDuet-1 vector (NOVAGEN) with a 6×His tag or a Strep tag at the C-terminus and purification of the double tagged heterodimers was performed as described previously (P. Redondo et al., Nature, 2008, 456, 107-1). The single-chain M3-RM2-M2_{G19S} protein sequences (example 7) with a His₆ tag at the C-terminus was cloned in a pET24d(+) vector and expressed in E. coli Rosetta(DE3)pLysS cells (NOVAGEN) grown in LB plus kanamycin and chloramphenicol. Induction with IPTG for 5 h at 37° C. or for 15 h at 20° C. yielded high expression levels, however, after sonication in lysis buffer containing 50 mM sodium phosphate pH 8.0, 300 mM NaCl, 5% glycerol and protease inhibitors (Complete EDTA-free tablets, ROCHE) and ultracentrifugation at 20,000 g for 1 hour, the protein was found exclusively in the insoluble fraction as detected by a western blot with an anti-His antibody. Thus the protein was purified under denaturing conditions by first solubilizing it in lysis buffer plus 8 M urea. After clarification by ultracentrifugation (2 h at 40,000 g) the sample was applied onto a column packed with Q-Sepharose XL resin (GE Healthcare) equilibrated in the same buffer. This purification step separated all the nucleic acids (retained in the column) from the protein and improved the performance of the subsequent purification steps. The protein is recovered from the flowthrough by means of a Co₂₊-loaded HiTrap Chelating HP 5 ml column (GE Healthcare) equilibrated in the lysis buffer plus 8 M urea. After sample loading and column washing the protein is eluted with the same buffer plus 0.5 M imidazol. Protein-rich fractions (determined by SDS-PAGE) were collected and refolded by a 20 fold dilution (drop by drop) into 20 mM sodium phosphate pH 6.0 300 mM NaCl at 4° C. (final protein concentration of 0.13 mg/ml). The refolded protein was loaded onto a 5 ml HiTrap heparin column equilibrated in the same buffer and eluted with a gradient to 1 M NaCl. The fractions with pure protein were pooled, concentrated up to 1.4 mg/ml (35.6 nM, determined by absorbance at 280 nm) and were either used immediately or flash frozen in liquid nitrogen and stored at −80° C.

b) Biochemical and Biophysical Characterization of Proteins

Circular dichroism (CD) measurements were performed on a Jasco J-810 spectropolarimeter using a 0.2 cm path length quartz cuvette adm 10-4 μM protein solutions in phosphate saline buffers. Equilibrium unfolding was induced increasing temperature at a rate of 1° C./min (using a programmable Peltier thermoelectric). Analytical gel filtration chromatography was performed at room temperature with an AKTA FPLC system (GE) using a Superdex 200 10/300GL column in 20 mM sodium phosphate buffer pH 6.0 1 M NaCl. A sample of 100 μL of SC-v3v2G19S constructs at a concentration of 0.3 mg/ml was injected and eluted at a flow rate of 0.2 ml/min. The column was calibrated with blue dextran (excluded volume) and molecular weight markers from 17 to 670 kDa (BioRad).

2) Results

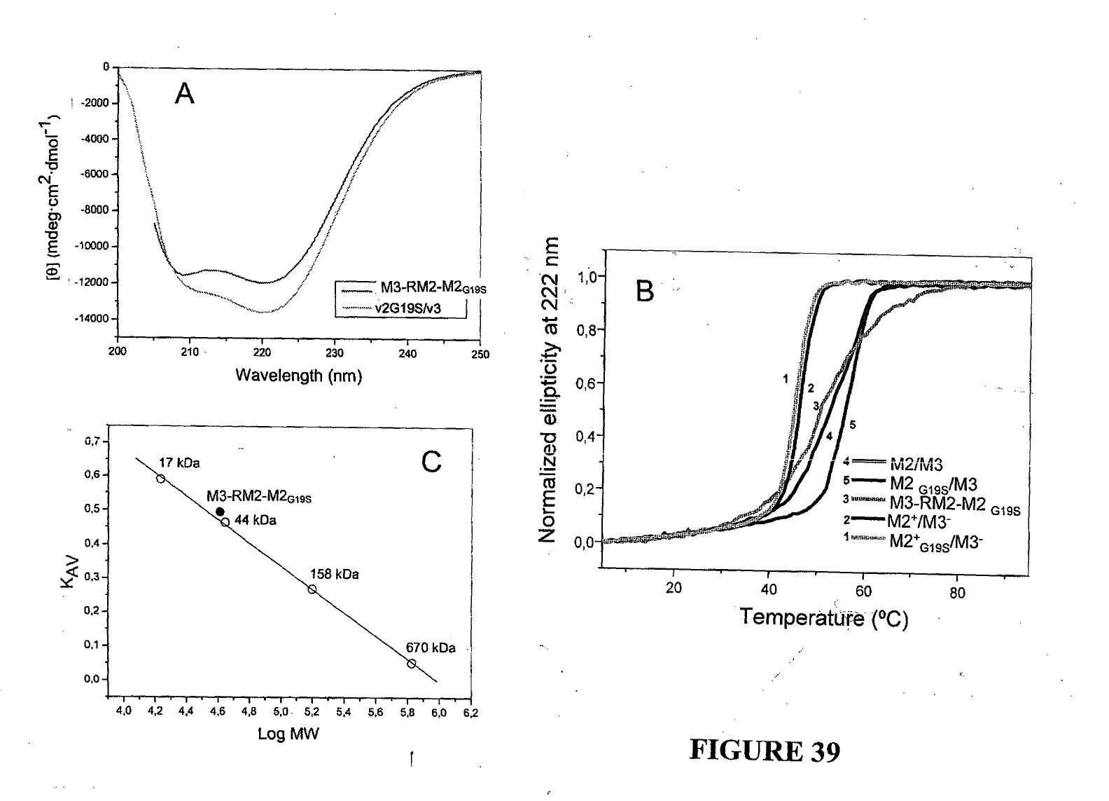

The protein identity was confirmed by mass spectrometry which showed that the initial methionine was absent in the purified polypeptide chain. The purified proteins were found to be folded with a similar structure as the wild type by circular dichroism and NMR and to be dimeric in solution by analytical ultracentrifugation and gel filtration. The structure and stability of the single-chain molecule were very similar to those of the heterodimeric variants and this molecule appeared to be monomeric in solution (FIG. 39).

### Example 13

### The I-CreI Derived Single Chain Meganuclease is not Toxic

1) Material and Methods

a) Cell Survival Assay

CHO-K1 cell line was seeded at a density of 2×10⁵ cells per 10 cm dish. The next day, various quantity of meganuclease expression vectors and a constant amount of plasmid coding for GFP were co-transfected into CHO-K1 cells in 10 cm plate. GPF expression was monitored at days 1 and 6 post-transfection by flow cytometry (Guava EasyCyte, Guava technologies). Cell survival corrected by the transfection efficiency measured at day 1 was calculated as a ratio of [(meganuclease transfected cell expressing GFP at day 6)/(meganuclease transfected cell expressing GFP at day1)]/[(control transfected cell expressing GFP at day 6)/(control transfected cell expressing GFP at day1).

b) γH2AX Immunocytochemistry

For γH2AX immunocytochemistry, CHO-K1 cells were transfected by Polyfect reagent (Qiagen) with a 4 μg of DNA mixture containing different amounts of plasmid encoding a HA-tagged meganuclease and completed to 4 μg with empty vector as a stuffer. 48 h after transfection, cells were fixed with 2% of paraformaldehyde for 30 minutes and permeabilized with 0.5% Triton for 5 nm at RT. After wash, cells were incubated with PBS/triton 0.3% buffer containing 10% normal goat serum (NGS) and 3% BSA for 1 hour to block nonspecific staining. Cells were then incubated one hour at RT with anti-γH2AX (Upstate: 1/10000) and anti-HA (Santa Cruz: 1/100) antibodies diluted in PBS/triton 0.3% with 3% BSA and 10% NGS followed by 1 hour incubation with secondary antibody Alexa Fluor 488 goat antimouse (Invitrogen-Molecular probes: 1/1000) and Alexa Fluor 546 goat anti-rabbit diluted in PBS/triton 0.3%, 3% BSA, and 10% NGS. After incubation with 1 μg/ml 4,6-diamino-2-phenyindole (DAPI, Sigma), coverslips were mounted and the γH2AX foci were visualized in transfected (HA positive) cells by fluorescent microscopy.

2) Results

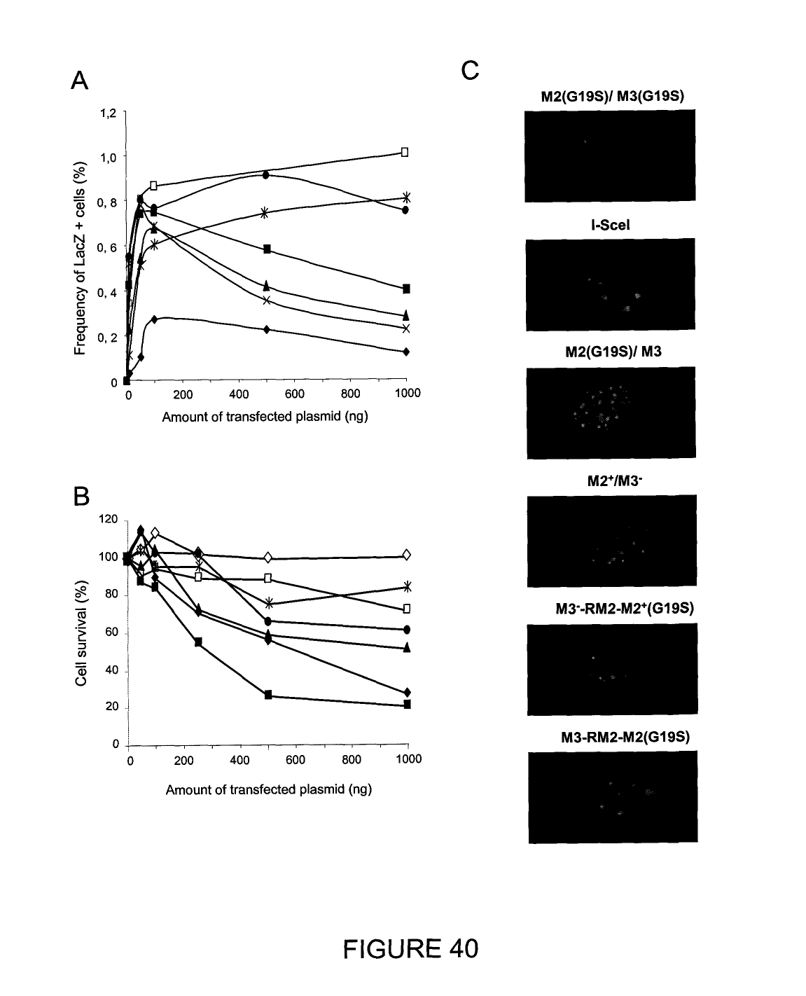

Toxicity is a major issue for DSB-induced recombination technology, particularly for therapeutic applications, for which the activity/toxicity ratio is of major concern. The toxicity of the RAG1 meganucleases (examples 7 and 8) was evaluated in a cell survival assay, as previously described (M. L. Maeder et al., Mol. Cell., 2003, 31, 2952-). The link between efficacy and toxicity was investigated by adapting this assay for use with the CHO-K1 cells used for the activity dose response assay. At the active dose (the dose at which the meganucleases displayed their maximum level of activity FIG. 40A), toxicity was barely detectable, regardless of the meganuclease used (FIG. 40B). However, the M2/M3 heterodimer displayed significant toxicity at high doses, which was partly alleviated by the obligatory heterodimer design. The single-chain design also represented an improvement, as the best version reproduced the pattern obtained with I-SceI. The toxicity of sequence-specific endonucleases is usually attributed to off-site cleavage, which can result in mutations, deletions, translocations and other gross genomic alterations (M. H. Porteus, D. Carroll, Nat. Biotechnol., 2005, 23, 967-). The off-site cleavage, was evaluated by monitoring the formation of phosphorylated H2AX histone (γ-H2AX) foci. γ-H2AX focus formation is one of the first responses of the cell to DNA double-strand breaks (DSBs) and provides a convenient means of monitoring DSBs in living cells (E. P. Rogakou et al., J. Biol. Chem., 1998, 273, 5858). CHO-K1 cells were transfected with two doses of meganuclease expression vectors (active dose and 10-fold excess). The M2(G19S)/M3(G19S) heterodimer was used as a control for non toxicity, as this molecule was completely inactive in the assays. At the active dose, no signal above background was observed, regardless of the meganuclease variant used, consistent with published results (P. Redondo et al., Nature, 2008, 456, 107-. In accordance with previous data, the M2(G19S)/M3 heterodimer, when used at ten times the active dose, induced significant levels of focus formation, as illustrated in FIG. 40C, whereas use of either the obligatory heterodimer or the single-chain design minimized off-site cleavage. Ultimately, the signals obtained with the single-chain molecule M3-RM2-M2⁺_{G19S} were similar to those obtained with the I-SceI meganuclease.

TABLE 4

| | | | | | | Acces N^{o} | | Acces | | |
|---|---|---|---|---|---|---|---|---|---|---|
| Protein | Synonym | Organism | Species | Size (kD) | Motif | (mega) | Acces N^{o} (gene) | N^{o} (genome) | Year | Reference |
| F-SceI | Endo.SceI | Saccharomyces | cerevisiae | 476 | DD | M63839 | | | 1991 | Nakagawa et al. J. Biol. Chem. 266: 1977-1984 |
| F-SceII | HO | Saccharomyces | cerevisiae | 586 | DD | M14678 | | | 1983 | Kostriken et al. Cell 35: 167-174 |
| I-AcaI | Aca1931m | Acanthamoeba | castellanii | 142 | D | AAA20591 | U03732 | NC_001637 | 1994 | Lonergan et al. J. Mol. Biol. 239 (4), 476-499 |
| I-AcaII | Aca1951m | Acanthamoeba | castellanii | 168 | D | AAA20592 | U03732 | NC_001637 | 1994 | Lonergan et al. J. Mol. Biol. 239 (4), 476-499 |
| I-AcaIII | Aca2593m | Acanthamoeba | castellanii | 164 | D | AAA20593 | U03732 | NC_001637 | 1994 | Lonergan et al. J. Mol. Biol. 239 (4), 476-499 |
| I-AstI | | Ankistrodesmus | stipitatus | 244 | D | L42984 | | | | |
| I-CagI | Cag2593c | Chlamydomonas | agloeformis | 246 | D | L43351 | | | 2001 | Lucas et al. NAR Feb 15; 29(4): 960-9 |
| I-CbrI | Cbr1931c | Chlorosarcina | brevispinosa | 153 | D | L49150 | | | 2001 | Lucas et al. NAR Feb 15; 29(4): 960-9 |
| I-CbrII | Cbr1951c | Chlorosarcina | brevispinosa | 163 | D | L49150 | | | 2001 | Lucas et al. NAR Feb 15; 29(4): 960-9 |
| I-CcaI | | Chlamydomonas | | | D? | | | | | |
| I-CecI | | Chlorococcum | echinozigotum | 213 | D | L44123 | | | | |
| I-CelI | | Chlorogonium | elongatum | 229 | D | L42860 | | | | |
| I-CeuI | | Chlamydomonas | eugametos | 218 | D | S14133 | Z17234 | | 1991 | Gauthier et al. Curr Genet 19: 43-7 |
| I-CeuII | I-CeuA II | Chlamydomonas | eugametos | 283 | D | | AF008237 | | 1998 | Denovan-Wright, et al. Plant Mol. Biol. 36: 285-95 |
| | I-CeuA IIP | | | | | | | | | |
| I-CfrI | Cfr1931c | Chlamydomonas | frankii | 154 | D | L43352 | | | 2001 | Lucas et al. NAR Feb 15; 29(4): 960-9 |
| I-CgeI | Cge1931c | Chlamydomonas | geitleri | 177 | D | L43353 | | | 2001 | Lucas et al. NAR Feb 15; 29(4): 960-9 |
| I-ChuI | | hlamydomonas | humicola | 218 | dd | L06107 | | | 1993 | Coté, et al. Gene 129: 69-76 |
| I-CiyI | Ciy2593c | Chlamydomonas | iyengarii | 212 | D | L43354 | | | 2001 | Lucas et al. NAR Feb 15; 29(4): 960-9 |
| I-CkoI | | Chlamydomonas | | | D? | | | | | |
| I-CluI | | Carteria | luzensis | 225 | D | L42986 | | | | |
| I-CluII | Clu2593c | Carteria | luzensis | 171 | D | L42986 | | | 2001 | Lucas et al. NAR Feb 15; 29(4): 960-9 |
| I-CmeI | Cme1931c | Chlamydomonas | mexicana | 140 | D | L49148 | | | 2001 | Lucas et al. NAR Feb 15; 29(4): 960-9 |
| I-CmoI | | Chlamydomonas | monadina | 216 | D | L49149 | | | | |
| I-CmuI | I-Cmoe I | Chlamydomonas | mutabilis | 219 | D | L42859 | | | | |
| I-ColI | Col2593c | Carteria | olivieri | 182 | D | L43500 | | | 2001 | Lucas et al. NAR Feb 15; 29(4): 960-9 |
| I-CpaI | | Chlamydomonas | pallidostigmatica | 152 | D | L36830 | | | 1995 | Turmel et al. Mol. Biol. Evol. 12: 533-45 |
| I-CpaIII | | Chlamydomonas | pallidostigmatica | 214 | D | L43503 | | | | |
| I-CreI | | Chlamydomonas | reinhardtii | 163 | D | X01977 | | | 1985 | Rochaix et al. NAR 13: 975-84 |
| I-CsmI | | Chlamydomonas | smithii | 237 | dd | X55305 | | | 1990 | Colleaux et al. Mol Gen Genet 223: 288-296 |
| I-CvuI | I-Cvu IP | Chlorella | vulgaris | 161 | D | L43357 | | | 1998 | Watanabe, et al. Gene 213: 1-7 |
| I-CvuII | Cvu1931m | Chlorella | vulgaris | 144 | D | | AY008337 | | 2001 | Lucas et al. NAR Feb 15; 29(4): 960-9 |
| I-CvuIII | Cvu1951m | Chlorella | vulgaris | 166 | D | | AY008338 | | 2001 | Lucas et al. NAR Feb 15; 29(4): 960-9 |
| I-DmoI | | Desulfurococcus | mobilis | 194 | DD | P21505 | | | 1985 | Kjems, et al. Nature 318: 675-77 |
| I-HlaI | Hla2593c | Haematococcus | lacustris | 166 | D | L49151 | | | 2001 | Lucas et al. NAR Feb 15; 29(4): 960-9 |
| I-Mso | Mso1931c | Monomastix | species | 138 | D | L49154 | | | 1995 | |
| I-Mso | Mso1931m | Monomastix | species | 202 | D | | AY008339 | | 1995 | |
| I-Mso | Mso1951c | Monomastix | species | 161 | D | L49154 | | | 1995 | |
| I-MsoI | | Monomastix | species | 170 | D | L49154 | | | 1995 | |
| I-Msp | Msp1931c | Monomastix | species | 140 | D | L44124 | | | 1995 | |
| I-Msp | Msp1931m | Monomastix | species | 150 | D | | AY008340 | | 1995 | |
| I-Msp | Msp1951c | Monomastix | species | 165 | D | L44124 | | | 1995 | |
| I-Msp | Msp2593c | Monomastix | species | 167 | D | L44124 | | | 1995 | |
| I-MviI | Mvi2593m | Mesostigma | viride | 162 | D | | AF323369 | | 2001 | Lucas et al. NAR Feb 15; 29(4): 960-9 |
| I-NcrIII | | Neurospora | crassa | 425 | DD | S10841 | | | | |
| I-NolI | Nol1931m | Nephroselmis | olivacea | 157 | D | AF110138 | | | | |
| I-NolII | Nol2593m | Nephroselmis | olivacea | 164 | D | AF110138 | | | | |
| I-PakI | | Pseudendoclonium | akinetum | 168 | D | AAL34378 | L44125 | | 2001 | Lucas et al. NAR Feb 15; 29(4): 960-9 |
| I-PanI | | Podospora | anserina | 243 | dd | X55026 | | | 1985 | Cummings, et al. |
| I-PcrI | Pcr1931c | Pterosperma | cristatum | 141 | D | L43359 | | | 2001 | Lucas et al. NAR Feb 15; 29(4): 960-9 |
| I-PtuI | Ptu1931c | Pedinomonas | tuberculata | 145 | DD | L43541 | | | 2001 | Lucas et al. NAR Feb 15; 29(4): 960-9 |
| I-ScaI | I-Sce VII | Saccharomyces | capensis | 243 | DD | X95974 | | | 2000 | Monteilhet, et al. J Mol Biol 185: 659-80 |
| I-SceI | | Saccharomyces | cerevisiae | 235 | dd | V00684 | | | 1985 | Jacquier & Dujon Nucleic Acids Res. 28: 1245-1251 |
| I-SceII | | Saccharomyces | cerevisiae | 316 | DD | P03878 | | | 1980 | Bonitz et al. J Biol Chem 255: 11927-41 |
| | | | | 315 | | | | | | |
| I-SceIII | | Saccharomyces | cerevisiae | 335 | DD | P03877 | | | 1980 | Bonitz et al. J Biol Chem 255: 11927-41 |
| I-SceIV | | Saccharomyces | cerevisiae | 307 | dd | S78650 | | | 1992 | Séraphin et al. Gene 113: 1-8 |
| I-SduI | Sdu2593c | Scherffelia | dubia | 167 | D | L44126 | | | 2001 | Lucas et al. NAR Feb 15; 29(4): 960-9 |
| I-SexI | I-Sex IP | Saccharomyces | exiguus | | D | | | | 2000 | http://rebase.neb.com/rebase/rebase.homing.html |
| | | (Candida holmii) | | | | | | | | |
| I-SneI | I-Sne IP, Sne1931b | Simkania | negevensis | 143 | D | AAD38228 | U68460 | | 1997 | Everett, et al. Int. J. Syst. Bacteriol. 47 (2), 461-473 |
| I-SobI | | Scenedesmus | obliquus | 221 | D | L43360 | | | 2001 | Lucas et al. NAR Feb 15; 29(4): 960-9 |
| I-SobII | Sob2593c | Scenedesmus | obliquus | 167 | D | L43360 | | | 2001 | Lucas et al. NAR Feb 15; 29(4): 960-9 |
| I-TmuI | Tmu2593c | Trichosarcina | mucosa | 168 | D | AAG61152 | AY008341 | | 2001 | Lucas et al. NAR Feb 15; 29(4): 960-9 |
| | ORF168 | | | | | | | | | |
| PI-AaeI | Aae RIR2 | Aquifex | aeolicus | 347 | DD | | | AE000657 | 1998 | Deckert, G. et al. Nature 392 (6674), 353-358 |
| PI-ApeI | Ape hyp3 | Aeropyrum | pernix | 468 | DD | B72665 | | | 1999 | Kawarabayasi, Y. et al DNA Res. 6 (2), 83-101 |
| PI-BspI | Bsp RIR1 | Bacteriophage | prophage A | 385 | DD | | | | 2001 | http://bioinformatics.weizmann.ac.il/~pietro/inteins |
| PI-CeuI | Ceu clpP | Chlamydomonas | eugametos | 457 | DD | | | | 2001 | http://bioinformatics.weizmann.ac.il/~pietro/inteins |
| PI-CirI | CIV RIR1 | Virus | | 339 | DD | | | NC_003038 | 2001 | http://bioinformatics.weizmann.ac.il/~pietro/inteins |
| PI-CtrI | PI-VDE II, Ctr VMA, | Candida | tropicalis | 471 | DD | M64984 | | | 1993 | Gu, et al. J Biol Chem 268: 7372-81 |
| | PI-SceII | | | | | | | | | |
| PI-DraI | Dra RIR1 | Deinococcus | radiodurans | 367 | DD | | | | 2001 | http://bioinformatics.weizmann.ac.il/~pietro/inteins |
| PI-DraII | Dra snf2 | Deinococcus | radiodurans | 343 | DD | | | | | |
| PI-MavI | Mav dnaB | Mycobacterium | avium | 337 | DD | | | | 2001 | http://bioinformatics.weizmann.ac.il/~pietro/inteins |
| PI-MchI | Mch recA | Mycobacterium | chitae | 365 | DD | | | | 2001 | http://bioinformatics.weizmann.ac.il/~pietro/inteins |
| PI-MfaI | Mfa recA | Mycobacterium | fallax | 364 | DD | | | | 2001 | http://bioinformatics.weizmann.ac.il/~pietro/inteins |
| PI-MflI | Mfl gyrA | Mycobacterium | flavescens | 421 | DD | | | | 2001 | http://bioinformatics.weizmann.ac.il/~pietro/inteins |
| PI-MflII | Mfl recA | Mycobacterium | flavescens | 364 | DD | | | | 2001 | http://bioinformatics.weizmann.ac.il/~pietro/inteins |
| PI-MflII 144 | Mfl recA 14474 | Mycobacterium | flavescens | 365 | DD | | | | 2001 | http://bioinformatics.weizmann.ac.il/~pietro/inteins |
| PI-MgaI | Mga gyrA | Mycobacterium | gastri | 420 | DD | | | | 2001 | http://bioinformatics.weizmann.ac.il/~pietro/inteins |
| PI-MgaII | Mga pps1 | Mycobacterium | gastri | 378 | DD | | | | 2001 | http://bioinformatics.weizmann.ac.il/~pietro/inteins |
| PI-MgaIII | Mga recA | Mycobacterium | gastri | 369 | DD | | | | 2001 | http://bioinformatics.weizmann.ac.il/~pietro/inteins |
| PI-MgoI | Mgo gyrA | Mycobacterium | gordonae | 420 | DD | | | | 2001 | http://bioinformatics.weizmann.ac.il/~pietro/inteins |
| PI-MinI | Min dnaB | Mycobacterium | intracellulare | 335 | DD | | | | 2001 | http://bioinformatics.weizmann.ac.il/~pietro/inteins |
| PI-MjaI | Mja GF6P | Methanococcus | jannaschii | 500 | DD | | | | 2001 | http://bioinformatics.weizmann.ac.il/~pietro/inteins |
| PI-MjaII | Mja helicase | Methanococcus | jannaschii | 502 | DD | | | | 2001 | http://bioinformatics.weizmann.ac.il/~pietro/inteins |
| PI-MjaIII | Mja hyp1 | Methanococcus | jannaschii | 393 | DD | | | | 2001 | http://bioinformatics.weizmann.ac.il/~pietro/inteins |
| PI-MjaIV | Mja IF2 | Methanococcus | jannaschii | 547 | DD | | | | 2001 | http://bioinformatics.weizmann.ac.il/~pietro/inteins |
| PI-MjaIX | Mja r-gyr | Methanococcus | jannaschii | 494 | DD | | | | 2001 | http://bioinformatics.weizmann.ac.il/~pietro/inteins |
| PI-MjaVI | Mja PEPSyn | Methanococcus | jannaschii | 413 | DD | | | | 2001 | http://bioinformatics.weizmann.ac.il/~pietro/inteins |
| PI-MjaVII | Mja pol-1 | Methanococcus | jannaschii | 369 | DD | | | | 2001 | http://bioinformatics.weizmann.ac.il/~pietro/inteins |
| PI-MjaVIII | Mja pol-2 | Methanococcus | jannaschii | 476 | DD | | | | 2001 | http://bioinformatics.weizmann.ac.il/~pietro/inteins |
| PI-MjaX | Mja RFC-1 | Methanococcus | jannaschii | 549 | DD | | | | 2001 | http://bioinformatics.weizmann.ac.il/~pietro/inteins |
| PI-MjaXI | Mja RFC-2 | Methanococcus | jannaschii | 437 | DD | | | | 2001 | http://bioinformatics.weizmann.ac.il/~pietro/inteins |
| PI-MjaXII | Mja RFC-3 | Methanococcus | jannaschii | 544 | DD | | | | 2001 | http://bioinformatics.weizmann.ac.il/~pietro/inteins |
| PI-MjaXIII | Mja RNR-1 | Methanococcus | jannaschii | 454 | DD | | | | 2001 | http://bioinformatics.weizmann.ac.il/~pietro/inteins |
| PI-MjaXIV | Mja RNR-2 | Methanococcus | jannaschii | 534 | DD | | | | 2001 | http://bioinformatics.weizmann.ac.il/~pietro/inteins |
| PI-MjaXIX | Mja UDPGD | Methanococcus | jannaschii | 455 | DD | | | | 2001 | http://bioinformatics.weizmann.ac.il/~pietro/inteins |
| PI-MjaXV | Mja RpolA″ | Methanococcus | jannaschii | 472 | DD | | | | 2001 | http://bioinformatics.weizmann.ac.il/~pietro/inteins |
| PI-MjaXVI | Mja RpolA′ | Methanococcus | jannaschii | 453 | DD | | | | 2001 | http://bioinformatics.weizmann.ac.il/~pietro/inteins |
| PI-MjaXVII | Mja rtcB4 | Methanococcus | jannaschii | 489 | DD | | | | 2001 | http://bioinformatics.weizmann.ac.il/~pietro/inteins |
| PI-MjaXVIII | Mja TFIIB | Methanococcus | jannaschii | 336 | DD | | | | 2001 | http://bioinformatics.weizmann.ac.il/~pietro/inteins |
| PI-MkaI | Mka gyrA | Mycobacterium | kansasii | 420 | DD | | | | 2001 | http://bioinformatics.weizmann.ac.il/~pietro/inteins |
| PI-MleI | Mle recA | Mycobacterium | leprae | 365 | Dd | X73822 | | | 1994 | Davis et al. EMBO J. Feb 1; 13(3): 699-703. |
| | | | | (366) | | | | | | |
| PI-MelII | Mle gyrA | Mycobacterium | leprae | 420 | DD | | | | 2001 | http://bioinformatics.weizmann.ac.il/~pietro/inteins |
| PI-MleIV | Mle pps1 | Mycobacterium | leprae | 387 | DD | | | | 2001 | http://bioinformatics.weizmann.ac.il/~pietro/inteins |
| PI-MmaI | Mma gyrA | Mycobacterium | malmoense | 420 | DD | | | | 2001 | http://bioinformatics.weizmann.ac.il/~pietro/inteins |
| PI-MshI | Msh recA | Mycobacterium | shimoidei | 365 | DD | | | | 2001 | http://bioinformatics.weizmann.ac.il/~pietro/inteins |
| PI-MsmII | Msm dnaB-2 | Mycobacterium | smegmatis | 426 | DD | | | | 2001 | http://bioinformatics.weizmann.ac.il/~pietro/inteins |
| PI-MthI | Mth recA | Mycobacterium | thermoresistibile | 366 | DD | | | | 2001 | http://bioinformatics.weizmann.ac.il/~pietro/inteins |
| PI-MtuHI | PI-MtuH IP, | Mycobacterium | tuberculosis | 415 | DD | | | | 2000 | http://rebase.neb.com/rebase/rebase.homing.html |
| | Mtu dnaB | | | | | | | | | |
| PI-MtuHII | PI-MtuH IIP, | Mycobacterium | tuberculosis | 360 | DD | | | | 2001 | http://bioinformatics.weizmann.ac.il/~pietro/inteins |
| | Mtu pps1 | | | | | | | | | |
| PI-MtuI | Mtu recA | Mycobacterium | tuberculosis | 439 | DD | X58485 | | | 1992 | Davis et al. Cell Oct 16; 71(2): 201-10 |
| | | | | (441) | | | | | | |
| PI-PabIII | Pab RIR1-1 | Pyrococcus | abyssi | 399 | DD | | | | 2001 | http://bioinformatics.weizmann.ac.il/~pietro/inteins |
| PI-PabIX | Pab RFC-2 | Pyrococcus | abyssi | 608 | DD | | | | 2001 | http://bioinformatics.weizmann.ac.il/~pietro/inteins |
| PI-PabV | Pab lon | Pyrococcus | abyssi | 333 | DD | | | | 2001 | http://bioinformatics.weizmann.ac.il/~pietro/inteins |
| PI-PabVI | Pab moaA | Pyrococcus | abyssi | 437 | DD | | | | 2001 | http://bioinformatics.weizmann.ac.il/~pietro/inteins |
| PI-PabVII | Pab IF2 | Pyrococcus | abyssi | 394 | DD | | | | 2001 | http://bioinformatics.weizmann.ac.il/~pietro/inteins |
| PI-PabVIII | Pab RFC-1 | Pyrococcus | abyssi | 499 | DD | | | | 2001 | http://bioinformatics.weizmann.ac.il/~pietro/inteins |
| PI-PabXI | Pab RIR1-2 | Pyrococcus | abyssi | 438 | DD | | | | 2001 | http://bioinformatics.weizmann.ac.il/~pietro/inteins |
| PI-PabXII | Pab RIR1-3 | Pyrococcus | abyssi | 382 | DD | | | | 2001 | http://bioinformatics.weizmann.ac.il/~pietro/inteins |
| PI-PabXIII | Pab rtcB4 | Pyrococcus | abyssi | 437 | DD | | | | 2001 | http://bioinformatics.weizmann.ac.il/~pietro/inteins |
| PI-PabXIV | Pab VMA | Pyrococcus | abyssi | 429 | DD | | | | 2001 | http://bioinformatics.weizmann.ac.il/~pietro/inteins |
| PI-PfuI | Pfu klbA | Pyrococcus | furiosus | 522 | DD | | | | 1999 | Komori, et al. NAR 27: 4167-74 |
| | | | | (525) | | | | | | |
| PI-PfuII | Pfu RIR1-2 | Pyrococcus | furiosus | 383 | DD | | | | 1999 | Komori, et al. NAR 27: 4167-74 |
| PI-PfuIII | Pfu RtcB, Pfu rtcB4 | Pyrococcus | furiosus | 481 | DD | | | | | |
| | (Pfu Hyp-2) | | | | | | | | | |
| PI-PfuIV | Pfu RIR1-1 | Pyrococcus | furiosus | 455 | DD | | | | | |
| PI-PfuIX | Pfu RFC | Pyrococcus | furiosus | 525 | DD | | | | | |
| PI-PfuV | Pfu IF2 | Pyrococcus | furiosus | 387 | DD | | | | | |
| PI-PfuVI | Pfu lon | Pyrococcus | furiosus | 401 | DD | | | | | |
| PI-PfuVII | Pfu CDC21 | Pyrococcus | furiosus | 367 | DD | | | | | |
| PI-PfuVIII | Pfu VMA | Pyrococcus | furiosus | 424 | DD | | | | | |
| PI-PfuX | Pfu topA | Pyrococcus | furiosus | 373 | DD | | | | | |
| PI-PhoI | Pho VMA | Pyrococcus | horikoshii | 377 | DD | | | | 2001 | http://bioinformatics.weizmann.ac.il/~pietro/inteins |
| PI-PhoIII | Pho pol | Pyrococcus | horikoshii | 460 | DD | | | | | |
| PI-PhoIX | Pho RIR1 | Pyrococcus | horikoshii | 385 | DD | | | | | |
| PI-PhoV | Pho IF2 | Pyrococcus | horikoshii | 445 | DD | | | | | |
| PI-PhoVI | Pho klbA | Pyrococcus | horikoshii | 521 | DD | | | | | |
| PI-PhoVII | Pho LHR | Pyrococcus | horikoshii | 476 | DD | | | | | |
| PI-PhoVIII | Pho RFC | Pyrococcus | horikoshii | 526 | DD | | | | | |
| PI-PhoX | Pho r-gyr | Pyrococcus | horikoshii | 410 | DD | | | | | |
| PI-PhoXIII | Pho lon | Pyrococcus | horikoshii | 475 | DD | | | | | |
| PI-PhoXIV | Pho RtcB, rtcB4 | Pyrococcus | horikoshii | 390 | DD | | | | 1997 | Takagi et al. Appl. Environ. Microbiol. 63: 4504-10 |
| | (Pho Hyp-2) | | | | | | | | | |
| PI-PkoI | Psp-KOD Pol-1 | Pyrococcus | kodakaraensis | 360 | DD | | | | | |
| | Pko pol-1 | | | | | | | | | |
| PI-PkoII | Pko Pol-2 | Pyrococcus | kodakaraensis | 537 | DD | | | | 1997 | Takagi et al. Appl. Environ. Microbial. 63: 4504-10 |
| | (Psp-KOD Pol-2) | | | | | | | | | |
| PI-PkoIII | Psp pol-3 | Pyrococcus | kodakaraensis | 537 | DD | | | | | |
| PI-PspI | Psp-GBD Pol, | Pyrococcus | species | 537 | DD | | | | 1993 | Xu, et al. Cell 75: 1371-77 |
| | Psp pol-1 | | | | | | | | | |
| PI-RmaI | PI-Rma43812 IP, | Rhodothermus | marinus | 428 | DD | | | | 1997 | Liu, et al. PNAS 94: 7851-56 |
| | Rma dnaB | | | | | | | | | |
| PI-SceI | PI-VDE I | Saccharomyces | cerevisiae | 454 | DD | M21609 | | | 1990 | Hirata, R. et al. J Biol Chem 265: 6726-6733 |
| PI-SPβI | PI-SPBeta IP | Bacteriophage | SPβ | 385 | DD | | | | 1998 | Lazarevic, et al. PNAS 95: 1692-97 |
| PI-SPβII | Spb RIR1 | Bacteriophage | SPβ | 385 | DD | | | | | |
| PI-SspI | Ssp dnaB | Synechocystis | species | 429 | DD | | | BA000022 | 2001 | http://bioinformatics.weizmann.ac.il/~pietro/inteins |
| PI-TagI | Tag pol-I | Thermococcus | aggregans | 360 | DD | | | | 2001 | http://bioinformatics.weizmann.ac.il/~pietro/inteins |
| | | (Pyrococcus) | | | | | | | | |
| PI-TagII | Tag pol-2 | Thermococcus | aggregans | 538 | DD | | | | 2001 | http://bioinformatics.weizmann.ac.il/~pietro/inteins |
| | | (Pyrococcus) | | | | | | | | |
| PI-TfuI | Tfu pol-1 | Thermococcus | fumicolans | 360 | DD | Z69882 | | | 2000 | Saves, et al. JBC 275: 2335-41 |
| | | (Pyrococcus) | | | | | | | | |
| PI-TfuII | Tfu pol-2 | Thermococcus | fumicolans | 389 | DD | Z69882 | | | 2000 | Saves, et al. JBC 275: 2335-41 |
| | | (Pyrococcus) | | | | | | | | |
| PI-ThyI | Thy pol-1 | Thermococcus | hydrothermalis | 538 | DD | | | | | |
| | | (Pyrococcus) | | | | | | | | |
| PI-ThyII | Thy pol-2 | Thermococcus | hydrothermalis | 390 | DD | | | | 2001 | http://bioinformatics.weizmann.ac.il/~pietro/inteins |
| | | (Pyrococcus) | | | | | | | | |
| PI-TliI | Tli pol-2, pI-TliIR | Thermococcus | litoralis | 390 | DD | M74198 | | | 1992 | Perler et al. PNAS 89: 5577-5581 |
| | | (Pyrococcus) | | | | | | | | |
| PI-TliII | Tli pol-1 | Thermococcus | litoralis | 541 | DD | M74198 | | | 1993 | Lambowitz, et al. Annu Rev Biochem 62: 587-622 |
| | | (Pyrococcus) | | | | | | | | |
| PI-TspI | Tsp-TY Pol-1 | Thermococcus | species | | DD | | | | | |
| | | (Pyrococcus) | | | | | | | | |
| PI-TspII | Tsp-TY Pol-2 | Thermococcus | species | 536 | DD | | | | 2001 | http://bioinformatics.weizmann.ac.il/~pietro/inteins |
| | | (Pyrococcus) | | | | | | | | |
| PI-TspIII | Tsp-TY Pol-3 | Thermococcus | species | 390 | DD | | | | 2001 | http://bioinformatics.weizmann.ac.il/~pietro/inteins |
| | | (Pyrococcus) | | | | | | | | |
| PI-TspIV | TspGE8 pol-1 | Thermococcus | species | 536 | DD | | | | | |
| | | (Pyrococcus) | | | | | | | | |
| PI-TspV | TspGE8 pol-2 | Thermococcus | species | 390 | DD | | | | | |
| | | (Pyrococcus) | | | | | | | | |

What is claimed:

1- A single-chain meganuclease comprising a first and a second domain in the orientation N-terminal toward C-terminal, wherein said first and second domains are derived from the same mono-dodecapeptide meganuclease and wherein said single-chain meganuclease is capable of causing DNA cleavage.

2- Single-chain meganuclease according to claim 1, wherein each of said first and second domains comprises a polypeptide fragment that is derived from said mono-dodecapeptide meganuclease, and wherein said polypeptide fragment comprises at least a first alpha-helix comprising a dodecapeptide motif flanked downstream by a DNA binding moiety comprising a 4-stranded beta-sheet flanked downstream by a further alpha-helix.

3- Single-chain meganuclease according to claim 1, wherein each of said first and second domains comprises a monomer of said mono-dodecapeptide meganuclease.

4- Single-chain meganuclease according to claim 1, wherein said first and second domains are joined by a linker.

5- Single-chain meganuclease according to claim 1, wherein said mono-dodecapeptide meganuclease is selected from the group consisting of the meganucleases comprising a “D” motif listed in the Table 4.

6- Single-chain meganuclease according to claim 1, wherein:
(a) each of said first and second domains that is derived from a parent I-CreI monomer, comprises a portion of said parent I-CreI monomer which extends at least from the beginning of the first alpha helix (α₁) to the end of the C-terminal loop of I-CreI and includes successively: the α₁β₁β₂α₂β₃β₄α₃ core domain, the α₄ and α₅ helices and the C-terminal loop of I-CreI, and
(b) the first and second domains are joined by a peptidic linker which allows said two domains to fold as a I-CreI dimer that is able to bind and cleave a chimeric DNA target comprising one different half of each parent homodimeric I-CreI meganuclease target sequence.

7- Single-chain meganuclease according to claim 6, wherein the first domain starts at position 1 or 6 of I-CreI and the second domain starts at position 2 or 6 of I-CreI.

8- Single-chain meganuclease according to claim 6, wherein the first and/or the second domain terminate(s) at position 145 of I-CreI.

9- Single-chain meganuclease according to claim 6, wherein the first and/or the second domain further include(s) at least the alpha 6 helix of I-CreI.

10- Single-chain meganuclease according to claim 6, wherein the first and/or the second domain terminate(s) at position 152, 156, 160 or 163 of I-CreI.

11- Single-chain meganuclease according to claim 6, wherein the peptidic linker consists of a sequence of 15 to 35 amino acids.

12- Single-chain meganuclease according to claim 6, wherein the peptidic linker is selected from the group consisting of the sequences 18 to 28 and 30 to 35.

13- Single-chain meganuclease according to claim 4, wherein said linker comprises a loop derived from a di-dodecapeptide meganuclease.

14- Single-chain meganuclease according to claim 4, wherein said linker comprises a loop derived from the I-DmoI di-dodecapeptide meganuclease.

15- Single-chain meganuclease according to claim 4, wherein said linker is a flexible polypeptide linker comprising glycine, serine and threonine residues.

16- Single-chain meganuclease according to claim 4, wherein said linker is selected from the group consisting of the sequences SEQ ID NO: 8 to 11.

17- Single-chain meganuclease according to claim 6, wherein both domains comprise different mutations at positions 26 to 40 and/or 44 to 77 of I-CreI, said single-chain I-CreI meganuclease being able to cleave a non-palindromic DNA sequence, wherein at least the nucleotides at positions +3 to +5, +8 to +10, −10 to −8 and −5 to −3 of said DNA sequence correspond to the nucleotides at positions +3 to +5, +8 to +10, −10 to −8 and −5 to −3 of a DNA target from a gene of interest.

18- Single-chain meganuclease according to claim 6, wherein at least one domain comprises a mutation at positions 137 to 143 of I-CreI that modifies the specificity of the single-chain I-CreI meganuclease towards the nucleotides at positions ±1 to 2, 6 to 7 and/or 11 to 12 of the I-CreI site.

19- Single-chain meganuclease according to claim 17, wherein said mutations are replacement of the initial amino acids with amino acids selected from the group consisting of: A, D, E, G, H, K, N, P, Q, R, S, T, Y, C, V, L and W.

20- Single-chain meganuclease according to claim 6, which comprises mutation(s) that impair(s) the formation of functional homodimers from the two domains.

21- Single-chain meganuclease according to claim 6, wherein each domain comprises at least one mutation, selected from the group consisting of: K7E or K7D and E8K or E8R; F54G or F54A and L97F or L97W; K96D or K96E and E61R or E61K; R51D or R51E and D137R or D137K, respectively for the first and the second domain.

22- Single-chain meganuclease according to claim 6, wherein one domain comprises the substitution of the lysine residues at positions 7 and 96 by an acidic amino acid and the other domain comprises the substitution of the glutamic acid residues at positions 8 and 61 by a basic amino acid.

23- Single-chain meganuclease according to claim 6, wherein one domain comprises the G19S mutation.

24- Single-chain meganuclease according to claim 23, wherein the other domain or both domains comprise(s) at least one mutation that impairs the formation of a functional homodimer.

25- Single-chain meganuclease according to claim 1, which comprises a sequence selected from the group consisting of the sequences SEQ ID NO: 6, 111 and 113.

26- A purified or recombinant polynucleotide encoding a meganuclease according to claim 1.

27- A purified or recombinant polynucleotide comprising a hybrid target and cleavage site for a meganuclease according to claim 1.

28- Polynucleotide according to claim 27, wherein said site comprises two half-sites of the initial mono-dodecapeptide meganuclease.

29- A vector comprising a polynucleotide according to claim 26.

30- A host cell comprising a polynucleotide according to claim 26.

31- A method of genetic engineering comprising the steps of:
introducing a double-strand break at a targeting locus comprising a hybrid target site with the corresponding meganuclease according to claim 1; and
providing a targeting construct comprising the sequence to introduce flanked by homologous sequence to the targeting locus.

32- Method according to claim 31, wherein said targeting locus is a genomic locus.

33- Method according to claim 31, wherein said meganuclease is provided either by an expression vector comprising a polynucleotide according to claim 26 or by said meganuclease itself.

34- A method of deleting a viral genome or a part thereof, wherein a double-strand break in the viral genome is induced by a meganuclease according to claim 1 and said double-strand break induces a recombination event leading to the deletion of the viral genome or a part thereof.


SEQUENCE LISTING (129 sequences)

SEQ ID NO: 1 - DNA, 795 bp, Artificial Sequence
  Description of Artificial Sequence Synthetic polynucleotide
atggcccataacaatgagaacgtttctggtatctccgcttacctgctgggcctgattatcggtgatggtggcctgtacaagctgaaatataaaggtaaccgtagcgaatatcgtgttgtgatcacccagaagtctgaaaacctgattaaacaacacatcgcaccgctgatgcagtttctgattgatgaactgaatgtgaaatctaaaatccagatcgttaagggtgatacccgctatgagctgcgtgtgagctctaagaaactgtactattacttcgctaacatgctggagcgtatccgcctgttcaacatgcgtgagcagctggcgttcctggccggctttgtggacggtgacggtagcatcatcgctcagattaaaccaaaccagtcttataaattcaagcatcagctgtccctgacctttcaggtgactcaaaagacccagcgccgttggtttctggacaaactggtggatgaaattggcgttggttacgtacgtgatcgcggtagcgtttccgattacattctgagcgaaatcaagccgctgcacaacttcctgactcaactgcaaccgtttctgaaactgaaacagaaacaggcaaacctggttctgaaaattatcgaacagctgccgtctgcaaaagaatccccggacaaattcctggaagtttgtacctgggtggatcagattgcagctctgaacgattctaagacgcgtaaaaccacttctgaaaccgttcgtgctgtgctggacagcctgagcgagaagaagaaatcctccccggcggccgactag

SEQ ID NO: 2 - protein, 264 aa, Artificial Sequence
  Description of Artificial Sequence Synthetic polypeptide
MAHNNENVSGISAYLLGLIIGDGGLYKLKYKGNRSEYRVVITQKSENLIKQHIAPLMQFLIDELNVKSKIQIVKGDTRYELRVSSKKLYYYFANMLERIRLFNMREQLAFLAGFVDGDGSIIAQIKPNQSYKFKHQLSLTFQVTQKTQRRWFLDKLVDEIGVGYVRDRGSVSDYILSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 3 - DNA, 795 bp, Artificial Sequence
  Description of Artificial Sequence Synthetic polynucleotide
atggcccacaacaatgagaatgtgtctggcatctctgcctacctgctgggcctcatcattggagatggaggtctgtacaaacttaagtacaaaggcaacaggtctgagtacagagtggtcatcacccagaagtctgaaaatgctatcaagcaagctattgctccagatatgcagttcctgattgatgaactcaatgtcaagagcaagatccagattgtcaaaggtgacactagatatgagctgagagtttcctccaagaaactttactattactttgccaacatgttggagaggatcaggctgttcaatatgagggagcaactggccttccttgctggatttgtggatggtgatggctccatcattgctcagataaaaccaaatcaatcttacaagttcaaacaccagctctccttgacctttcaagtcactcagaagacacaaagaaggtggttcttggacaaattggttgatgagattggtgtgggctatgtcagagacagaggctctgtgtcagactacatcctgtctgaaattaagcctcttcataactttctcacccaactgcaacccttcttgaagctcaaacagaagcaagcaaatctggttttgaaaatcattgaacagctgccatctgccaaggagtcccctgacaagtttcttgaagtgtgtacttgggtggatcagattgctgccttgaatgactccaagaccagaaaaaccacctctgagactgtgagggcagttctggatagcctctctgagaagaaaaagtcctctcctgcggccgactag

SEQ ID NO: 4 - protein, 264 aa, Artificial Sequence
  Description of Artificial Sequence Synthetic polypeptide
MAHNNENVSGISAYLLGLIIGDGGLYKLKYKGNRSEYRVVITQKSENAIKQAIAPDMQFLIDELNVKSKIQIVKGDTRYELRVSSKKLYYYFANMLERIRLFNMREQLAFLAGFVDGDGSIIAQIKPNQSYKFKHQLSLTFQVTQKTQRRWFLDKLVDEIGVGYVRDRGSVSDYILSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 5 - DNA, 795 bp, Artificial Sequence
  Description of Artificial Sequence Synthetic polynucleotide
atggccaacactaagtacaataaagaatttctcctgtatctggcaggtttcgtcgacggcgatggctccattatcgcacagatcaagccgaatcagagctacaagtttaaacaccaactgtctctcactttccaggttacccagaaaactcaacgtcgctggttcctggataagctggtagatgagatcggtgtgggctatgtacgcgaccgtggctctgtgagcgactatatcctgtctgagattaaaccactgcataattttctgacccagctgcaggctatgctggagcgtatccgtctgttcaacatgcgtgagttcctgctgtacctggccggctttgtggacggtgacggtagcatcatcgctcagattaaaccaaaccagtcttataaattcaagcatcagctgtccctgacctttcaggtgactcaaaagacccagcgccgttggtttctggacaaactggtggatgaaattggcgttggttacgtacgtgatcgcggtagcgtttccgattacattctgagcgaaatcaagccgctgcacaacttcctgactcaactgcaaccgtttctgaaactgaaacagaaacaggcaaacctggttctgaaaattatcgaacagctgccgtctgcaaaagaatccccggacaaattcctggaagtttgtacctgggtggatcagattgcagctctgaacgattctaagacgcgtaaaaccacttctgaaaccgttcgtgctgtgctggacagcctgagcgagaagaagaaatcctccccggcggccgactag

SEQ ID NO: 6 - protein, 264 aa, Artificial Sequence
  Description of Artificial Sequence Synthetic polypeptide
MANTKYNKEFLLYLAGFVDGDGSIIAQIKPNQSYKFKHQLSLTFQVTQKTQRRWFLDKLVDEIGVGYVRDRGSVSDYILSEIKPLHNFLTQLQAMLERIRLFNMREFLLYLAGFVDGDGSIIAQIKPNQSYKFKHQLSLTFQVTQKTQRRWFLDKLVDEIGVGYVRDRGSVSDYILSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 7 - protein, 17 aa, Artificial Sequence
  Description of Artificial Sequence Synthetic peptide
NMLERIRLFNMREQLAF

SEQ ID NO: 8 - protein, 12 aa, Artificial Sequence
  Description of Artificial Sequence Synthetic peptide
AMLERIRLFNMR

SEQ ID NO: 9 - protein, 12 aa, Artificial Sequence
  Description of Artificial Sequence Synthetic peptide
AFLERIRLFNMR

SEQ ID NO: 10 - protein, 12 aa, Artificial Sequence
  Description of Artificial Sequence Synthetic peptide
AMLERIRLYNMR

SEQ ID NO: 11 - protein, 12 aa, Artificial Sequence
  Description of Artificial Sequence Synthetic peptide
AFLERIRLYNMR

SEQ ID NO: 12 - DNA, 27 bp, Artificial Sequence
  Description of Artificial Sequence Synthetic oligonucleotide
caaaacgtcgtgagacagtttggtcca

SEQ ID NO: 13 - DNA, 27 bp, Artificial Sequence
  Description of Artificial Sequence Synthetic oligonucleotide
ccttgccgggtaagttccggcgcgcat

SEQ ID NO: 14 - DNA, 27 bp, Artificial Sequence
  Description of Artificial Sequence Synthetic oligonucleotide
ccttgccgggtgagacagtttggtcca

SEQ ID NO: 15 - DNA, 27 bp, Artificial Sequence
  Description of Artificial Sequence Synthetic oligonucleotide
caaaacgtcgtaagttccggcgcgcat

SEQ ID NO: 16 - DNA, 18 bp, Artificial Sequence
  Description of Artificial Sequence Synthetic oligonucleotide
tagggataacagggtaat

SEQ ID NO: 17 - protein, 11 aa, Artificial Sequence
  Description of Artificial Sequence Synthetic peptide
MLERIRLFNMR

SEQ ID NO: 18 - protein, 32 aa, Artificial Sequence
  Description of Artificial Sequence Synthetic polypeptide
AAGGSDKYNQALSKYNQALSKYNQALSGGGGS

SEQ ID NO: 19 - protein, 22 aa, Artificial Sequence
  Description of Artificial Sequence Synthetic peptide
AAGGGGSGGGGSGGGGSGGGGS

SEQ ID NO: 20 - protein, 35 aa, Artificial Sequence
  Description of Artificial Sequence Synthetic polypeptide
AAGKSSDSKGIDLTNVTLPDTPTYSKAASDAIPPA

SEQ ID NO: 21 - protein, 31 aa, Artificial Sequence
  Description of Artificial Sequence Synthetic polypeptide
AAGLEYPQAPYSSPPGPPCCSGSSGSSAGCS

SEQ ID NO: 22 - protein, 30 aa, Artificial Sequence
  Description of Artificial Sequence Synthetic polypeptide
AAGLSYHYSNGGSPTSDGPALGGISDGGAT

SEQ ID NO: 23 - protein, 27 aa, Artificial Sequence
  Description of Artificial Sequence Synthetic peptide
AAGDSSVSNSEHIAPLSLPSSPPSVGS

SEQ ID NO: 24 - protein, 25 aa, Artificial Sequence
  Description of Artificial Sequence Synthetic peptide
AAGASQGCKPLALPELLTEDSYNTD

SEQ ID NO: 25 - protein, 24 aa, Artificial Sequence
  Description of Artificial Sequence Synthetic peptide
AAGNPIPGLDELGVGNSDAAAPGT

SEQ ID NO: 26 - protein, 23 aa, Artificial Sequence
  Description of Artificial Sequence Synthetic peptide
AAGAPTECSPSALTQPPSASGSL

SEQ ID NO: 27 - protein, 27 aa, Artificial Sequence
  Description of Artificial Sequence Synthetic peptide
AAGGGGSGGGGSGGGGSGGGGSGGGGS

SEQ ID NO: 28 - protein, 17 aa, Artificial Sequence
  Description of Artificial Sequence Synthetic peptide
AAGGGGSGGGGSGGGGS

SEQ ID NO: 29 - protein, 12 aa, Artificial Sequence
  Description of Artificial Sequence Synthetic peptide
AAGGGGSGGGGS

SEQ ID NO: 30 - protein, 17 aa, Artificial Sequence
  Description of Artificial Sequence Synthetic peptide
AAGQVTSAAGPATVPSG

SEQ ID NO: 31 - protein, 19 aa, Artificial Sequence
  Description of Artificial Sequence Synthetic peptide
AAGGSPLKPSAPKIPIGGS

SEQ ID NO: 32 - protein, 33 aa, Artificial Sequence
  Description of Artificial Sequence Synthetic polypeptide
AAGGSPLKPSAPKIPIGGSPLKPSAPKIPIGGS

SEQ ID NO: 33 - protein, 15 aa, Artificial Sequence
  Description of Artificial Sequence Synthetic peptide
AAGGSPLSKPIPGGS

SEQ ID NO: 34 - protein, 25 aa, Artificial Sequence
  Description of Artificial Sequence Synthetic peptide
AAGGSPLSKPIPGGSPLSKPIPGGS

SEQ ID NO: 35 - protein, 31 aa, Artificial Sequence
  Description of Artificial Sequence Synthetic polypeptide
AAGGSDKYNQALSERRAYVVANNLVSGGGGS

SEQ ID NO: 36 - protein, 163 aa, Chlamydomonas reinhardtii
MNTKYNKEFLLYLAGFVDGDGSIIAQIKPNQSYKFKHQLSLTFQVTQKTQRRWFLDKLVDEIGVGYVRDRGSVSDYILSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 37 - DNA, 22 bp, Artificial Sequence
  Description of Artificial Sequence Synthetic oligonucleotide
caaaacgtcgtacgacgttttg

SEQ ID NO: 38 - DNA, 501 bp, Artificial Sequence
  Description of Artificial Sequence Synthetic polynucleotide
atggccaataccaaatataacaaagagttcctgctgtacctggccggctttgtggacggtgacggtagcatcatcgctcagattaaaccaaaccagtcttataagtttaaacatcagctaagcttgacctttcaggtgactcaaaagacccagcgccgttggtttctggacaaactagtggatgaaattggcgttggttacgtacgtgatcgcggatccgtttccaactacatcttaagcgaaatcaagccgctgcacaacttcctgactcaactgcagccgtttctgaaactgaaacagaaacaggcaaacctggttctgaaaattatcgaacagctgccgtctgcaaaagaatccccggacaaattcctggaagtttgtacctgggtggatcagattgcagctctgaacgattctaagacgcgtaaaaccacttctgaaaccgttcgtgctgtgctggacagcctgagcgagaagaagaaatcctccccggcggccgac

SEQ ID NO: 39 - protein, 167 aa, Artificial Sequence
  Description of Artificial Sequence Synthetic polypeptide
MANTKYNKEFLLYLAGFVDGDGSIIAQIKPNQSYKFKHQLSLTFQVTQKTQRRWFLDKLVDEIGVGYVRDRGSVSNYILSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 40 - DNA, 492 bp, Artificial Sequence
  Description of Artificial Sequence Synthetic polynucleotide
atgaacaccaagtacaacaaagagttcctgctgtatcttgctggatttgtggatggtgatggctccatcattgctcagataaaaccaaatcaatcttacaagttcaaacaccagctctccttggcctttcaagtcactcagaagacacaaagaaggtggttcttggacaaattggttgatgagattggtgtgggctatgtcagagacagaggctctgtgtcagactacatcctgtctgaaattaagcctcttcataactttctcacccaactgcaacccttcttgaagctcaaacagaagcaagcaaatctggttttgaaaatcatctggagactgccatctgccaaggagtcccctgacaagtttcttgaagtgtgtacttgggtggatcagattgctgccttgaatgactccaagaccagaaaaaccacctctgagactgtgagggcagttctggatagcctctctgagaagaaaaagtcctctccttag

SEQ ID NO: 41 - protein, 163 aa, Artificial Sequence
  Description of Artificial Sequence Synthetic polypeptide
MNTKYNKEFLLYLAGFVDGDGSIIAQIKPNQSYKFKHQLSLAFQVTQKTQRRWFLDKLVDEIGVGYVRDRGSVSDYILSEIKPLHNFLTQLQPFLKLKQKQANLVLKIIWRLPSAKESPDKFLEVCTWVDQIAALNDSKTRKTTSETVRAVLDSLSEKKKSSP

SEQ ID NO: 42 - DNA, 22 bp, Artificial Sequence
  Description of Artificial Sequence Synthetic oligonucleotide
cgagatgtcacacagaggtacg

SEQ ID NO: 43 - DNA, 22 bp, Artificial Sequence
  Description of Artificial Sequence Synthetic oligonucleotide
cgagacgtcgtacgacgtctcg

SEQ ID NO: 44 - DNA, 22 bp, Artificial Sequence
  Description of Artificial Sequence Synthetic oligonucleotide
cgtaacgtcgtacgacgttacg

SEQ ID NO: 45 - DNA, 22 bp, Artificial Sequence
  Description of Artificial Sequence Synthetic oligonucleotide
caaaactctgtacagagttttg

SEQ ID NO: 46 - DNA, 22 bp, Artificial Sequence
  Description of Artificial Sequence Synthetic oligonucleotide
cgagatgtcgtacgacatctcg

SEQ ID NO: 47 - DNA, 22 bp, Artificial Sequence
  Description of Artificial Sequence Synthetic oligonucleotide
cgtacctctgtacagaggtacg

SEQ ID NO: 48 - DNA, 22 bp, Artificial Sequence
  Description of Artificial Sequence Synthetic oligonucleotide
ccatacgtcgtacgacgtatgg

SEQ ID NO: 49 - DNA, 22 bp, Artificial Sequence
  Description of Artificial Sequence Synthetic oligonucleotide
caaaaccttgtacaaggttttg

SEQ ID NO: 50 - DNA, 22 bp, Artificial Sequence
  Description of Artificial Sequence Synthetic oligonucleotide
cgagatgtcatgaaagagatgg

SEQ ID NO: 51 - DNA, 22 bp, Artificial Sequence
  Description of Artificial Sequence Synthetic oligonucleotide
cgagatgtcgtacgacatctcg

SEQ ID NO: 52 - DNA, 22 bp, Artificial Sequence
  Description of Artificial Sequence Synthetic oligonucleotide
ccatctcttgtacaagagatgg

SEQ ID NO: 53 - DNA, 22 bp, Artificial Sequence
  Description of Artificial Sequence Synthetic oligonucleotide
cgttacgtcgtacgacgtaacg

SEQ ID NO: 54 - DNA, 22 bp, Artificial Sequence
  Description of Artificial Sequence Synthetic oligonucleotide
caaaaccaggtacctggttttg

SEQ ID NO: 55 - DNA, 22 bp, Artificial Sequence
  Description of Artificial Sequence Synthetic oligonucleotide
ctggacgtcgtacgacgtccag

SEQ ID NO: 56 - DNA, 22 bp, Artificial Sequence
  Description of Artificial Sequence Synthetic oligonucleotide
caaaacgaggtacctcgttttg

SEQ ID NO: 57 - DNA, 22 bp, Artificial Sequence
  Description of Artificial Sequence Synthetic oligonucleotide
tgttctcaggtacctcagccag

SEQ ID NO: 58 - DNA, 22 bp, Artificial Sequence
  Description of Artificial Sequence Synthetic oligonucleotide
tgttctcaggtacctgagaaca

SEQ ID NO: 59 - DNA, 22 bp, Artificial Sequence
  Description of Artificial Sequence Synthetic oligonucleotide
ctggctgaggtacctcagccag

SEQ ID NO: 60 - DNA, 26 bp, Artificial Sequence
  Description of Artificial Sequence Synthetic primer
gcaactttagtgctgacacatacagg

SEQ ID NO: 61 - DNA, 33 bp, Artificial Sequence
  Description of Artificial Sequence Synthetic primer
ctggtgtttgaacttgtgagattgatttggttt

SEQ ID NO: 62 - DNA, 33 bp, Artificial Sequence
  Description of Artificial Sequence Synthetic primer
aaaccaaatcaatctcacaagttcaaacaccag

SEQ ID NO: 63 - DNA, 24 bp, Artificial Sequence
  Description of Artificial Sequence Synthetic primer
acaaccttgattggagacttgacc

SEQ ID NO: 64 - DNA, 37 bp, Artificial Sequence
  Description of Artificial Sequence Synthetic primer
tagacgagctcctaaggagaggactttttcttctcag

SEQ ID NO: 65 - DNA, 98 bp, Artificial Sequence
  Description of Artificial Sequence Synthetic primer
tatcggccggtggcggaggatctggcggcggtggatccggtggtggaggctccggaggaggtggctctaacaaagagttcctgctgtatcttgctgga

SEQ ID NO: 66 - DNA, 140 bp, Artificial Sequence
  Description of Artificial Sequence Synthetic primer
tatcggccggtaaatcttccgattccaagggtattgatctgactaatgttactctgcctgatacccctacttattccaaagctgcctctgatgctattcctccagctaacaaagagttcctgctgtatcttgctggattt

SEQ ID NO: 67 - DNA, 125 bp, Artificial Sequence
  Description of Artificial Sequence Synthetic primer
tatcggccggtctggagtatcaggctccttactcttcccctccaggtcctccttgttgctccggttcctctggctcctctgctggttgttctaacaaagagttcctgctgtatcttgctggattt

SEQ ID NO: 68 - DNA, 125 bp, Artificial Sequence
  Description of Artificial Sequence Synthetic primer
tatcggccggtctgtcctatcattattctaatggtggctcccctacttctgatggtccagctctgggtggcatttctgatggtggcgctactaacaaagagttcctgctgtatcttgctggattt

SEQ ID NO: 69 - DNA, 116 bp, Artificial Sequence
  Description of Artificial Sequence Synthetic primer
tatcggccggtgattcctctgtttctaattccgagcacattgctcctctgtctctgccttcctctcctccatctgttggttctaacaaagagttcctgctgtatcttgctggattt

SEQ ID NO: 70 - DNA, 110 bp, Artificial Sequence
  Description of Artificial Sequence Synthetic primer
tatcggccggtgcttctcagggttgtaaacctctggctctgcctgagctgcttactgaggattcttataatactgataacaaagagttcctgctgtatcttgctggattt

SEQ ID NO: 71 - DNA, 107 bp, Artificial Sequence
  Description of Artificial Sequence Synthetic primer
tatcggccggtaatcctattcctggtctggatgagctgggtgttggcaactctgatgctgccgctcctggcactaacaaagagttcctgctgtatcttgctggattt

SEQ ID NO: 72 - DNA, 104 bp, Artificial Sequence
  Description of Artificial Sequence Synthetic primer
tatcggccggtgctcctactgagtgttctccttccgctctgacccagcctccatccgcttctggttccctgaacaaagagttcctgctgtatcttgctggattt

SEQ ID NO: 73 - DNA, 116 bp, Artificial Sequence
  Description of Artificial Sequence Synthetic primer
tatcggccggtggaggcggttctggaggcggtggctctggtggaggcggttccggtggaggcggatctggtggaggcggttctaacaaagagttcctgctgtatcttgctggattt

SEQ ID NO: 74 - DNA, 86 bp, Artificial Sequence
  Description of Artificial Sequence Synthetic primer
tatcggccggtggaggcggttctggaggcggtggctctggtggaggcggttccaacaaagagttcctgctgtatcttgctggattt

SEQ ID NO: 75 - DNA, 71 bp, Artificial Sequence
  Description of Artificial Sequence Synthetic primer
tatcggccggtggaggcggttctggaggcggtggctctaacaaagagttcctgctgtatcttgctggattt

SEQ ID NO: 76 - DNA, 86 bp, Artificial Sequence
  Description of Artificial Sequence Synthetic primer
tatcggccggtcaggttacttctgctgccggtcctgctactgttccatctggtaacaaagagttcctgctgtatcttgctggattt

SEQ ID NO: 77 - DNA, 92 bp, Artificial Sequence
  Description of Artificial Sequence Synthetic primer
tatcggccggtggatctcctctgaagccttctgccccaaagattcctataggtggctccaacaaagagttcctgctgtatcttgctggattt

SEQ ID NO: 78 - DNA, 134 bp, Artificial Sequence
  Description of Artificial Sequence Synthetic primer
tatcggccggtggatctcctctgaagccttctgccccaaagattcctataggtggctccccactgaaaccttccgcacctaaaatcccaattggtggctctaacaaagagttcctgctgtatcttgctggattt

SEQ ID NO: 79 - DNA, 80 bp, Artificial Sequence
  Description of Artificial Sequence Synthetic primer
tatcggccggtggatctcctctgtctaaaccaattccaggcggttccaacaaagagttcctgctgtatcttgctggattt

SEQ ID NO: 80 - DNA, 110 bp, Artificial Sequence
  Description of Artificial Sequence Synthetic primer
tatcggccggtggatctcctctgtctaaaccaattccaggcggttccccactgtcaaagccaatccctggcggttctaacaaagagttcctgctgtatcttgctggattt

SEQ ID NO: 81 - DNA, 128 bp, Artificial Sequence
  Description of Artificial Sequence Synthetic primer
tatcggccggtggatctgataagtataatcaggctctgtctgagcgtcgcgcctacgttgtcgccaataacctggtttccggtggaggcggttccaacaaagagttcctgctgtatcttgctggattt

SEQ ID NO: 82 - DNA, 131 bp, Artificial Sequence
  Description of Artificial Sequence Synthetic primer
tatcggccggtggatctgataagtataatcaggctctgtctaaatacaaccaagcactgtccaagtacaatcaggccctgtctggtggaggcggttccaacaaagagttcctgctgtatcttgctggattt

SEQ ID NO: 83 - DNA, 35 bp, Artificial Sequence
  Description of Artificial Sequence Synthetic primer
gcaatgatggagccatcagaatccacaaatccagc

SEQ ID NO: 84 - DNA, 35 bp, Artificial Sequence
  Description of Artificial Sequence Synthetic primer
gctggatttgtggattctgatggctccatcattgc

SEQ ID NO: 85 - DNA, 34 bp, Artificial Sequence
  Description of Artificial Sequence Synthetic primer
aataccaaatataacgaagagttcctgctgtacc

SEQ ID NO: 86 - DNA, 33 bp, Artificial Sequence
  Description of Artificial Sequence Synthetic primer
aagatacagcaggaactttttgttagagccacc

SEQ ID NO: 87 - DNA, 33 bp, Artificial Sequence
  Description of Artificial Sequence Synthetic primer
ggtggctctaacaaaaagttcctgctgtatctt

SEQ ID NO: 88 - DNA, 32 bp, Artificial Sequence
  Description of Artificial Sequence Synthetic primer
caggtacagcaggaactttttgttatatttgg

SEQ ID NO: 89 - DNA, 34 bp, Artificial Sequence
  Description of Artificial Sequence Synthetic primer
accaaatataacaaaaagttcctgctgtacctgg

SEQ ID NO: 90 - DNA, 33 bp, Artificial Sequence
  Description of Artificial Sequence Synthetic primer
aagatacagcaggaactcttcgttagagccacc

SEQ ID NO: 91 - DNA, 33 bp, Artificial Sequence
  Description of Artificial Sequence Synthetic primer
ggtggctctaacgaagagttcctgctgtatctt

SEQ ID NO: 92 - DNA, 84 bp, Artificial Sequence
  Description of Artificial Sequence Synthetic primer
aagcagagctctctggctaactagagaacccactgcttactggcttatcgaccatggccaataccaaatataacaaagagttcc

SEQ ID NO: 93 - DNA, 36 bp, Artificial Sequence
  Description of Artificial Sequence Synthetic primer
ctgctctagactaaggagaggactttttcttctcag

SEQ ID NO: 94 - DNA, 38 bp, Artificial Sequence
  Description of Artificial Sequence Synthetic primer
tagacgagctcctacggggaggatttcttcttctcgct

SEQ ID NO: 95 - DNA, 32 bp, Artificial Sequence
  Description of Artificial Sequence Synthetic primer
acaggccatggccaataccaaatataacaaag

SEQ ID NO: 96 - DNA, 37 bp, Artificial Sequence
  Description of Artificial Sequence Synthetic primer
acaggccatggccaacaaagagttcctgctgtacctg

SEQ ID NO: 97 - DNA, 31 bp, Artificial Sequence
  Description of Artificial Sequence Synthetic primer
gattgacggccgccggggaggatttcttctt

SEQ ID NO: 98 - DNA, 37 bp, Artificial Sequence
  Description of Artificial Sequence Synthetic primer
gattgacggccgctttcttcttctcgctcaggctgtc

SEQ ID NO: 99 - DNA, 37 bp, Artificial Sequence
  Description of Artificial Sequence Synthetic primer
gattgacggccgcgctcaggctgtccaggacagcacg

SEQ ID NO: 100 - DNA, 37 bp, Artificial Sequence
  Description of Artificial Sequence Synthetic primer
gattgacggccgccaggacagcacgaacggtttcaga

SEQ ID NO: 101 - DNA, 37 bp, Artificial Sequence
  Description of Artificial Sequence Synthetic primer
gattgacggccgcagaagtggttttacgcgtcttaga

SEQ ID NO: 102 - DNA, 131 bp, Artificial Sequence; identical to SEQ ID NO: 82
  Description of Artificial Sequence Synthetic primer

SEQ ID NO: 103 - DNA, 131 bp, Artificial Sequence
  Description of Artificial Sequence Synthetic primer
tatcggccggtggatctgataagtataatcaggctctgtctaaatacaaccaagcactgtccaagtacaatcaggccctgtctggtggaggcggttccaacaccaagtacaacaaagagttcctgctgtat

SEQ ID NO: 104 - DNA, 37 bp, Artificial Sequence; identical to SEQ ID NO: 64
  Description of Artificial Sequence Synthetic primer

SEQ ID NO: 105 - DNA, 38 bp, Artificial Sequence
  Description of Artificial Sequence Synthetic primer
tagacgagctcctactttttcttctcagagaggtcatc

SEQ ID NO: 106 - DNA, 38 bp, Artificial Sequence
  Description of Artificial Sequence Synthetic primer
tagacgagctcctaagagaggtcatccagaactgccct

SEQ ID NO: 107 - DNA, 38 bp, Artificial Sequence
  Description of Artificial Sequence Synthetic primer
tagacgagctcctacagaactgccctcacagtctcaga

SEQ ID NO: 108 - DNA, 38 bp, Artificial Sequence
  Description of Artificial Sequence Synthetic primer
tagacgagctcctaagaggtggtttttctggtcttgga

SEQ ID NO: 109 - DNA, 84 bp, Artificial Sequence
  Description of Artificial Sequence Synthetic primer
aagcagagctctctggctaactagagaacccactgcttactggcttatcgaccatggccaataccaaatataacaaaaagttcc

SEQ ID NO: 110 - DNA, 1065 bp, Artificial Sequence
  Description of Artificial Sequence Synthetic polynucleotide
atggccaataccaaatataacaaagagttcctgctgtacctggccggctttgtggacggtgacggtagcatcatcgctcagattaatccaaaccagtcttctaagtttaaacatcgtctacgtttgaccttttatgtgactcaaaagacccagcgccgttggtttctggacaaactagtggatgaaattggcgttggttacgtacgtgattctggatccgtttcccagtacgttttaagcgaaatcaagccgctgcacaacttcctgactcaactgcagccgtttctgaaactgaaacagaaacaggcaaacctggttctgaaaattatcgaacagctgccgtctgcaaaagaatccccggacaaattcctggaagtttgtacctgggtggatcagattgcagctctgaacgattctaagacgcgtaaaaccacttctgaaaccgttcgtgctgtgctggacagcctgagcgggaagaagaaatcctccccggcggccggtggatctgataagtataatcaggctctgtctaaatacaaccaagcactgtccaagtacaatcaggccctgtctggtggaggcggttccaacaaagagttcctgctgtatcttgctggatttgtggattctgatggctccatcattgctcagataaaaccacgtcaatctaacaagttcaaacaccagctctccttgacttttgcagtcactcagaagacacaaagaaggtggttcttggacaaattggttgatgagattggtgtgggctatgtctatgacagtggctctgtgtcagactaccgcctgtctgaaattaagcctcttcataactttctcacccaactgcaacccttcttgaagctcaaacagaagcaagcaaatctggttttgaaaatcatcgagcaactgccatctgccaaggagtcccctgacaagtttcttgaagtgtgtacttgggtggatcagattgctgccttgaatgactccaagaccagaaaaaccacctctgagactgtgagggcagttctggatagcctctctgagaagaaaaagtcctctccttag

SEQ ID NO: 111 - protein, 354 aa, Artificial Sequence
  Description of Artificial Sequence Synthetic polypeptide
MANTKYNKEFLLYLAGFVDGDGSIIAQINPNQSSKFKHRLRLTFYVTQKTQRRWFLDKLVDEIGVGYVRDSGSVSQYVLSEIKPLHNFLTQLQPFLKLKQKQANLVLKIIEQLPSAKESPDKFLEVCTWVDQIAALNDSKTRKTTSETVRAVLDSLSGKKKSSPAAGGSDKYNQALSKYNQALSKYNQALSGGGGSNKEFLLYLAGFVDSDGSIIAQIKPRQSNKFKHQLSLTFAVTQKTQRRWFLDKLVDEIGVGYVYDSGSVSDYRLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 112 - DNA, 1065 bp, Artificial Sequence
  Description of Artificial Sequence Synthetic polynucleotide
atggccaataccaaatataacgaagagttcctgctgtacctggccggctttgtggacggtgacggtagcatcatcgctcagattaatccaaaccagtcttctaagtttaaacatcgtctacgtttgaccttttatgtgactcaaaagacccagcgccgttggtttctggacaaactagtggatgaaattggcgttggttacgtacgtgattctggatccgtttcccagtacgttttaagcgaaatcaagccgctgcacaacttcctgactcaactgcagccgtttctggaactgaaacagaaacaggcaaacctggttctgaaaattatcgaacagctgccgtctgcaaaagaatccccggacaaattcctggaagtttgtacctgggtggatcagattgcagctctgaacgattctaagacgcgtaaaaccacttctgaaaccgttcgtgctgtgctggacagcctgagcgggaagaagaaatcctccccggcggccggtggatctgataagtataatcaggctctgtctaaatacaaccaagcactgtccaagtacaatcaggccctgtctggtggaggcggttccaacaaaaagttcctgctgtatcttgctggatttgtggattctgatggctccatcattgctcagataaaaccacgtcaatctaacaagttcaaacaccagctctccttgacttttgcagtcactcagaagacacaaagaaggtggttcttggacaaattggttgataggattggtgtgggctatgtctatgacagtggctctgtgtcagactaccgcctgtctgaaattaagcctcttcataactttctcacccaactgcaacccttcttgaagctcaaacagaagcaagcaaatctggttttgaaaatcatcgagcaactgccatctgccaaggagtcccctgacaagtttcttgaagtgtgtacttgggtggatcagattgctgccttgaatgactccaagaccagaaaaaccacctctgagactgtgagggcagttctggatagcctctctgagaagaaaaagtcctctccttag

SEQ ID NO: 113 - protein, 354 aa, Artificial Sequence
  Description of Artificial Sequence Synthetic polypeptide
MANTKYNEEFLLYLAGFVDGDGSIIAQINPNQSSKFKHRLRLTFYVTQKTQRRWFLDKLVDEIGVGYVRDSGSVSQYVLSEIKPLHNFLTQLQPFLELKQKQANLVLKIIEQLPSAKESPDKFLEVCTWVDQIAALNDSKTRKTTSETVRAVLDSLSGKKKSSPAAGGSDKYNQALSKYNQALSKYNQALSGGGGSNKKFLLYLAGFVDSDGSIIAQIKPRQSNKFKHQLSLTFAVTQKTQRRWFLDKLVDRIGVGYVYDSGSVSDYRLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 114 - DNA, 20 bp, Artificial Sequence
  Description of Artificial Sequence Synthetic primer
aggatctcctgtcatctcac

SEQ ID NO: 115 - DNA, 24 bp, Artificial Sequence
  Description of Artificial Sequence Synthetic primer
ctttcacagtcctgtacatcttgt

SEQ ID NO: 116 - protein, 9 aa, Artificial Sequence
  Description of Artificial Sequence Synthetic peptide
LAGLIDADG

SEQ ID NO: 117 - protein, 5 aa, Artificial Sequence
  Description of Artificial Sequence Synthetic peptide
LAGLI

SEQ ID NO: 118 - protein, 4 aa, Artificial Sequence
  Description of Artificial Sequence Synthetic peptide
DADG

SEQ ID NO: 119 - protein, 15 aa, Artificial Sequence
  Description of Artificial Sequence Synthetic peptide
SGISAYLLGLIIGDG

SEQ ID NO: 120 - protein, 14 aa, Artificial Sequence
  Description of Artificial Sequence Synthetic peptide
EQIAFIKGLYVAEG

SEQ ID NO: 121 - protein, 16 aa, Artificial Sequence
  Description of Artificial Sequence Synthetic peptide
NKEFLLYLAGFVDGDG

SEQ ID NO: 122 - protein, 25 aa, Artificial Sequence
  Description of Artificial Sequence Synthetic peptide
YYFANMLERIRLFNMREQLAFLAGF

SEQ ID NO: 123 - protein, 8 aa, Artificial Sequence
  Description of Artificial Sequence Synthetic peptide
ERIRLFNM

SEQ ID NO: 124 - protein, 20 aa, Artificial Sequence
  Description of Artificial Sequence Synthetic peptide
TQLQAMLERIRLFNMREFLL

SEQ ID NO: 125 - protein, 20 aa, Artificial Sequence
  Description of Artificial Sequence Synthetic peptide
TQLQAFLERIRLFNMREFLL

SEQ ID NO: 126 - protein, 20 aa, Artificial Sequence
  Description of Artificial Sequence Synthetic peptide
TQLQAMLERIRLYNMREFLL

SEQ ID NO: 127 - protein, 20 aa, Artificial Sequence
  Description of Artificial Sequence Synthetic peptide
TQLQAFLERIRLYNMREFLL

SEQ ID NO: 128 - DNA, 35 bp, Artificial Sequence
  Description of Artificial Sequence Synthetic primer
gtacagcaggaactcttcgttatatttggtattgg

SEQ ID NO: 129 - protein, 6 aa, Artificial Sequence
  Description of Artificial Sequence Synthetic 6xHis tag
HHHHHH